(19) United States
(12) Patent Application Publication
(10) Pub. No.: US 2003/0188344 A1
(43) Pub. Date: Oct. 2, 2003

(54) Compositions and methods for agrobacterium transformation of plants

(76) Inventors: David G. Lynn, Atlanta, GA (US); Jin Zhang, San Diego, CA (US); Angela Campbell, Hatboro, PA (US); Andrew Binns, Wallingford, PA (US)

Correspondence Address: Steven L. Highlander, FULBRIGHT & JAWORSKI L.L.P., 600 Congress Avenue, Suite 2400, Austin, TX, 78701, US

(21) Appl. No.: 09/735,701

(22) Filed: Dec. 12, 2000

(51) Int. Cl. C12N 15/84; A01H 5/00; C12N 5/04; C12N 15/82

(52) U.S. Cl. 800/294; 435/320.1; 435/252.2; 435/469

(57) ABSTRACT

The present invention is directed to variants of Agrobacterium tumefaciens. These variants are either resistant to the effects of MDIBOA/DIMBOA, or hypersensitive to phenolic induction. These variants are improved over wild-type Agrobacterium in their ability to transform plant cells. Also provided are methods for their selection. In a distinct embodiment, there also is provided a modified Ti plasmid that increases the ability of an Agrobacterium strain to transform host cells. The plasmid contains virA and virG genes, under the control of the coliphage T5 P_{N25} promoter.

The United States government may own rights in this application by virtue of funding through the National Institutes of Health and Grant No. GM47369.

### BACKGROUND OF THE INVENTION

1. Field of the Invention

The present invention relates generally to molecular and cellular biology. More specifically, it relates to methods and compositions for improving transformation in plants using Agrobacterium tumefaciens.

2. Description of the Related Art

Agrobacterium tumefaciens represents the only known organism that routinely mediates inter-Kingdom gene transfer, and, as such, has been exploited as a natural vector for the incorporation of foreign genes into higher plants. Zupan & Zambriski, 1995; Hooykaas and Beijerbergen, 1994; Christie, 1997; Birch, 1997. A key first step in Agrobacterium infection involves the virulence (vir) pathway. When virulent A. tumefaciens infects a wound site, the vir genes on the resident tumor-inducing (Ti) plasmid are expressed. Their activities result in the production of both the DNA intermediate that will be transferred, and the membrane-bound DNA transfer machinery. However, it is well known that most cereal crops are resistant to transformation, and studies have identified a limitation in signal-induced gene expression, most prominently in seedling root tissue. Chilton, 1993; Raineri et al., 1993; Hansen et al., 1994; Heth et al., 1997; Shen et al., 1993.

The expression of the vir regulon is controlled by virA and virG, two genes homologous to the ‘two-component’ regulatory systems utilized in many bacteria to respond appropriately to environmental signals. Winans, 1992; Pirrung, 1999; McEvoy & Dahlquist, 1997; Heath et al., 1995; Swanson et al., 1994. In this case, VirA serves as the membrane-localized histidine autokinase transmitter (sometimes referred to as ‘sensor kinase’) and VirG as the response regulator. When the appropriate host recognition factors (xenognosins) are produced and accumulate at the wound site, VirG becomes phosphorylated and the expression of all the vir genes, including virA and virG, are upregulated.

The mechanism(s) that couple transmitter kinase activity to input (signal) control in two component systems remain largely unknown, and physical data on signal recognition is scant when compared to the vast numbers of systems so far described. Parkinson & Kofoid, 1992; Parkinson, 1993. Within the known two-component systems, signal recognition by the transmitter kinase can be either direct or indirect, and the effect of signs recognition at a structural and mechanistic level is not generally understood. In the case of VirA/VirG, signal input through VirA is quite complex. Several different classes of xengnostic signals produced at the wound site, including pH, sugars, and phenolics, are recognized and synergistically control vir gene expression. The low pH requirement can be altered by mutations within VirA, but the actual recognition site has not been defined. Melchers et al., 1989; Turk et al., 1991. Monosaccharides, including glucose and arabinose, interact with a chromosomally encoded sugar binding protein, ChvE, and this complex affects the sensitivity, specificity and maximal response of the system to certain phenols. Cangelosi et al., 1990; Shimoda et al., 1990; Banta et al., 1994; Turk et al., 1993; Machida et al., 1993. Point mutations or deletions in the periplasmic domain of VirA, both in the putative ChvE binding site and distal to it, can abolish the ability of VirA to respond to sugars.

Xenognostic phenol recognition is the critical step in VirA/VirG activation. Whereas the phenols can induce vir gene expression in the absence of inducing sugars, the sugars alone are not sufficient. Cangelosi et al., 1990; Shimoda et al., 1990; Banta et al., 1994; Turk et al., 1993; Machida et al., 1993. Although VirA is the critical transmitter kinase of phenol-mediated vir gene activation, the nature of the perceiving element has not been conclusively defined. Molecular genetic studies of VirA indicate that the linker/kinase domain is the smallest fragment that can function in phenol-mediated vir gene expression. Chang & Winans, 1992. Consistent with a role in phenol perception, point mutation within and deletion of the linker domain eliminates the response to phenols. Doty et al., 1996; Turk et al., 1994; Chang et al., 1996. While genetic studies appeared to indicate that the specificity of the phenols involved in induction is defined by the resident Ti plasmid, more recent work indicates that the spectrum of phenols recognized by the system is determined by the relative abundance of both ChvE and the inducing sugars. Lee et al., 1995; Peng et al., 1998. Finally, deletion of, or point mutations in, the receiver domain of VirA can also increase the range of phenols that are able to induce vir gene expression. Chang et al., 1996.

Beyond the fact that the genetic basis of the phenol specificity in the Agrobacterium vir inducing system is not clear, evidence for a specific physical interaction between VirA and the xenognostic phenol has not been presented. In fact, affinity labeling and affinity chromatography approaches to identify specific phenol binding proteins in Agrobacterium have, instead, identified a few small proteins that are not encoded on the Ti plasmid. Lee et al., 1992; Dye & Delmotte, 1997. Genetic data indicating the involvement of these proteins in vir gene expression has, however, not been obtained. These seemingly conflicting data, and the fact that more than 80 different phenols are perceived by Agrobacterium and induce vir gene expression, raises further questions as to whether there could be a specific receptor. For example, there could be a more general physical manifestation of phenol exposure that controls the response. As such, a greater understanding of the phenolic dependency of Agrobacterium, along identification of inhibitory factors of signal induction, are required to increase its potential as a gene transfer vehicle.

### SUMMARY OF THE INVENTION

Thus, according to the present invention, there is provided a method for selecting a MDIBOA/DIMBOA resistant Agrobacterium strain comprising (a) providing an Agrobacterium cell; (b) culturing said Agrobacterium cell with a phenolic inducer of the vir pathway and under other conditions supporting Agrobacterium replication, but including MDIBOA/DIMBOA at concentrations sufficient to inhibit Agrobacterium replication; and (c) isolating an Agrobacterium cell that has replicated in the culture of step (b), thereby selecting MDIBOA/DIMBOA a resistant Agrobacterium strain.

The Agrobacterium cell of may further comprises an antibiotic resistance gene under the control of a promoter that is upregulated by a vir gene product, and the culture conditions may include the antibiotic, resistance to which is provided by the antibiotic resistance gene, at concentrations sufficient to inhibit Agrobacterium replication. The antibiotic resistance gene may kan^{r}, and the antibiotic may be kanamycin, for example, at about 40-50 μM.

The method may employ DIMBOA at about 50-100 μM. The phenolic induction may use acetosyringone, for example, at about 30-100 μM. The method may further comprise culturing in the presence of a sugar, for example, glucose, at an exemplary concentration of The method may further comprising making a replicate culture of the selected Agrobacterium cell; and culturing the selected Agrobacterium cell in the presence of MDIBOA/DIMBOA and antibiotic, but without said phenolic inducer, wherein a cell that replicates is identified as having a constitutively activated vir pathway, and a cell that does not replicate is identified as having a vir-mediated MDIBOA/DIMBO resistant mutation.

The method may further comprise treating a replicate culture of the Agrobacterium cell of step (a) under conditions supporting Agrobacterium replication, but excluding said phenolic inducer and including MDIBOA/DIMBOA at concentrations sufficient to inhibit Agrobacterium replication.

In another embodiment, there is provided a method for selecting a phenol hypersensitive Agrobacterium strain comprising (a) providing an Agrobacterium cell that comprises an antibiotic resistance gene under the control of a promoter that is upregulated by a vir gene product; (b) culturing said Agrobacterium cell with varying levels of a phenolic inducer and under other conditions supporting Agrobacterium replication, but including antibiotic resistance to which is provided by the antibiotic resistance gene, at concentrations sufficient to inhibit Agrobacterium replication; and (c) isolating an Agrobacterium cell receiving the lowest level of phenolic induction that has replicated in the culture of step (b), thereby selecting a first phenol hypersensitive Agrobacterium strain. The method may further comprise repeating the steps (a)-(c) with said first phenol hypersensitive Agrobacterium cell, thereby obtaining a second phenol hypersensitive Agrobacterium cell.

The antibiotic resistance gene may be kan^{r}, and said antibiotic may be kanamycin, for example, at about 40-50 μM. The phenolic induction may use acetosyringone, for example, at 20 about 10 μM. The method may further comprise culturing in the presence of a sugar, for example, arabinose, at an exemplary concentration of0.1-1%.

In yet another embodiment, there is provided a MDIBOA/DIMBOA resistant Agrobacterium strain selected according to the method comprising:

(a) providing an Agrobacterium cell;

(b) culturing said Agrobacterium cell with a phenolic inducer and under other conditions supporting Agrobacterium replication, but including

MDIBOA/DIMBOA at concentrations sufficient to inhibit Agrobacterium replication; and

(c) isolating an Agrobacterium cell that has replicated in the culture of step (b),

thereby selecting MDIBOA/DIMBOA a resistant Agrobacterium cell.

In still another embodiment, there is provided a phenol hypersensitive Agrobacterium strain comprising selected according to the method:

(a) providing an Agrobacterium cell that comprises an antibiotic resistance gene under the control of a promoter that is upregulated by a Vir gene product;

(b) culturing said Agrobacterium cell with varying levels of a phenolic inducer and under other conditions supporting Agrobacterium replication, but including antibiotic resistance to which is provided by the antibiotic resistance gene, at concentrations sufficient to inhibit Agrobacterium replication; and

(c) isolating an Agrobacterium cell receiving the lowest level of phenolic induction that has replicated in the culture of step (b),

thereby selecting a first phenol hypersensitive Agrobacterium cell.

In still yet another embodiment, there is provided a method for producing a MDIBOA/DIMBOA resistant Agrobacterium comprising (a) providing an Agrobacterium cell; (b) culturing said Agrobacterium cell with a phenolic inducer and under other conditions supporting Agrobacterium replication, but including MDIBOA/DIMBOA at concentrations sufficient to inhibit Agrobacterium replication; (c) isolating an Agrobacterium cell that has replicated in the culture of step (b); (d) producing a culture of the isolated Agrobacterium.

Similarly, the patent provides a method for producing a phenol hypersensitive Agrobacterium strain comprising (a) providing an Agrobacterium cell that comprises an antibiotic resistance gene under the control of a promoter that is upregulated by a vir gene product; (b) culturing said Agrobacterium cell with varying levels of a phenolic inducer and under other conditions supporting Agrobacterium replication, but including antibiotic resistance to which is provided by the antibiotic resistance gene, at concentrations sufficient to inhibit Agrobacterium replication; (c) isolating an Agrobacterium cell receiving the lowest level of phenolic induction that has replicated in the culture of step (b); and (d) producing a culture of the isolated Agrobacterium.

Also provided a DNA libraries prepared from any of the phenol hypersensitive or MDIBOA/DIMBOA resistant strains.

Further embodiments include a method for transducing a plant using a MDIBOA/DIMBOA resistant Agrobacterium strain comprising (a) providing a plant cell; (b) contacting said plant cell with said MDIBOA/DIMBOA resistant Agrobacterium; and (c) culturing said plant cell under conditions suitable for Agrobacterium-mediated transformation. The plant cell may be a monocot or a dicot. The Agrobacterium may harbor a heterologous gene, for example, a gene that affects one or more performance traits in a plant of said plant cell. The method may further comprise obtaining seed or progeny of said plant.

Similarly, the present invention provides for a method for transducing a plant using a phenol hypersensitive Agrobacterium strain comprising (a) providing a plant cell; (b) contacting said plant cell with said phenol hypersensitive Agrobacterium; and (c) culturing said plant cell under conditions suitable for Agrobacterium-mediated transformation. The plant cell may be a monocot or a dicot. The Agrobacterium may harbor a heterologous gene, for example, a gene that affects one or more performance traits in a plant of said plant cell. The method may further comprise obtaining seed or progeny of said plant.

Also provided is a plasmid comprising the P_{N25} promoter of coliphage T5 and the virA and virG genes of Agrobacterium tumefaciens, wherein said virA and virG genes are under the transcriptional control of said promoter. The plasmid backbone may be derived from a broad host range vector, for example, pJB20. The virA gene may encode a functional deletion mutant of virA, for example, a mutant lacking the first 284 amino acid residues of VirA. Also provided are host cells comprising this plasmid.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—HPLC trace of maize root exudate.

FIG. 2—Inhibition of vir expression by 1. Effect of 1 on virB::lacZ gene expression induced by 1 mM AS (—o—), 100 μM AS (—II—) or with no AS (—⋄—) in A348-3 carrying pAB 123-22 and pSW209Ω with comparison to its inhibitory effect in A348-3/pMutA G665D, pSW209Ω (— —).

FIG. 3—Induction of vir expression. β-galactosidase induction by AS in wild-type A348 (—0—), JZ103 (—⋄—), and JZ104 (—II—) carrying pSW209 (virB::lacZ). Mean±standard deviation (n=3).

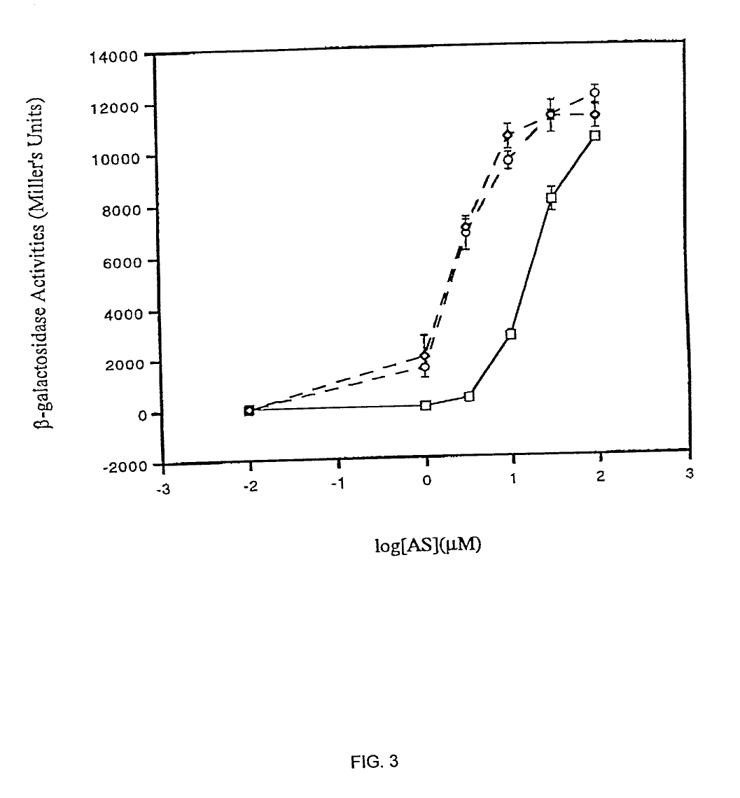

FIG. 4—Inhibition of vir expression by DIMBOA. Effect of DIMBOA on virB::lacZ gene expression induced by 10 μM AS in the presence of the 100 mM glucose in wild-type A348 (—II—), JZ103 (—0—), and JZ104 (—⋄—) carrying pSW209 (virB::lacZ). Mean±standard deviation (n=3).

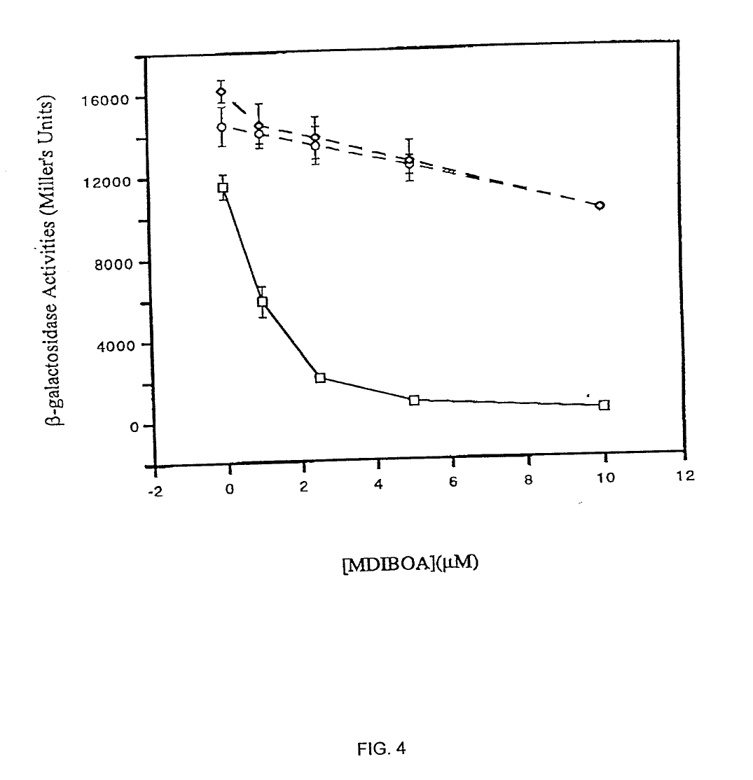

FIG. 5—Inhibition of vir expression by 1. Effect of 1 on virB::lacZ gene expression induced by 10 μM AS in the presence of the 100 mM glucose in wild-type A348 (—II—), JZ103 (—0—), and JZ104 (—⋄—) carrying pSW209 (virB::lacZ). Mean±standard deviation (n=3).

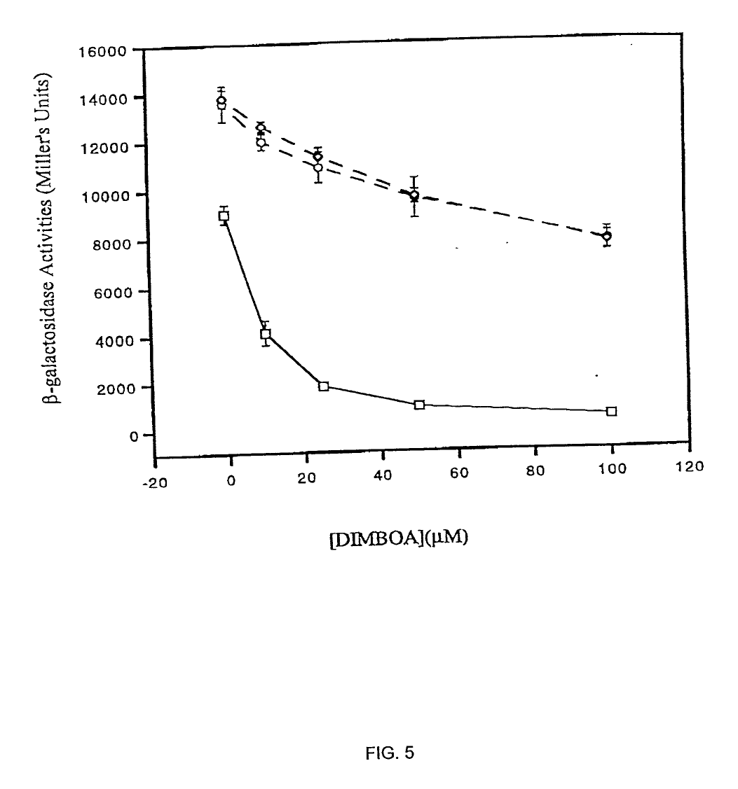

FIG. 6—Structures for 2hdroxy-4,7-dimethoxybenzoxazin-3-one [1], DIMBOA and acetosyringone (AS).

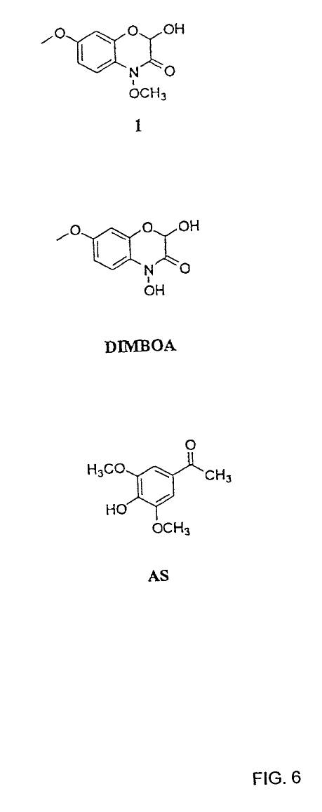

FIG. 7—Depictions of chemical discussed in Example 2.

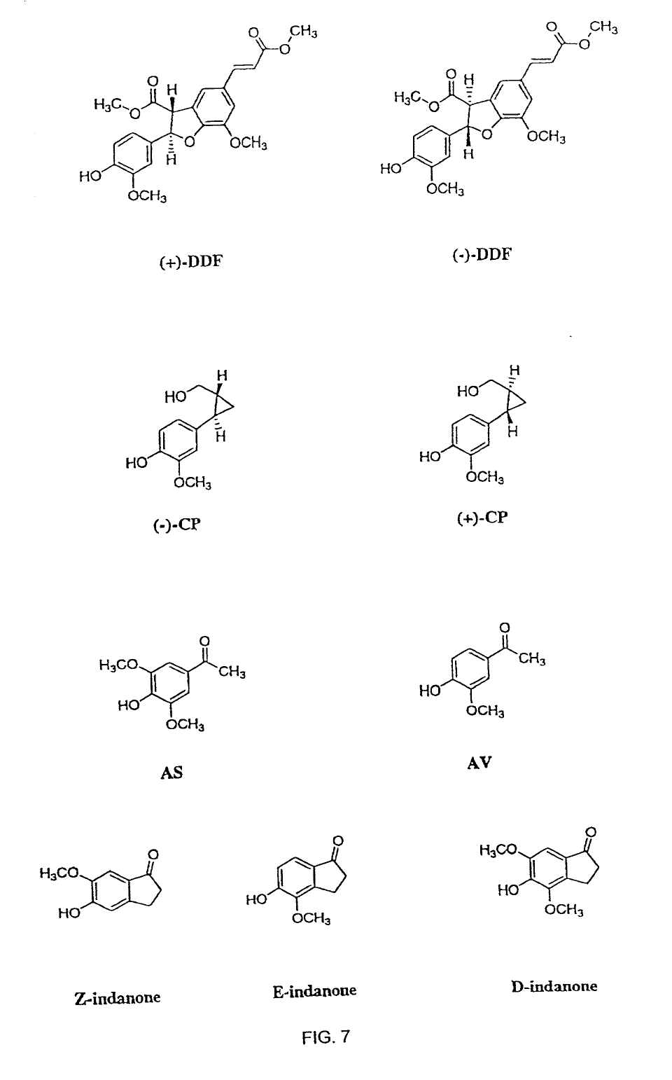

FIGS. 8A and 8B—Induction of vir expression. β-Galactosidase expression in A348 carrying pSW209 (virB::lacZ) induced with FIG. 8A: (−)-trans-DDF(—II—), (+)-trans-DDF(—⋄—); and FIG. 8B: (+)-transCP (— —) and (-trans-CP (—O—). Expressed as mean±standard deviation (n=3).

FIG. 9—Cell Growth. Kanamycin resistant growth of wild-type A348 and AB140, each carrying pAC2 (virB^{P}/nptII), expressed as a percentage of control with AS induction in wild-type A348/pAC2(—II—), AV in wild-type A348/pAC2(— —), AS in AB 140/pAC2,(—⋄—), and AV in AB1 40/pAC2(—⋄—). Expressed as mean±standard deviation (n=3).

FIG. 10—Tumor induction. Tobacco leaf square pieces were co-cultivated with wild-type A348 (—II—) and AB 140 (—⋄—) in the presence of various concentrations of AS. After 12 days, the leaf pieces were scored for the mean number of tumors per leaf square±standard error (n=20).

FIGS. 11A, 11B and 11C—Induction of vir expression. β-Galactosidase induction with dimethoxy-indanone (FIG. 11A), Z-indanone (FIG. 11B), and E-indanone (FIG. 11C) in A348 (—II—), AB140 (—⋄—), and AB144 (—O—) carrying pSW209 (virB^{P}/lacZ). Mean±standard deviation (n=3).

FIGS. 12A, 12B, 12C and 12D—Induction of vir expression. β-Galactosidase induction in strains grown at varying AS concentrations in ABIM plus 1% glycerol in the presence (FIG. 12A and FIG. 12C) and absence (FIG. 12B and FIG. 12D) of the 0.1% arabinose. FIG. 12A and FIG. 12B: A348/pSW209 (—II—) and AB 140/pSW209 (—⋄—), FIG. 12C and FIG. 12D: A348/pSW209 (—II—) and AB 147/pSW209 (—O—). Mean±standard deviation (n=3).

FIG. 13—Induction of vir expression. Ti plasmids of wild-type A348 and AB 140 were conjugated into UIA143/pSW209 and β-galactosidase expression was measured in UIA143/pTiA^{wt} (—II—) and UIA143/pTiA6¹⁴⁰ (—⋄—). Mean±standard deviation (n=3).

FIG. 14—Model of D-indanone receptor. Regions A, B and C represent domains of the Xbp involved in binding.

FIGS. 15A and 15B—vir gene expression. (FIG. 15A) A. tumefaciens strain A348-3/pSW209Ω carrying (▴) pMutA(G665D); (●) pYW39 (P_{N25}-6×His-virA(Δ1 284, G665D); (¤) pYW40 (P_{N25}-virA(Δ1-284, G665D)); and (◯) pYW15a (control were grown at 20° C. in IM containing 1% glycerol and various AS. β-galactosidase activity was determined after 20 hrs and the results are expressed as percentage of the maximal β-galactosidase for each strain at 300 μM AS. (FIG. 15B) Time course for expression in (¤) A348/pSW209 (P_{virG}-virG;) and A136/pSW209 carrying (●) pYW48 (P_{N25}-6×is-virG) or (◯) pYW15b (negative control). Strains were induced in IM, pH 5.5, with 1% glucose and 100 μM AS. Aliquots were removed at indicated time intervals and assayed for β-galactosidase activity.

### DETAILED DESCRIPTION OF THE INVENTION

Agrobacterium mediated transformation is a powerful technique for the creation of transgenic cells and plants. Unfortunately, many plants have defense mechanisms that make them resistant to Agrobacterium transformation. The present inventors have identified MDIBOA/DIMBOA as the major constituent of maize seedling exudate, and show it to be the most potent and specific inhibitor of the initial step in maize transformation known. The present invention seeks to exploit this observation by creating compositions and methods that circumvent the inhibitory activity of DIMBOA.

The present invention also seeks to approach the problem of plant resistance to Agrobacterium from another angle. It is known that the virulence (vir) genes of Agrobacterium are required for gene expression and replication. Various different mechanisms are known by which vir genes are controlled, including signaling through sugars, pH and phenolics. Phenolics are particularly important, as they can induce vir gene expression alone. By increasing the sensitivity of Agrobacterium to these compounds, the present inventors have provided yet another means to host resistance to Agrobacterium transformation.

Finally, the present inventors have developed novel expression constructs for use in plant and bacterial transformation. These constructs employs various elements of the Agrobacterium Ti plasmid, and also include the PN25 promoter of coliphage T5. By placing certain key vir genes under the control of this promoter, it is possible to simplify the complex regulation of this pathway, and increase expression in even hostile host cell environments.

I. Plant Transformation Constructs

Transformation constructs designed for the expression of selected genes in plants form an important part of the invention. The construction of such vectors will be known to those of skill of the art in light of the present disclosure (see for example, Sambrook et al., 1989; Gelvin et al., 1990). The choice of the particular selected genes will depend on the purpose of the transformation. One of the major purposes of transformation of crop plants is to add commercially desirable, agronomically important traits to the plant. Such traits include, but are not limited to, herbicide resistance or tolerance; insect resistance or tolerance; disease resistance or tolerance (viral, bacterial, fungal, nematode); stress tolerance and/or resistance, as exemplified by resistance or tolerance to drought, heat, chilling, freezing, excessive moisture, salt stress, or oxidative stress; increased yields; food content and makeup; physical appearance; male sterility; drydown; standability; prolificacy; starch properties; oil quantity and quality, and the like.

In certain embodiments of the invention, the present inventors contemplate the transformation of a recipient cell with more than one transformation construct. Two or more transgenes can be created in a single transformation event using either distinct selected-gene encoding vectors, or using a single vector incorporating two or more gene coding sequences. Using this method, the assumption is made that a certain percentage of cells in which a marker has been introduced also have received the other gene(s) of interest. Thus, not all cells selected by means of the marker, will express the other genes of interest which had been presented to the cells concurrently. Of course, any two or more transgenes of any description, such as those conferring, for example, herbicide, insect, disease (viral, bacterial, fungal, nematode) or drought resistance, male sterility, drydown, standability, prolificacy, starch properties, oil quantity and quality, or those increasing yield or nutritional quality may be employed as desired.

In other embodiments of the invention, it is contemplated that one may wish to employ replication-competent viral vectors for plant transformation. Such vectors include, for example, wheat dwarf virus (WDV) “shuttle” vectors, such as pW1-11 and PW1-GUS (Ugaki et al., 1991). These vectors are capable of autonomous replication in maize cells as well as E. coli, and as such may provide increased sensitivity for detecting DNA delivered to transgenic cells. A replicating vector also may be useful for delivery of genes flanked by DNA sequences from transposable elements such as Ac, Ds, or Mu. It has been proposed that transposition of these elements within the maize genome requires DNA replication (Laufs et al., 1990). It also is contemplated that transposable elements would be useful for introducing DNA fragments lacking elements necessary for selection and maintenance of the plasmid vector in bacteria, e.g., antibiotic resistance genes and origins of DNA replication. It also is proposed that use of a transposable element such as Ac, Ds, or Mu would actively promote integration of the desired DNA and hence increase the frequency of stably transformed cells.

Vectors used for plant transformation may include, for example, plasmids, cosmids, YACs (yeast artificial chromosomes), BACs (bacterial artificial chromosomes) or any other suitable cloning system. The utilization of cloning systems with large insert capacities will allow introduction of large DNA sequences comprising more than one selected gene. Introduction of such sequences may be facilitated by use of bacterial or yeast artificial chromosomes (BACs or YACs, respectively), or even plant artificial chromosomes. For example, the use of BACs for Agrobacterium-mediated transformation was disclosed by Hamilton et al. (1996).

Particularly useful for transformation are expression cassettes which have been isolated from such vectors. DNA segments used for transforming plant cells will, of course, generally comprise the cDNA, gene or genes which one desires to introduced into and have expressed in the host cells. These DNA segments can further include structures such as promoters, enhancers, polylinkers, or even regulatory genes as desired. The DNA segment or gene chosen for cellular introduction will often encode a protein which will be expressed in the resultant recombinant cells resulting in a screenable or selectable trait and/or which will impart an improved phenotype to the resulting transgenic plant. However, this may not always be the case, and the present invention also encompasses transgenic plants incorporating non-expressed transgenes. Preferred components likely to be included with vectors used in the current invention are as follows.

A. Regulatory Elements

Constructs prepared in accordance with the invention may comprise a wide variety of regulatory elements. In one embodiment of the invention, a promoter operably linked to a selected gene will be included on a transformation construct. By the term “operably linked,” when used in relation to a promoter and a selected gene, it is meant that the promoter is capable of directing the expression of the selected gene in a host plant cell transformed with the selected gene operably linked to the promoter. Where the term “operably linked” is used in relation to a heterologous gene, it is meant that the element is positioned in relative proximity to the heterologous gene such that it is capable of regulating expression of the heterologous gene.

The selection of a promoter for use with a selected gene is made based upon the promoter's ability to direct the transformed plant cell's or transgenic plant's transcriptional activity to the coding region. Additionally, it is believed that some promoters may function more efficiently when used in conjunction with other elements. Such selections may be made using the assays and procedures as described below.

Useful plant promoters include those that are inducible, viral, synthetic, constitutive as described (Poszkowski et al., 1989; Odell et al., 1985), temporally regulated, spatially regulated, and spatio-temporally regulated (Chau et al., 1989). Exemplary constitutive promoters include the CaMV 35S promoter (Odell et al., 1985), histone, CaMV 19S (Lawton et al., 1987), nos (Ebert et al., 1987), Adh (Walker et al., 1987), sucrose synthase (Yang & Russell, 1990), α-tubulin, actin (Wang et al., 1992), cab (Sullivan et al., 1989), PEPCase (Hudspeth & Grula, 1989) and R gene complex-associated promoters (Chandler et al., 1989). Where the promoter is a near-constitutive promoter, increases in polypeptide expression generally are found in a variety of transformed plant tissues (e.g., callus, leaf, seed and root).

Exemplary tissue-specific promoters include lectin (Vodkin et al., 1983; Lindstrom et al., 1990), corn alcohol dehydrogenase 1 (Vogel et al., 1989; Dennis et al., 1984); corn light harvesting complex (Simpson, 1986; Bansal et al., 1992), corn heat shock protein (Odell et al., 1985; Rochester et al., 1986), pea small subunit RuBP carboxylase (Poulsen et al., 1986; Cashmore et al., 1983), Ti plasmid mannopine synthase (Langridge et al., 1989), Ti plasmid nopaline synthase (Langridge et al., 1989), petunia chalcone isomerase (Van Tunen et al., 1988), bean glycine rich protein 1 (Keller et al., 1989), truncated CaMV 35s (Odell et al., 1985; Benfey et al., 1989), potato patatin promoters (Wenzler et al., 1989), root cell promoters (Conkling et al., 1990), tissue specific enhancers (Fromm et al., 1989), maize zein (Reina et al., 1990; Kriz et al., 1987; Wandelt and Feix, 1989; Langridge and Feix, 1983; Reina et al., 1990), globulin-1 (Belanger and Kriz et al., 1991), and chalcone synthase promoters (Franken et al., 1991).

Examples of inducible promoters include ABA- and turgor-inducible promoters and the promoter of the auxin-binding protein gene (Scwob et al., 1993; Genbank Accession No. L08425). Still other potentially useful promoters include the UDP glucose flavonoid glycosyl-transferase gene promoter (Ralston et al., 1988); MPI proteinase inhibitor (Cordero et al., 1994), and the glyceraldehyde-3-phosphate dehydrogenase gene promoter (Kohler et al., 1995; Quigley et al., 1989; Martinez et al., 1989), as well as promoters of chloroplast genes (Genbank Accession No. X86563).

In addition to promoters, other types of elements can regulate gene expression. One such element is the DNA sequence between the transcription initiation site and the start of the coding sequence, termed the untranslated leader sequence. The leader sequence can influence gene expression and compilations of leader sequences have been made to predict optimum or sub-optimum sequences and generate “consensus” and preferred leader sequences (Joshi, 1987). Preferred leader sequences include those which have sequences predicted to direct optimum expression of the attached gene, i.e., to include a preferred consensus leader sequence which may increase or maintain mRNA stability and prevent inappropriate initiation of translation. Sequences that are derived from genes that are highly expressed in plants, and in maize in particular, will be most preferred.

Transcription enhancers or duplications of enhancers can be used to increase expression. These enhancers often are found 5′ to the start of transcription in a promoter that functions in eukaryotic cells, but can also often be inserted in the forward or reverse orientation 5′ or 3′ to the coding sequence. Examples of enhancers include elements from the CaMV 35S promoter, octopine synthase genes (Ellis et al., 1987; Benfey et al., 1989), the rice actin 1 gene, the maize alcohol dehydrogenase gene, the maize shrunken 1 gene and promoters from non-plant eukaryotes (e.g., yeast; Ma et al., 1988).

Specifically contemplated for use in accordance with the present invention are vectors which include the ocs enhancer element. This element was first identified as a 16 bp palindromic enhancer from the octopine synthase (ocs) gene of Agrobacterium (Ellis et al., 1987), and is present in at least 10 other promoters (Bouchez et al., 1989). It is proposed that the use of an enhancer element, such as the ocs element and particularly multiple copies of the element, may be used to increase the level of transcription from adjacent promoters when applied in the context of monocot transformation.

Ultimately, the most desirable DNA segments for introduction into a plant genome may be homologous genes or gene families which encode a desired trait, and which are introduced under the control of novel promoters, enhancers, or terminators. Tissue-specific regulatory regions may be particularly useful. Indeed, it is envisioned that a particular use of the present invention may be the production of transformants comprising a transgene which is expressed in a tissue-specific manner. For example, insect resistance genes may be expressed specifically in the whorl and collar/sheath tissues which are targets for the first and second broods, respectively, of European Corn Borer (ECB). Likewise, genes encoding proteins with particular activity against rootworm may be targeted directly to root tissues. In addition, expression of certain genes which affect the nutritional composition of the grain must be targeted to the seed, e.g., endosperm or embryo.

Vectors for use in tissue-specific targeting of gene expression in transgenic plants typically will include tissue-specific promoters and also may include other tissue-specific control elements such as enhancer sequences. Promoters which direct specific or enhanced expression in certain plant tissues in accordance with the invention will be known to those of skill in the art in light of the present disclosure.

It also is contemplated that tissue specific expression may be functionally accomplished by introducing a constitutively expressed gene (all tissues) in combination with an antisense gene that is expressed only in those tissues where the gene product is not desired. For example, a gene coding for the crystal toxin protein from B. thuringiensis (Bt) may be introduced such that it is expressed in all tissues using a constitutive promoter. Therefore, expression of an antisense transcript of the Bt gene in a maize kernel, using for example a zein promoter, would prevent accumulation of the Bt protein in seed. Hence the protein encoded by the introduced gene would be present in all tissues except the kernel. Furthermore, it is contemplated that promoters combining elements from more than one promoter may be useful. For example, U.S. Pat. No. 5,491,288 discloses combining a Cauliflower Mosaic Virus promoter with a histone promoter.

Alternatively, one may wish to obtain novel, tissue-specific promoter sequences for use according to the present invention. To achieve this, one may first isolate cDNA clones from the tissue concerned and identify those clones which are expressed specifically in that tissue, for example, using Northern blotting. Ideally, one would like to identify a gene that is not present in a high copy number, but which gene product is relatively abundant in specific tissues. The promoter and control elements of corresponding genomic clones may then be localized using the techniques of molecular biology known to those of skill in the art.

Another useful method for identifying tissue-specific promoters is differential display (see, e.g., U.S. Pat. No. 5,599,672, the disclosure of which is specifically incorporated herein by reference in its entirety). In differential display, mRNAs are compared from different tissue types. By identifying mRNA species which are present in only a particular tissue type, or set of tissues types, one can identify the corresponding genes which are expressed is a tissue specific manner. The RNAs can be transcribed by reverse transcriptase to produce a cDNA, and the cDNA in turn be used to isolate clones containing the full-length genes. As specifically disclosed herein, the cDNA also can be used to isolate homologous or homologous promoters, enhancers or terminators from the respective gene using, for example, suppression PCR.

It is contemplated that expression of some genes in transgenic plants will be desired only under specified conditions. For example, it is proposed that expression of certain genes that confer resistance to environmental stress factors such as drought will be desired only under actual stress conditions. It further is contemplated that expression of such genes throughout a plants development may have detrimental effects. It is known that a large number of genes exist that respond to the environment. For example, expression of some genes such are regulated by light as mediated through phytochrome. Other genes are induced by secondary stimuli. For example, synthesis of abscisic acid (ABA) is induced by certain environmental factors, including but not limited to water stress. A number of genes have been shown to be induced by ABA (Skriver and Mundy, 1990). It also is anticipated that expression of genes conferring resistance to insect predation would be desired only under conditions of actual insect infestation. Therefore, for some desired traits, inducible expression of genes in transgenic plants will be desired.

It is proposed that, in some embodiments of the present invention, expression of a gene in a transgenic plant will be desired only in a certain time period during the development of the plant. Developmental timing frequently is correlated with tissue specific gene expression. For example, expression of zein storage proteins is initiated in the endosperm about 10 days after pollination.

It also is contemplated that it may be useful to target DNA itself within a cell. For example, it may be useful to target introduced DNA to the nucleus as this may increase the frequency of transformation. Within the nucleus itself it would be useful to target a gene in order to achieve site specific integration. For example, it would be useful to have a gene introduced through transformation replace an existing gene in the cell.

B. Terminators

Transformation constructs prepared in accordance with the invention may include a terminator. The terminator is capable of acting as a signal to terminate transcription and allow for the poly-adenylation of the resultant mRNA. The terminator may be operably linked to a selected heterologous gene. In this instance, the terminator will typically be located at the 3′ end of the selected gene, thereby being capable of serving as a transcriptional terminator. However, it is contemplated by the inventors that the terminator could be placed at any other position relative to a selected gene including 5′ or within the selected gene. The transformation constructs prepared in accordance with the invention may additionally include as many selected genes as may physically be placed on the vector used. The additional genes may be linked to a terminator or alternatively, to other transcriptional terminators.

Preferred 3′ elements are those from the nopaline synthase gene of Agrobacterium tumefaciens (nos 3′ end) (Bevan et al., 1983), the terminator for the T7 transcript from the octopine synthase gene of Agrobacterium tumefaciens, and the 3′ end of the protease inhibitor I or II genes from potato or tomato. Alternatively, one also could use a gamma coixin, oleosin 3 or other terminator from the genus Coix.

C. Transit or Signal Peptides

Sequences that are joined to the coding sequence of an expressed gene, which are removed post-translationally from the initial translation product and which facilitate the transport of the protein into or through intracellular or extracellular membranes, are termed transit (usually into vacuoles, vesicles, plasmids and other intracellular organelles) and signal sequences (usually to the endoplasmic reticulum, golgi apparatus and outside of the cellular membrane). By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed.

A particular example of such a use concerns the direction of a protein conferring herbicide resistance, such as a mutant EPSPS protein, to a particular organelle such as the chloroplast rather than to the cytoplasm. This is exemplified by the use of the rbcS transit peptide, the chloroplast transit peptide described in U.S. Pat. No. 5,728,925, or the optimized transit peptide described in U.S. Pat. No. 5,510,471, which confers plastid-specific targeting of proteins. In addition, it may be desirable to target certain genes responsible for male sterility to the mitochondria, or to target certain genes for resistance to phytopathogenic organisms to the extracellular spaces, or to target proteins to the vacuole. A further use concerns the direction of enzymes involved in amino acid biosynthesis or oil synthesis to the plastid. Such enzymes include dihydrodipicolinic acid synthase which may contribute to increasing lysine content of a feed.

D. Marker Genes

By employing a selectable or screenable marker gene as, or in addition to, the expressible gene of interest, one can provide or enhance the ability to identify transformants. “Marker genes” are genes that impart a distinct phenotype to cells expressing the marker gene and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). Of course, many examples of suitable marker genes are known to the art and can be employed in the practice of the invention.

Included within the terms selectable or screenable marker genes also are genes which encode a “secretable marker” whose secretion can be detected as a means of identifying or selecting for transformed cells. Examples include markers which encode a secretable antigen that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extension or tobacco PR-S).

With regard to selectable secretable markers, the use of a gene that encodes a protein that becomes sequestered in the cell wall, and which protein includes a unique epitope is considered to be particularly advantageous. Such a secreted antigen marker would ideally employ an epitope sequence that would provide low background in plant tissue, a promoter-leader sequence that would impart efficient expression and targeting across the plasma membrane, and would produce protein that is bound in the cell wall and yet accessible to antibodies. A normally secreted wall protein modified to include a unique epitope would satisfy all such requirements.

One example of a protein suitable for modification in this manner is extensin, or hydroxyproline rich glycoprotein (HPRG). The use of maize HPRG (Steifel et al., 1990) is preferred, as this molecule is well characterized in terms of molecular biology, expression and protein structure. However, any one of a variety of extensins and/or glycine-rich wall proteins (Keller et al., 1989) could be modified by the addition of an antigenic site to create a screenable marker.

One exemplary embodiment of a secretable screenable marker concerns the use of a maize sequence encoding the wall protein HPRG, modified to include a 15 residue epitope from the pro-region of murine interleukin-1-β (IL-1-β). However, virtually any detectable epitope may be employed in such embodiments, as selected from the extremely wide variety of antigen:antibody combinations known to those of skill in the art. The unique extracellular epitope, whether derived from IL-1β or any other protein or epitopic substance, can then be straightforwardly detected using antibody labeling in conjunction with chromogenic or fluorescent adjuncts.

1. Selectable Markers

Potentially any selectable marker gene may be used, including but not limited to, a neo gene (Potrykus et al., 1985) which codes for kanamycin resistance and can be selected for using kanamycin, G418, paromomycin, etc.; a bar gene which confers bialaphos or phosphinothricin resistance; a mutant EPSP synthase protein (Hinchee et al., 1988) conferring glyphosate resistance; a nitrilase gene such as bxn from Klebsiella ozaenae which confers resistance to bromoxynil (Stalker et al., 1988); a mutant acetolactate synthase gene (ALS) which confers resistance to imidazolinone, sulfonylurea or other ALS inhibiting chemicals (European Patent Application 154,204, 1985); a methotrexate resistant DHFR gene (Thillet et al., 1988), a dalapon dehalogenase gene that confers resistance to the herbicide dalapon; or a mutated anthranilate synthase gene that confers resistance to 5-methyl tryptophan. Where a mutant EPSP synthase gene is employed, additional benefit may be realized through the incorporation of a suitable chloroplast transit peptide, CTP (U.S. Pat. No. 5,188,642) or OTP (U.S. Pat. No. 5,633,448) and use of a modified maize EPSPS gene (PCT Application WO 97/04103).

An illustrative embodiment of selectable marker genes capable of being used in systems to select transformants are the genes that encode the enzyme phosphinothricin acetyltransferase, such as the bar gene from Streptomyces hygroscopicus or the pat gene from Streptomyces viridochromogenes. The enzyme phosphinothricin acetyl transferase (PAT) inactivates the active ingredient in the herbicide bialaphos, phosphinothricin (PPT). PPT inhibits glutamine synthetase, causing rapid accumulation of ammonia and cell death (Murakami et al., 1986; Twell et al., 1989).

Where one desires to employ a bialaphos resistance gene in the practice of the invention, the inventor has discovered that particularly useful genes for this purpose are the bar or pat genes obtainable from species of Streptomyces (e.g., ATCC No. 21,705). The cloning of the bar gene has been described (Murakami et al., 1986; Thompson et al., 1987) as has the use of the bar gene in the context of plants (De Block et al., 1987; De Block et al., 1989; U.S. Pat. No. 5,550,318).

2. Screenable Markers

Screenable markers that may be employed include a β-glucuronidase (GUS) or uidA gene which encodes an enzyme for which various chromogenic substrates are known; an R-locus gene, which encodes a product that regulates the production of anthocyanin pigments (red color) in plant tissues (Dellaporta et al., 1988); a β-lactamase gene (Sutcliffe, 1978), which encodes an enzyme for which various chromogenic substrates are known (e.g., PADAC, a chromogenic cephalosporin); a xy/E gene (Zukowsky et al., 1983) which encodes a catechol dioxygenase that can convert chromogenic catechols; an α-amylase gene (Ikuta et al., 1990); a tyrosinase gene (Katz et al., 1983) which encodes an enzyme capable of oxidizing tyrosine to DOPA and dopaquinone which in turn condenses to form the easily-detectable compound melanin; a β-galactosidase gene, which encodes an enzyme for which there are chromogenic substrates; a luciferase (lux) gene (Ow et al., 1986), which allows for bioluminescence detection; an aequorin gene (Prasher et al., 1985) which may be employed in calcium-sensitive bioluminescence detection; or a gene encoding for green fluorescent protein (Sheen et al., 1995; Haseloff et al., 1997; Reichel et al., 1996; Tian et al., 1997; WO 97/41228).

Genes from the maize R gene complex are contemplated to be particularly useful as screenable markers. The R gene complex in maize encodes a protein that acts to regulate the production of anthocyanin pigments in most seed and plant tissue. Maize strains can have one, or as many as four, R alleles which combine to regulate pigmentation in a developmental and tissue specific manner. Thus, an R gene introduced into such cells will cause the expression of a red pigment and, if stably incorporated, can be visually scored as a red sector. If a maize line carries dominant alleles for genes encoding for the enzymatic intermediates in the anthocyanin biosynthetic pathway (C2, A1, A2, Bz1 and Bz2), but carries a recessive allele at the R locus, transformation of any cell from that line with R will result in red pigment formation. Exemplary lines include Wisconsin 22 which contains the rg-Stadler allele and TR112, a K55 derivative which is r-g, b, P1. Alternatively, any genotype of maize can be utilized if the C1 and R alleles are introduced together.

It further is proposed that R gene regulatory regions may be employed in chimeric constructs in order to provide mechanisms for controlling the expression of chimeric genes. More diversity of phenotypic expression is known at the R locus than at any other locus (Coe et al., 1988). It is contemplated that regulatory regions obtained from regions 5′ to the structural R gene would be valuable in directing the expression of genes for, e.g., insect resistance, herbicide tolerance or other protein coding regions. For the purposes of the present invention, it is believed that any of the various R gene family members may be successfully employed (e.g., P, S, Lc, etc.). However, the most preferred will generally be Sn (particularly Sn:bol3). Sn is a dominant member of the R gene complex and is functionally similar to the R and B loci in that Sn controls the tissue specific deposition of anthocyanin pigments in certain seedling and plant cells, therefore, its phenotype is similar to R.

Another screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It also is envisioned that this system may be developed for populational screening for bioluminescence, such as on tissue culture plates, or even for whole plant screening. The gene which encodes green fluorescent protein (GFP) is contemplated as a particularly useful reporter gene (Sheen et al., 1995; Haseloff et al., 1997; Reichel et al., 1996; Tian et al., 1997; WO 97/41228). Expression of green fluorescent protein may be visualized in a cell or plant as fluorescence following illumination by particular wavelengths of light. Where use of a screenable marker gene such as lux or GFP is desired, the inventors contemplated that benefit may be realized by creating a gene fusion between the screenable marker gene and a selectable marker gene, for example, a GFP-NPTII gene fusion. This could allow, for example, selection of transformed cells followed by screening of transgenic plants or seeds.

II. Exogenous Genes for Modification of Plant Phenotypes

A particularly important advance of the present invention is that it provides methods for the efficient expression of selected genes in plant cells. The choice of a selected gene for expression in a plant host cell in accordance with the invention will depend on the purpose of the transformation. One of the major purposes of transformation of crop plants is to add commercially desirable, agronomically important traits to the plant. Such traits include, but are not limited to, herbicide resistance or tolerance; insect resistance or tolerance; disease resistance or tolerance (viral, bacterial, fungal, nematode); stress tolerance and/or resistance, as exemplified by resistance or tolerance to drought, heat, chilling, freezing, excessive moisture, salt stress and oxidative stress; increased yields; food content and makeup; physical appearance; male sterility; drydown; standability; prolificacy; starch quantity and quality; oil quantity and quality; protein quality and quantity; amino acid composition; and the like.

In certain embodiments of the invention, transformation of a recipient cell may be carried out with more than one exogenous (selected) gene. As used herein, an “exogenous gene” or “selected gene” is a gene not normally found in the host genome in an identical context. By this, it is meant that the gene may be isolated from a different species than that of the host genome, or alternatively, isolated from the host genome, but is operably linked to one or more regulatory regions which differ from those found in the unaltered, native gene. Two or more exogenous genes also can be supplied in a single transformation event using either distinct transgene-encoding vectors, or using a single vector incorporating two or more gene coding sequences. For example, plasmids bearing the bar and aroA expression units in either convergent, divergent, or colinear orientation, are considered to be particularly useful. Further preferred combinations are those of an insect resistance gene, such as a Bt gene, along with a protease inhibitor gene such as pinII, or the use of bar in combination with either of the above genes. Of course, any two or more transgenes of any description, such as those conferring herbicide, insect, disease (viral, bacterial, fungal, nematode) or drought resistance, male sterility, drydown, standability, prolificacy, starch properties, oil quantity and quality, or those increasing yield or nutritional quality may be employed as desired.

A. Herbicide Resistance

The genes encoding phosphinothricin acetyltransferase (bar and pat), glyphosate tolerant EPSP synthase genes, the glyphosate degradative enzyme gene gox encoding glyphosate oxidoreductase, deh (encoding a dehalogenase enzyme that inactivates dalapon), herbicide resistant (e.g., sulfonylurea and imidazolinone) acetolactate synthase, and bxn genes (encoding a nitrilase enzyme that degrades bromoxynil) are examples of herbicide resistant genes for use in transformation. The bar and pat genes code for an enzyme, phosphinothricin acetyltransferase (PAT), which inactivates the herbicide phosphinothricin and prevents this compound from inhibiting glutamine synthetase enzymes. The enzyme 5-enolpyruvyishikimate 3-phosphate synthase (EPSP Synthase), is normally inhibited by the herbicide N-(phosphonomethyl)glycine (glyphosate). However, genes are known that encode glyphosate-resistant EPSP synthase enzymes. These genes are contemplated as particularly useful for plant transformation. The deh gene encodes the enzyme dalapon dehalogenase and confers resistance to the herbicide dalapon. The bxn gene codes for a specific nitrilase enzyme that converts bromoxynil to a non-herbicidal degradation product.

B. Insect Resistance

Potential insect resistance genes that can be introduced include Bacillus thuringiensis crystal toxin genes or Bt genes (Watrud et al., 1985). Bt genes may provide resistance to lepidopteran or coleopteran pests such as European Corn Borer (ECB). Preferred Bt toxin genes for use in such embodiments include the CryIA(b) and CryIA(c) genes. Endotoxin genes from other species of B. thuringiensis which affect insect growth or development also may be employed in this regard.

It is contemplated that preferred Bt genes for use in the transformation protocols disclosed herein will be those in which the coding sequence has been modified to effect increased expression in plants, and more particularly, in maize. Means for preparing synthetic genes are well known in the art and are disclosed in, for example, U.S. Pat. No. 5,500,365 and U.S. Pat. No. 5,689,052, each of the disclosures of which are specifically incorporated herein by reference in their entirety. Examples of such modified Bt toxin genes include a synthetic Bt CryIA(b) gene (Perlak et al., 1991), and the synthetic CryIA (c) gene termed 1800b (PCT Application WO 95/06128). Some examples of other Bt toxin genes known to those of skill in the art are given in Table 1 below.

TABLE 1
Bacillus thuringiensis δ-Endotoxin Genes^{a}

| New Nomenclature | Old Nomenclature | GenBank Accession |
|---|---|---|
| Cry1Aa | CryIA(a) | M11250 |
| Cry1Ab | CryIA(b) | M13898 |
| Cry1Ac | CryIA(c) | M11068 |
| Cry1Ad | CryIA(d) | M73250 |
| Cry1Ae | CryIA(e) | M65252 |
| Cry1Ba | CryIB | X06711 |
| Cry1Bb | ET5 | L32020 |
| Cry1Bc | PEG5 | Z46442 |
| Cry1Bd | CryE1 | U70726 |
| Cry1Ca | CryIC | X07518 |
| Cry1Cb | CryIC(b) | M97880 |
| Cry1Da | CryID | X54160 |
| Cry1Db | PrtB | Z22511 |
| Cry1Ea | CryIE | X53985 |
| Cry1Eb | CryIE(b) | M73253 |
| Cry1Fa | CryIF | M63897 |
| Cry1Fb | PrtD | Z22512 |
| Cry1Ga | PrtA | Z22510 |
| Cry1Gb | CryH2 | U70725 |
| Cry1Ha | PrtC | Z22513 |
| Cry1Hb | | U35780 |
| Cry1Ia | CryV | X62821 |
| Cry1Ib | CryV | U07642 |
| Cry1Ja | ET4 | L32019 |
| Cry1Jb | ET1 | U31527 |
| Cry1K | | U28801 |
| Cry2Aa | CryIIA | M31738 |
| Cry2Ab | CryIIB | M23724 |
| Cry2Ac | CryIIC | X57252 |
| Cry3A | CryIIIA | M22472 |
| Cry3Ba | CryIIIB | X17123 |
| Cry3Bb | CryIIIB2 | M89794 |
| Cry3C | CryIIID | X59797 |
| Cry4A | CryIVA | Y00423 |
| Cry4B | CryIVB | X07423 |
| Cry5Aa | CryVA(a) | L07025 |
| Cry5Ab | CryVA(b) | L07026 |
| Cry6A | CryVIA | L07022 |
| Cry6B | CryVIB | L07024 |
| Cry7Aa | CryIIIC | M64478 |
| Cry7Ab | CryIIICb | U04367 |
| Cry8A | CryIIIE | U04364 |
| Cry8B | CryIIIG | U04365 |
| Cry8C | CryIIIF | U04366 |
| Cry9A | CryIG | X58120 |
| Cry9B | CryIX | X75019 |
| Cry9C | CryIH | Z37527 |
| Cry10A | CryIVC | M12662 |
| Cry11A | CryIVD | M31737 |
| Cry11B | Jeg80 | X86902 |
| Cry12A | CryVB | L07027 |
| Cry13A | CryVC | L07023 |
| Cry14A | CryVD | U13955 |
| Cry15A | 34kDa | M76442 |
| Cry16A | cbm71 | X94146 |
| Cry17A | cbm71 | X99478 |
| Cry18A | CryBP1 | X99049 |
| Cry19A | Jeg65 | Y08920 |
| Cyt1Aa | CytA | X03182 |
| Cyt1Ab | CytM | X98793 |
| Cyt2A | CytB | Z14147 |
| Cyt2B | CytB | U52043 |

^{a}Adapted from: http://epunix.biols.susx.ac.uk/Home/Neil_Crickmore/Bt/index.html

Protease inhibitors also may provide insect resistance (Johnson et al., 1989), and thus will have utility in plant transformation. The use of a protease inhibitor II gene, pinII, from tomato or potato is envisioned to be particularly useful. Even more advantageous is the use of a pinII gene in combination with a Bt toxin gene, the combined effect of which has been discovered to produce synergistic insecticidal activity. Other genes which encode inhibitors of the insect's digestive system, or those that encode enzymes or co-factors that facilitate the production of inhibitors, also may be useful. This group may be exemplified by oryzacystatin and amylase inhibitors such as those from wheat and barley.

Also, genes encoding lectins may confer additional or alternative insecticide properties. Lectins (originally termed phytohemagglutinins) are multivalent carbohydrate-binding proteins which have the ability to agglutinate red blood cells from a range of species. Lectins have been identified recently as insecticidal agents with activity against weevils, ECB and rootworm (Murdock et al., 1990; Czapla & Lang, 1990). Lectin genes contemplated to be useful include, for example, barley and wheat germ agglutinin (WGA) and rice lectins (Gatehouse et al., 1984), with WGA being preferred.

Genes controlling the production of large or small polypeptides active against insects when introduced into the insect pests, such as, e.g., lytic peptides, peptide hormones and toxins and venoms, form another aspect of the invention. For example, it is contemplated that the expression of juvenile hormone esterase, directed towards specific insect pests, also may result in insecticidal activity, or perhaps cause cessation of metamorphosis (Hammock et al., 1990).

Transgenic plants expressing genes which encode enzymes that affect the integrity of the insect cuticle form yet another aspect of the invention. Such genes include those encoding, e.g., chitinase, proteases, lipases and also genes for the production of nikkomycin, a compound that inhibits chitin synthesis, the introduction of any of which is contemplated to produce insect resistant plants. Genes that code for activities that affect insect molting, such as those affecting the production of ecdysteroid UDP-glucosyl transferase, also fall within the scope of the useful transgenes of the present invention.

Genes that code for enzymes that facilitate the production of compounds that reduce the nutritional quality of the host plant to insect pests also are encompassed by the present invention. It may be possible, for instance, to confer insecticidal activity on a plant by altering its sterol composition. Sterols are obtained by insects from their diet and are used for hormone synthesis and membrane stability. Therefore alterations in plant sterol composition by expression of novel genes, e.g., those that directly promote the production of undesirable sterols or those that convert desirable sterols into undesirable forms, could have a negative effect on insect growth and/or development and hence endow the plant with insecticidal activity. Lipoxygenases are naturally occurring plant enzymes that have been shown to exhibit anti-nutritional effects on insects and to reduce the nutritional quality of their diet. Therefore, further embodiments of the invention concern transgenic plants with enhanced lipoxygenase activity which may be resistant to insect feeding.

Tripsacum dactyloides is a species of grass that is resistant to certain insects, including corn root worm. It is anticipated that genes encoding proteins that are toxic to insects or are involved in the biosynthesis of compounds toxic to insects will be isolated from Tripsacum and that these novel genes will be useful in conferring resistance to insects. It is known that the basis of insect resistance in Tripsacum is genetic, because said resistance has been transferred to Zea mays via sexual crosses (Branson and Guss, 1972). It further is anticipated that other cereal, monocot or dicot plant species may have genes encoding proteins that are toxic to insects which would be useful for producing insect resistant corn plants.

Further genes encoding proteins characterized as having potential insecticidal activity also may be used as transgenes in accordance herewith. Such genes include, for example, the cowpea trypsin inhibitor (CpTI; Hilder et al., 1987) which may be used as a rootworm deterrent; genes encoding avermectin (Avermectin and Abamectin., Campbell, W. C., Ed., 1989; Ikeda et al., 1987) which may prove particularly useful as a corn rootworm deterrent; ribosome inactivating protein genes; and even genes that regulate plant structures. Transgenic maize including anti-insect antibody genes and genes that code for enzymes that can convert a non-toxic insecticide (pro-insecticide) applied to the outside of the plant into an insecticide inside the plant also are contemplated.

C. Environment or Stress Resistance

Improvement of a plants ability to tolerate various environmental stresses such as, but not limited to, drought, excess moisture, chilling, freezing, high temperature, salt, and oxidative stress, also can be effected through expression of novel genes. It is proposed that benefits may be realized in terms of increased resistance to freezing temperatures through the introduction of an “antifreeze” protein such as that of the Winter Flounder (Cutler et al., 1989) or synthetic gene derivatives thereof. Improved chilling tolerance also may be conferred through increased expression of glycerol-3-phosphate acetyltransferase in chloroplasts (Wolter et al., 1992). Resistance to oxidative stress (often exacerbated by conditions such as chilling temperatures in combination with high light intensities) can be conferred by expression of superoxide dismutase (Gupta et al., 1993), and may be improved by glutathione reductase (Bowler et al., 1992). Such strategies may allow for tolerance to freezing in newly emerged fields as well as extending later maturity higher yielding varieties to earlier relative maturity zones.

It is contemplated that the expression of novel genes that favorably effect plant water content, total water potential, osmotic potential, and turgor will enhance the ability of the plant to tolerate drought. As used herein, the terms “drought resistance” and “drought tolerance” are used to refer to a plants increased resistance or tolerance to stress induced by a reduction in water availability, as compared to normal circumstances, and the ability of the plant to function and survive in lower-water environments. In this aspect of the invention it is proposed, for example, that the expression of genes encoding for the biosynthesis of osmotically-active solutes, such as polyol compounds, may impart protection against drought. Within this class are genes encoding for mannitol-L-phosphate dehydrogenase (Lee and Saier, 1982) and trehalose-6-phosphate synthase (Kaasen et al., 1992). Through the subsequent action of native phosphatases in the cell or by the introduction and coexpression of a specific phosphatase, these introduced genes will result in the accumulation of either mannitol or trehalose, respectively, both of which have been well documented as protective compounds able to mitigate the effects of stress. Mannitol accumulation in transgenic tobacco has been verified and preliminary results indicate that plants expressing high levels of this metabolite are able to tolerate an applied osmotic stress (Tarczynski et al., 1992, 1993).

Similarly, the efficacy of other metabolites in protecting either enzyme function (e.g., alanopine or propionic acid) or membrane integrity (e.g., alanopine) has been documented (Loomis et al., 1989), and therefore expression of genes encoding for the biosynthesis of these compounds might confer drought resistance in a manner similar to or complimentary to mannitol. Other examples of naturally occurring metabolites that are osmotically active and/or provide some direct protective effect during drought and/or desiccation include fructose, erythritol (Coxson et al., 1992), sorbitol, dulcitol (Karsten et al., 1992), glucosylglycerol (Reed et al., 1984; Erdmann et al., 1992), sucrose, stachyose (Koster and Leopold, 1988; Blackman et al., 1992), raffinose (Bernal-Lugo and Leopold, 1992), proline (Rensburg et al., 1993), glycine betaine, ononitol and pinitol (Vernon and Bohnert, 1992). Continued canopy growth and increased reproductive fitness during times of stress will be augmented by introduction and expression of genes such as those controlling the osmotically active compounds discussed above and other such compounds. Currently preferred genes which promote the synthesis of an osmotically active polyol compound are genes which encode the enzymes mannitol-1-phosphate dehydrogenase, trehalose-6-phosphate synthase and myomositol 0-methyltransferase.

It is contemplated that the expression of specific proteins also may increase drought tolerance. Three classes of Late Embryogenic Proteins have been assigned based on structural similarities (see Dure et al., 1989). All three classes of LEAs have been demonstrated in maturing (i.e. desiccating) seeds. Within these 3 types of LEA proteins, the Type-II (dehydrin-type) have generally been implicated in drought and/or desiccation tolerance in vegetative plant parts (i.e. Mundy and Chua, 1988; Piatkowski et al., 1990; Yamaguchi-Shinozaki et al., 1992). Recently, expression of a Type-III LEA (HVA-1) in tobacco was found to influence plant height, maturity and drought tolerance (Fitzpatrick, 1993). In rice, expression of the HVA-1 gene influenced tolerance to water deficit and salinity (Xu et al., 1996). Expression of structural genes from all three LEA groups may therefore confer drought tolerance. Other types of proteins induced during water stress include thiol proteases, aldolases and transmembrane transporters (Guerrero et al., 1990), which may confer various protective and/or repair-type functions during drought stress. It also is contemplated that genes that effect lipid biosynthesis and hence membrane composition might also be useful in conferring drought resistance on the plant.

Many of these genes for improving drought resistance have complementary modes of action. Thus, it is envisaged that combinations of these genes might have additive and/or synergistic effects in improving drought resistance in crop plants such as, for example, corn. Many of these genes also improve freezing tolerance (or resistance); the physical stresses incurred during freezing and drought are similar in nature and may be mitigated in similar fashion. Benefit may be conferred via constitutive expression of these genes, but the preferred means of expressing these novel genes may be through the use of a turgor-induced promoter (such as the promoters for the turgor-induced genes described in Guerrero et al., 1990 and Shagan et al., 1993 which are incorporated herein by reference). Spatial and temporal expression patterns of these genes may enable plants to better withstand stress.

It is proposed that expression of genes that are involved with specific morphological traits that allow for increased water extractions from drying soil would be of benefit. For example, introduction and expression of genes that alter root characteristics may enhance water uptake. It also is contemplated that expression of genes that enhance reproductive fitness during times of stress would be of significant value. For example, expression of genes that improve the synchrony of pollen shed and receptiveness of the female flower parts, i.e., silks, would be of benefit. In addition it is proposed that expression of genes that minimize kernel abortion during times of stress would increase the amount of grain to be harvested and hence be of value.

Given the overall role of water in determining yield, it is contemplated that enabling corn and other crop plants to utilize water more efficiently, through the introduction and expression of novel genes, will improve overall performance even when soil water availability is not limiting. By introducing genes that improve the ability of plants to maximize water usage across a full range of stresses relating to water availability, yield stability or consistency of yield performance may be realized.

D. Disease Resistance

It is proposed that increased resistance to diseases may be realized through introduction of genes into plants, for example, into monocot plants such as maize. It is possible to produce resistance to diseases caused by viruses, bacteria, fungi and nematodes. It also is contemplated that control of mycotoxin producing organisms may be realized through expression of introduced genes.

Resistance to viruses may be produced through expression of novel genes. For example, it has been demonstrated that expression of a viral coat protein in a transgenic plant can impart resistance to infection of the plant by that virus and perhaps other closely related viruses (Cuozzo et al., 1988, Hemenway et al., 1988, Abel et al., 1986). It is contemplated that expression of antisense genes targeted at essential viral functions also may impart resistance to viruses. For example, an antisense gene targeted at the gene responsible for replication of viral nucleic acid may inhibit replication and lead to resistance to the virus. It is believed that interference with other viral functions through the use of antisense genes also may increase resistance to viruses. Similarly, ribozymes could be used in this context. Further, it is proposed that it may be possible to achieve resistance to viruses through other approaches, including, but not limited to the use of satellite viruses. Examples of viral and viral-like diseases, for which one could introduce resistance to in a transgenic plant in accordance with the instant invention, are listed below, in Table 2.

TABLE 2
Plant Virus and Virus-like Diseases

| DISEASE | CAUSATIVE AGENT |
|---|---|
| American wheat striate (wheat striate mosaic) | American wheat striate mosaic virus mosaic |
| | (AWSMV) |
| Barley stripe mosaic | Barley stripe mosaic virus (BSMV) |
| Barley yellow dwarf | Barley yellow dwarf virus (BYDV) |
| Brome mosaic | Brome mosaic virus (BMV) |
| Cereal chlorotic mottle* | Cereal chlorotic mottle virus (CCMV) |
| Corn chlorotic vein banding (Brazilian maize | Corn chlorotic vein banding virus (CCVBV) |
| mosaic)¹ | |
| Corn lethal necrosis | Virus complex (Maize chlorotic mottle |
| | virus[MCMV] and Maize dwarf mosaic virus |
| | [MDMV] A or B or Wheat streak mosaic |
| | virus[WSMV]) |
| Cucumber mosaic | Cucumber mosaic virus (CMV) |
| Cynodon chlorotic streak*^{,1} | Cynodon chlorotic streak virus (CCSV) |
| Johnsongrass mosaic | Johnsongrass mosaic virus (JGMV) |
| Maize bushy stunt | Mycoplasma-like organism (MLO) associated |
| Maize chlorotic dwarf | Maize chlorotic dwarf virus (MCDV) |
| Maize chlorotic mottle | Maize chlorotic mottle virus (MCMV) |
| Maize dwarf mosaic | Maize dwarf mosaic virus (MDMV) strains A, |
| | D, E and F |
| Maize leaf fleck | Maize leaf fleck virus (MLFV) |
| Maize line* | Maize line virus (MLV) |
| Maize mosaic (corn leaf stripe, enanismo | Maize mosaic virus (MMV) |
| rayado) | |
| Maize mottle and chlorotic stunt¹ | Maize mottle and chlorotic stunt virus* |
| Maize pellucid ringspot* | Maize pellucid ringspot virus (MPRV) |
| Maize raya gruesa*^{,1} | Maize raya gruesa virus (MRGV) |
| maize rayado fino* (fine striping disease) | Maize rayado fino virus (MRFV) |
| Maize red leaf and red stripe* | Mollicute? |
| Maize red stripe* | Maize red stripe virus (MRSV) |
| Maize ring mottle* | Maize ring mottle virus (MRMV) |
| Maize rio IV* | Maize rio cuarto virus (MRCV) |
| Maize rough dwarf* (nanismo ruvido) | Maize rough dwarf virus (MRDV) (= Cereal |
| | tillering disease virus*) |
| Maize sterile stunt* | Maize sterile stunt virus (strains of barley |
| | yellow striate virus) |
| Maize streak* | Maize streak virus (MSV) |
| Maize stripe (maize chlorotic stripe, maize | Maize stripe virus |
| hoja blanca) | |
| Maize stunting*^{,1} | Maize stunting virus |
| Maize tassel abortion* | Maize tassel abortion virus (MTAV) |
| Maize vein enation* | Maize vein enation virus (MVEV) |
| Maize wallaby ear* | Maize wallaby ear virus (MWEV) |
| Maize white leaf* | Maize white leaf virus |
| Maize white line mosaic | Maize white line mosaic virus (MWLMV) |
| Millet red leaf* | Millet red leaf virus (MRLV) |
| Northern cereal mosaic* | Northern cereal mosaic virus (NCMV) |
| Oat pseudorosette* (zakuklivanie) | Oat pseudorosette virus |
| Oat sterile dwarf* | Oat sterile dwarf virus (OSDV) |
| Rice black-streaked dwarf* | Rice black-streaked dwarf virus (RBSDV) |
| Rice stripe* | Rice stripe virus (RSV) |
| Sorghum mosaic | Sorghum mosaic virus (SrMV), formerly |
| | sugarcane mosaic virus (SCMV) strains H, I |
| | and M |
| Sugarcane Fiji disease* | Sugarcane Fiji disease virus (FDV) |
| Sugarcane mosaic | Sugarcane mosaic virus (SCMV) strains A, B, |
| | D, E, SC, BC, Sabi and MB (formerly MDMV- |
| | B) |
| Vein enation*^{,1} | Virus ? |
| Wheat spot mosaic¹ | Wheat spot mosaic virus (WSMV) |

*Not known to occur naturally on corn in the United States.
¹Minor viral disease.

It is proposed that increased resistance to diseases caused by bacteria and fungi also may be realized through introduction of novel genes. It is contemplated that genes encoding so-called “peptide antibiotics,” pathogenesis related (PR) proteins, toxin resistance, and proteins affecting host-pathogen interactions such as morphological characteristics will be useful. Peptide antibiotics are polypeptide sequences which are inhibitory to growth of bacteria and other microorganisms. For example, the classes of peptides referred to as cecropins and magainins inhibit growth of many species of bacteria and fungi. It is proposed that expression of PR proteins in monocot plants such as maize may be useful in conferring resistance to bacterial disease. These genes are induced following pathogen attack on a host plant and have been divided into at least five classes of proteins (Bol, Linthorst, and Cornelissen, 1990). Included amongst the PR proteins are β-1,3-glucanases, chitinases, and osmotin and other proteins that are believed to function in plant resistance to disease organisms. Other genes have been identified that have antifungal properties, e.g., UDA (stinging nettle lectin) and hevein (Broakaert et al., 1989; Barkai-Golan et al., 1978). It is known that certain plant diseases are caused by the production of phytotoxins. It is proposed that resistance to these diseases would be achieved through expression of a novel gene that encodes an enzyme capable of degrading or otherwise inactivating the phytotoxin. It also is contemplated that expression of novel genes that alter the interactions between the host plant and pathogen may be useful in reducing the ability of the disease organism to invade the tissues of the host plant, e.g., an increase in the waxiness of the leaf cuticle or other morphological characteristics. Examples of bacterial and fungal diseases, including downy mildews, for which one could introduce resistance to in a transgenic plant in accordance with the instant invention, are listed below, in Tables 3, 4 and 5.

TABLE 3
Plant Bacterial Diseases

| DISEASE | CAUSATIVE AGENT |
|---|---|
| Bacterial leaf blight and stalk rot | Pseudomonas avenae esp. avenae |
| Bacterial leaf spot | Xanthomonas campestris pv. holcicola |
| Bacterial stalk rot | Enterobacter dissolvens = Erwinia dissolvens |
| Bacterial stalk and top rot | Erwinia carotovora subsp. carotovora, |
| | Erwinia chrysanthemi pv. zeae |
| Bacterial stripe | Pseudomonas andropogonis |
| Chocolate spot | Pseudomonas syringae pv. coronafaciens |
| Goss's bacterial wilt and blight (leaf freckles | Clavibacter michiganensis subsp. nebraskensis |
| and wilt) | = Corynebacterium michiganense pv. |
| | nebraskense |
| Holcus spot | Pseudomonas syringae pv. syringae |
| Purple leaf sheath | Hemiparasitic bacteria + (See under Fungi) |
| Seed rot-seedling blight | Bacillus subtilis |
| Stewart's disease (bacterial wilt) | Pantoea stewartii = Erwinia stewartii |
| Corn stunt (achapparramiento, maize stunt, | Spiroplasma kunkelii |
| Mesa Central or Rio Grande maize stunt) | |

TABLE 4
Plant Fungal Diseases

| DISEASE | PATHOGEN |
|---|---|
| Anthracnose leaf blight and anthracnose stalk | Colletotrichum graminicola (teleomorph: |
| rot | Glomerella graminicola Politis), Glomerella |
| | tucumanensis (anamorph: Glomerella falcatum |
| | Went) |
| Aspergillus ear and kernel rot | Aspergillus flavus Link: Fr. |
| Banded leaf and sheath spot* | Rhizoctonia solani Kuhn = Rhizoctonia |
| | microsclerotia J. Matz (teleomorph: |
| | Thanatephorus cucumeris) |
| Black bundle disease | Acremonium strictum W. Gams = |
| | Cephalosporium acremonium Auct. non Corda |
| Black kernel rot* | Lasiodiplodia theobromae = Botryodiplodia |
| | theobromae |
| Borde blanco* | Marasmiellus sp. |
| Brown spot (black spot, stalk rot) | Physoderma maydis |
| Cephalosporium kernel rot | Acremonium strictum = Cephalosporium |
| | acremonium |
| Charcoal rot | Macrophomina phaseolina |
| Corticium ear rot* | Thanatephorus cucumeris = Corticium sasakii |
| Curvularia leaf spot | Curvularia clavata, C. eragrostidis, = C. |
| | maculans (teleomorph: Cochliobolus |
| | eragrostidis), Curvularia inaequalis, C. |
| | intermedia (teleomorph: Cochliobolus |
| | intermedius), Curvularia lunata (teleomorph: |
| | Cochliobolus lunatus), Curvularia pallescens |
| | (teleomorph: Cochliobolus pallescens), |
| | Curvularia senegalensis, C. tuberculata |
| | (teleomorph: Cochliobolus tuberculatus) |
| Didymella leaf spot* | Didymella exitalis |
| Diplodia ear rot and stalk rot | Diplodia frumenti (teleomorph: |
| | Botryosphaeria festucae) |
| Diplodia ear rot, stalk rot, seed rot and seedling | Diplodia maydis = Stenocarpella maydis |
| blight | |
| Diplodia leaf spot or leaf streak | Stenocarpella macrospora = Diplodia |
| | macrospora |

*Not known to occur naturally on corn in the United States.

TABLE 5
Plant Downy Mildews

| DISEASE | CAUSATIVE AGENT |
|---|---|
| Brown stripe downy mildew* | Sclerophthora rayssiae var. zeae |
| Crazy top downy mildew | Sclerophthora macrospora = Sclerospora |
| | macrospora |
| Green ear downy mildew (graminicola downy | Sclerospora graminicola |
| mildew) | |
| Java downy mildew* | Peronosclerospora maydis = Sclerospora |
| | maydis |
| Philippine downy mildew* | Peronosclerospora philippinensis = |
| | Sclerospora philippinensis |
| Sorghum downy mildew | Peronosclerospora sorghi = Sclerospora |
| | sorghi |
| Spontaneum downy mildew* | Peronosclerospora spontanea = Sclerospora |
| | spontanea |
| Sugarcane downy mildew* | Peronosclerospora sacchari = Sclerospora |
| | sacchari |
| Dry ear rot (cob, kernel and stalk rot) | Nigrospora oryzae (teleomorph: Khuskia |
| | oryzae) |
| Ear rots, minor | Alternaria alternata = A. tenuis, Aspergillus |
| | glaucus, A. niger, Aspergillus spp., Botrytis |
| | cinerea (teleomorph: Botryotinia fuckeliana), |
| | Cunninghamella sp., Curvularia pallescens, |
| | Doratomyces stemonitis = Cephalotrichum |
| | stemonitis, Fusarium culmorum, Gonatobotrys |
| | simplex, Pithomyces maydicus, Rhizopus |
| | microsporus Tiegh., R. stolonifer = R. |
| | nigricans, Scopulariopsis brumptii. |
| Ergot* (horse's tooth, diente de caballo) | Claviceps gigantea (anamorph: Sphacelia sp.) |
| Eyespot | Aureobasidium zeae = Kabatiella zeae |
| Fusarium ear and stalk rot | Fusarium subglutinans = F. moniliforme var. |
| | subglutinans |
| Fusarium kernel, root and stalk rot, seed rot | Fusarium moniliforme (teleomorph: Gibberella |
| and seedling blight | fujikuroi) |
| Fusarium stalk rot, seedling root rot | Fusarium avenaceum (teleomorph: Gibberella |
| | avenacea) |
| Gibberella ear and stalk rot | Gibberella zeae (anamorph: Fusarium |
| | graminearum) |
| Gray ear rot | Botryosphaeria zeae = Physalospora zeae |
| | (anamorph: Macrophoma zeae) |
| Gray leaf spot (Cercospora leaf spot) | Cercospora sorghi = C. sorghi var. maydis, C. |
| | zeae-maydis |
| Helminthosporium root rot | Exserohilum pedicellatum = Helminthosporium |
| | pedicellatum (teleomorph: Setosphaeria |
| | pedicellata) |
| Hormodendrum ear rot (Cladosporium rot) | Cladosporium cladosporioides = |
| | Hormodendrum cladosporioides, C. herbarum |
| | (teleomorph: Mycosphaerella tassiana) |
| Hyalothyridium leaf spot* | Hyalothyridium maydis |
| Late wilt* | Cephalosporium maydis |
| Leaf spots, minor | Alternaria alternata, Ascochyta maydis, |
| | A. tritici, A. zeicola, Bipolaris victoriae = |
| | Helminthosporium victoriae (teleomorph: |
| | Cochliobolus victoriae), C. sativus (anamorph: |
| | Bipolaris sorokiniana = H. sorokinianum = H. |
| | sativum), Epicoccum nigrum, Exserohilum |
| | prolatum = Drechslera prolata (teleomorph: |
| | Setosphaeria prolata) Graphium penicillioides, |
| | Leptosphaeria maydis, Leptothyrium zeae, |
| | Ophiosphaerella herpotricha, (anamorph: |
| | Scolecosporiella sp.), Paraphaeosphaeria |
| | michotii, Phoma sp., Septoria zeae, S. zeicola, |
| | S. zeina |
| Northern corn leaf blight (white blast, crown | Setosphaeria turcica (anamorph: Exserohilum |
| stalk rot, stripe) | turcicum = Helminthosporium turcicum) |
| Northern corn leaf spot, Helminthosporium ear | Cochliobolus carbonum (anamorph: Bipolaris |
| rot (race 1) | zeicola = Helminthosporium carbonum) |
| Penicillium ear rot (blue eye, blue mold) | Penicillium spp., P. chrysogenum, P. |
| | expansum, P. oxalicum |
| Phaeocytostroma stalk rot and root rot | Phaeocytostroma ambiguum, = |
| | Phaeocytosporella zeae |
| Phaeosphaeria leaf spot* | Phaeosphaeria maydis = Sphaerulina maydis |
| Physalospora ear rot (Botryosphaeria ear rot) | Botryosphaeria festucae = Physalospora |
| | zeicola (anamorph: Diplodia frumenti) |
| Purple leaf sheath | Hemiparasitic bacteria and fungi |
| Pyrenochaeta stalk rot and root rot | Phoma terrestris = Pyrenochaeta terrestris |
| Pythium root rot | Pythium spp., P. arrhenomanes, P. |
| | graminicola |
| Pythium stalk rot | Pythium aphanidermatum = P. butleri L. |
| Red kernel disease (ear mold, leaf and seed rot) | Epicoccum nigrum |
| Rhizoctonia ear rot (sclerotial rot) | Rhizoctonia zeae (teleomorph: Waitea |
| | circinata) |
| Rhizoctonia root rot and stalk rot | Rhizoctonia solani, Rhizoctonia zeae |
| Root rots, minor | Alternaria alternata, Cercospora sorghi, |
| | Dictochaeta fertilis, Fusarium acuminatum |
| | (teleomorph: Gibberella acuminata), F. |
| | equiseti (teleomorph: G. intricans), F. |
| | oxysporum, F. pallidoroseum, F. poae, F. |
| | roseum, G. cyanogena, (anamorph: F. |
| | sulphureum), Microdochium bolleyi, Mucor |
| | sp., Periconia circinata, Phytophthora |
| | cactorum, P. drechsleri, P. nicotianae var. |
| | parasitica, Rhizopus arrhizus |
| Rostratum leaf spot (Helminthosporium leaf | Setosphaeria rostrata, (anamorph: |
| disease, ear and stalk rot) | Exserohilum rostratum = Helminthosporium |
| | rostratum) |
| Rust, common corn | Puccinia sorghi |
| Rust, southern corn | Puccinia polysora |
| Rust, tropical corn | Physopella pallescens, P. zeae = Angiopsora |
| | zeae |
| Sclerotium ear rot* (southern blight) | Sclerotium rolfsii Sacc. (teleomorph: Athelia |
| | rolfsii) |
| Seed rot-seedling blight | Bipolaris sorokiniana, B. zeicola = |
| | Helminthosporium carbonum, Diplodia |
| | maydis, Exserohilum pedicillatum, |
| | Exserohilum turcicum = Helminthosporium |
| | turcicum, Fusarium avenaceum, F. culmorum, |
| | F. moniliforme, Gibberella zeae (anamorph: F. |
| | graminearum), Macrophomina phaseolina, |
| | Penicillium spp., Phomopsis sp., Pythium spp., |
| | Rhizoctonia solani, R. zeae, Sclerotium rolfsii, |
| | Spicaria sp. |
| Selenophoma leaf spot* | Selenophoma sp. |
| Sheath rot | Gaeumannomyces graminis |
| Shuck rot | Myrothecium gramineum |
| Silage mold | Monascus purpureus, M. ruber |
| Smut, common | Ustilago zeae = U. maydis) |
| Smut, false | Ustilaginoidea virens |
| Smut, head | Sphacelotheca reiliana = Sporisorium holci- |
| | sorghi |
| Southern corn leaf blight and stalk rot | Cochliobolus heterostrophus (anamorph: |
| | Bipolaris maydis = Helminthosporium maydis) |
| Southern leaf spot | Stenocarpella macrospora = Diplodia |
| | macrospora |
| Stalk rots, minor | Cercospora sorghi, Fusarium episphaeria, F. |
| | merismoides, F. oxysporum Schlechtend, F. |
| | poae, F. roseum, F. solani (teleomorph: |
| | Nectria haematococca), F. tricinctum, |
| | Mariannaea elegans, Mucor sp., |
| | Rhopographus zeae, Spicaria sp. |
| Storage rots | Aspergillus spp., Penicillium spp. and other |
| | fungi |
| Tar spot* | Phyllachora maydis |
| Trichoderma ear rot and root rot | Trichoderma viride = T. lignorum teleomorph: |
| | Hypocrea sp. |
| White ear rot, root and stalk rot | Stenocarpella maydis = Diplodia zeae |
| Yellow leaf blight | Ascochyta ischaemi, Phyllosticta maydis |
| | (teleomorph: Mycosphaerella zeae-maydis) |
| Zonate leaf spot | Gloeocercospora sorghi |

*Not known to occur naturally on corn in the United States.

Plant parasitic nematodes are a cause of disease in many plants, for example, maize. It is proposed that it would be possible to make plants resistant to these organisms through the expression of novel genes. It is anticipated that control of nematode infestations would be accomplished by altering the ability of the nematode to recognize or attach to a host plant and/or enabling the plant to produce nematicidal compounds, including but not limited to proteins. Examples of nematode-associated plant diseases, for which one could introduce resistance to in a transgenic plant in accordance with the invention are given below, in Table 6.

TABLE 6
Parasitic Nematodes

| DISEASE | PATHOGEN |
|---|---|
| Awl | Dolichodorus spp., D. heterocephalus |
| Bulb and stem (Europe) | Ditylenchus dipsaci |
| Burrowing | Radopholus similis |
| Cyst | Heterodera avenae, H. zeae, Punctodera |
| | chalcoensis |
| Dagger | Xiphinema spp., X. americanum, X. |
| | mediterraneum |
| False root-knot | Nacobbus dorsalis |
| Lance, Columbia | Hoplolaimus columbus |
| Lance | Hoplolaimus spp., H. galeatus |
| Lesion | Pratylenchus spp., P. brachyurus, P. crenatus, |
| | P. hexincisus, P. neglectus, P. penetrans, P. |
| | scribneri, P. thornei, P. zeae |
| Needle | Longidorus spp., L. breviannulatus |
| Ring | Criconemella spp., C. ornata |
| Root-knot | Meloidogyne spp., M. chitwoodi, M. incognita, |
| | M. javanica |
| Spiral | Helicotylenchus spp. |
| Sting | Belonolaimus spp., B. longicaudatus |
| Stubby-root | Paratrichodorus spp., P. christiei, P. minor, |
| | Quinisulcius acutus, Trichodorus spp. |
| Stunt | Tylenchorhynchus dubius |

Mycotoxin Reduction/Elimination

Production of mycotoxins, including aflatoxin and fumonisin, by fungi associated with monocot plants such as maize is a significant factor in rendering the grain not useful. These fungal organisms do not cause disease symptoms and/or interfere with the growth of the plant, but they produce chemicals (mycotoxins) that are toxic to animals. It is contemplated that inhibition of the growth of these fungi would reduce the synthesis of these toxic substances and therefore reduce grain losses due to mycotoxin contamination. It also is proposed that it may be possible to introduce novel genes into monocot plants such as maize that would inhibit synthesis of the mycotoxin without interfering with fungal growth. Further, it is contemplated that expression of a novel gene which encodes an enzyme capable of rendering the mycotoxin nontoxic would be useful in order to achieve reduced mycotoxin contamination of grain. The result of any of the above mechanisms would be a reduced presence of mycotoxins on grain.

F. Grain Composition or Quality

Genes may be introduced into monocot plants, particularly commercially important cereals such as maize, to improve the grain for which the cereal is primarily grown. A wide range of novel transgenic plants produced in this manner may be envisioned depending on the particular end use of the grain.

The largest use of maize grain is for feed or food. Introduction of genes that alter the composition of the grain may greatly enhance the feed or food value. The primary components of maize grain are starch, protein, and oil. Each of these primary components of maize grain may be improved by altering its level or composition. Several examples may be mentioned for illustrative purposes, but in no way provide an exhaustive list of possibilities.

The protein of cereal grains including maize is suboptimal for feed and food purposes especially when fed to pigs, poultry, and humans. The protein is deficient in several amino acids that are essential in the diet of these species, requiring the addition of supplements to the grain. Limiting essential amino acids may include lysine, methionine, tryptophan, threonine, valine, arginine, and histidine. Some amino acids become limiting only after corn is supplemented with other inputs for feed formulations. For example, when corn is supplemented with soybean meal to meet lysine requirements methionine becomes limiting. The levels of these essential amino acids in seeds and grain may be elevated by mechanisms which include, but are not limited to, the introduction of genes to increase the biosynthesis of the amino acids, decrease the degradation of the amino acids, increase the storage of the amino acids in proteins, or increase transport of the amino acids to the seeds or grain.

One mechanism for increasing the biosynthesis of the amino acids is to introduce genes that deregulate the amino acid biosynthetic pathways such that the plant can no longer adequately control the levels that are produced. This may be done by deregulating or bypassing steps in the amino acid biosynthetic pathway which are normally regulated by levels of the amino acid end product of the pathway. Examples include the introduction of genes that encode deregulated versions of the enzymes aspartokinase or dihydrodipicolinic acid (DHDP)-synthase for increasing lysine and threonine production, and anthranilate synthase for increasing tryptophan production. Reduction of the catabolism of the amino acids may be accomplished by introduction of DNA sequences that reduce or eliminate the expression of genes encoding enzymes that catalyze steps in the catabolic pathways such as the enzyme lysine-ketoglutarate reductase. It is anticipated that it may be desirable to target expression of genes relating to amino acid biosynthesis to the endosperm or embryo of the seed. More preferably, the gene will be targeted to the embryo. It also will be preferable for genes encoding proteins involved in amino acid biosynthesis to target the protein to a plastid using a plastid transit peptide sequence.

The protein composition of the grain may be altered to improve the balance of amino acids in a variety of ways including elevating expression of native proteins, decreasing expression of those with poor composition, changing the composition of native proteins, or introducing genes encoding entirely new proteins possessing superior composition. Examples may include the introduction of DNA that decreases the expression of members of the zein family of storage proteins. This DNA may encode ribozymes or antisense sequences directed to impairing expression of zein proteins or expression of regulators of zein expression such as the opaque-2 gene product. It also is proposed that the protein composition of the grain may be modified through the phenomenon of co-suppression, i.e., inhibition of expression of an endogenous gene through the expression of an identical structural gene or gene fragment introduced through transformation (Goring et al., 1991). Additionally, the introduced DNA may encode enzymes which degrade zeins. The decreases in zein expression that are achieved may be accompanied by increases in proteins with more desirable amino acid composition or increases in other major seed constituents such as starch. Alternatively, a chimeric gene may be introduced that comprises a coding sequence for a native protein of adequate amino acid composition such as for one of the globulin proteins or 10 kD delta zein or 20 kD delta zein or 27 kD gamma zein of maize and a promoter or other regulatory sequence designed to elevate expression of said protein. The coding sequence of the gene may include additional or replacement codons for essential amino acids. Further, a coding sequence obtained from another species, or, a partially or completely synthetic sequence encoding a completely unique peptide sequence designed to enhance the amino acid composition of the seed may be employed. It is anticipated that it may be preferable to target expression of these transgenes encoding proteins with superior composition to the endosperm of the seed.

The introduction of genes that alter the oil content of the grain may be of value. Increases in oil content may result in increases in metabolizable-energy-content and density of the seeds for use in feed and food. The introduced genes may encode enzymes that remove or reduce rate-limitations or regulated steps in fatty acid or lipid biosynthesis. Such genes may include, but are not limited to, those that encode acetyl-CoA carboxylase, ACP-acyltransferase, β-ketoacyl-ACP synthase, plus other well known fatty acid biosynthetic activities. Other possibilities are genes that encode proteins that do not possess enzymatic activity such as acyl carrier protein. Genes may be introduced that alter the balance of fatty acids present in the oil providing a more healthful or nutritive feedstuff. The introduced DNA also may encode sequences that block expression of enzymes involved in fatty acid biosynthesis, altering the proportions of fatty acids present in the grain such as described below. Some other examples of genes specifically contemplated by the inventors for use in creating transgenic plants with altered oil composition traits include 2-acetyltransferase, oleosin, pyruvate dehydrogenase complex, acetyl CoA synthetase, ATP citrate lyase, ADP-glucose pyrophosphorylase and genes of the carnitine-CoA-acetyl-CoA shuffles. It is anticipated that expression of genes related to oil biosynthesis will be targeted to the plastid, using a plastid transit peptide sequence and preferably expressed in the seed embryo.

Genes may be introduced that enhance the nutritive value of the starch component of the grain, for example by increasing the degree of branching, resulting in improved utilization of the starch in cows by delaying its metabolism. It is anticipated that expression of genes related to starch biosynthesis will preferably be targeted to the endosperm of the seed.

Besides affecting the major constituents of the grain, genes may be introduced that affect a variety of other nutritive, processing, or other quality aspects of the grain as used for feed or food. For example, pigmentation of the grain may be increased or decreased. Enhancement and stability of yellow pigmentation is desirable in some animal feeds and may be achieved by introduction of genes that result in enhanced production of xanthophylls and carotenes by eliminating rate-limiting steps in their production. Such genes may encode altered forms of the enzymes phytoene synthase, phytoene desaturase, or lycopene synthase. Alternatively, unpigmented white corn is desirable for production of many food products and may be produced by the introduction of DNA which blocks or eliminates steps in pigment production pathways.

Most of the phosphorous content of the grain is in the form of phytate, a form of phosphate storage that is not metabolized by monogastric animals. Therefore, in order to increase the availability of seed phosphate, it is anticipated that one will desire to decrease the amount of phytate in seed and increase the amount of free phosphorous. Alternatively, one may express a gene in corn seed which will be activated, e.g., by pH, in the gastric system of a monogastric animal and will release phosphate from phytate, e.g., phytase.

Feed or food comprising primarily maize or other cereal grains possesses insufficient quantities of vitamins and must be supplemented to provide adequate nutritive value. introduction of genes that enhance vitamin biosynthesis in seeds may be envisioned including, for example, vitamins A, E, B₁₂, choline, and the like. Maize grain also does not possess sufficient mineral content for optimal nutritive value. Genes that affect the accumulation or availability of compounds containing phosphorus, sulfur, calcium, manganese, zinc, and iron among others would be valuable. An example may be the introduction of a gene that reduced phytic acid production or encoded the enzyme phytase which enhances phytic acid breakdown. These genes would increase levels of available phosphate in the diet, reducing the need for supplementation with mineral phosphate.

Numerous other examples of improvement of maize or other cereals for feed and food purposes might be described. The improvements may not even necessarily involve the grain, but may, for example, improve the value of the corn for silage. Introduction of DNA to accomplish this might include sequences that alter lignin production such as those that result in the “brown midrib” phenotype associated with superior feed value for cattle.

In addition to direct improvements in feed or food value, genes also may be introduced which improve the processing of corn and improve the value of the products resulting from the processing. The primary method of processing corn is via wetmilling. Maize may be improved though the expression of novel genes that increase the efficiency and reduce the cost of processing such as by decreasing steeping time.

Improving the value of wetmilling products may include altering the quantity or quality of starch, oil, corn gluten meal, or the components of corn gluten feed. Elevation of starch may be achieved through the identification and elimination of rate limiting steps in starch biosynthesis or by decreasing levels of the other components of the grain resulting in proportional increases in starch. An example of the former may be the introduction of genes encoding ADP-glucose pyrophosphorylase enzymes with altered regulatory activity or which are expressed at higher level. Examples of the latter may include selective inhibitors of, for example, protein or oil biosynthesis expressed during later stages of kernel development.

The properties of starch may be beneficially altered by changing the ratio of amylose to amylopectin, the size of the starch molecules, or their branching pattern. Through these changes a broad range of properties may be modified which include, but are not limited to, changes in gelatinization temperature, heat of gelatinization, clarity of films and pastes, rheological properties, and the like. To accomplish these changes in properties, genes that encode granule-bound or soluble starch synthase activity or branching enzyme activity may be introduced alone or combination. DNA such as antisense constructs also may be used to decrease levels of endogenous activity of these enzymes. The introduced genes or constructs may possess regulatory sequences that time their expression to specific intervals in starch biosynthesis and starch granule development. Furthermore, it may be worthwhile to introduce and express genes that result in the in vivo derivatization, or other modification, of the glucose moieties of the starch molecule. The covalent attachment of any molecule may be envisioned, limited only by the existence of enzymes that catalyze the derivatizations and the accessibility of appropriate substrates in the starch granule. Examples of important derivations may include the addition of functional groups such as amines, carboxyls, or phosphate groups which provide sites for subsequent in vitro derivatizations or affect starch properties through the introduction of ionic charges. Examples of other modifications may include direct changes of the glucose units such as loss of hydroxyl groups or their oxidation to aldehyde or carboxyl groups.

Oil is another product of wetmilling of corn, the value of which may be improved by introduction and expression of genes. The quantity of oil that can be extracted by wetmilling may be elevated by approaches as described for feed and food above. Oil properties also may be altered to improve its performance in the production and use of cooking oil, shortenings, lubricants or other oil-derived products or improvement of its health attributes when used in the food-related applications. Novel fatty acids also may be synthesized which upon extraction can serve as starting materials for chemical syntheses. The changes in oil properties may be achieved by altering the type, level, or lipid arrangement of the fatty acids present in the oil. This in turn may be accomplished by the addition of genes that encode enzymes that catalyze the synthesis of novel fatty acids and the lipids possessing them or by increasing levels of native fatty acids while possibly reducing levels of precursors. Alternatively, DNA sequences may be introduced which slow or block steps in fatty acid biosynthesis resulting in the increase in precursor fatty acid intermediates. Genes that might be added include desaturases, epoxidases, hydratases, dehydratases, and other enzymes that catalyze reactions involving fatty acid intermediates. Representative examples of catalytic steps that might be blocked include the desaturations from stearic to oleic acid and oleic to linolenic acid resulting in the respective accumulations of stearic and oleic acids. Another example is the blockage of elongation steps resulting in the accumulation of C₈ to C₁₂ saturated fatty acids.

Improvements in the other major corn wetmilling products, corn gluten meal and corn gluten feed, also may be achieved by the introduction of genes to obtain novel corn plants. Representative possibilities include but are not limited to those described above for improvement of food and feed value.

In addition, it may further be considered that the corn plant be used for the production or manufacturing of useful biological compounds that were either not produced at all, or not produced at the same level, in the corn plant previously. The novel corn plants producing these compounds are made possible by the introduction and expression of genes by corn transformation methods. The vast array of possibilities include but are not limited to any biological compound which is presently produced by any organism such as proteins, nucleic acids, primary and intermediary metabolites, carbohydrate polymers, etc. The compounds may be produced by the plant, extracted upon harvest and/or processing, and used for any presently recognized useful purpose such as pharmaceuticals, fragrances, and industrial enzymes to name a few.

Further possibilities to exemplify the range of grain traits or properties potentially encoded by introduced genes in transgenic plants include grain with less breakage susceptibility for export purposes or larger grit size when processed by dry milling through introduction of genes that enhance γ-zein synthesis, popcorn with improved popping quality and expansion volume through genes that increase pericarp thickness, corn with whiter grain for food uses though introduction of genes that effectively block expression of enzymes involved in pigment production pathways, and improved quality of alcoholic beverages or sweet corn through introduction of genes which affect flavor such as the shrunken 1 gene (encoding sucrose synthase) or shrunken 2 gene (encoding ADPG pyrophosphorylase) for sweet corn.

G. Plant Agronomic Characteristics

Two of the factors determining where crop plants can be grown are the average daily temperature during the growing season and the length of time between frosts. Within the areas where it is possible to grow a particular crop, there are varying limitations on the maximal time it is allowed to grow to maturity and be harvested. For example, maize to be grown in a particular area is selected for its ability to mature and dry down to harvestable moisture content within the required period of time with maximum possible yield. Therefore, corn of varying maturities is developed for different growing locations. Apart from the need to dry down sufficiently to permit harvest, it is desirable to have maximal drying take place in the field to minimize the amount of energy required for additional drying post-harvest. Also, the more readily the grain can dry down, the more time there is available for growth and kernel fill. It is considered that genes that influence maturity and/or dry down can be identified and introduced into corn or other plants using transformation techniques to create new varieties adapted to different growing locations or the same growing location, but having improved yield to moisture ratio at harvest. Expression of genes that are involved in regulation of plant development may be especially useful, e.g., the liguleless and rough sheath genes that have been identified in corn.

It is contemplated that genes may be introduced into plants that would improve standability and other plant growth characteristics. Expression of novel genes in maize which confer stronger stalks, improved root systems, or prevent or reduce ear droppage would be of great value to the farmer. It is proposed that introduction and expression of genes that increase the total amount of photo assimilate available by, for example, increasing light distribution and/or interception would be advantageous. In addition, the expression of genes that increase the efficiency of photosynthesis and/or the leaf canopy would further increase gains in productivity. It is contemplated that expression of a phytochrome gene in corn may be advantageous. Expression of such a gene may reduce apical dominance, confer semidwarfism on a plant, and increase shade tolerance U.S. Pat. No. 5,268,526). Such approaches would allow for increased plant populations in the field.

Delay of late season vegetative senescence would increase the flow of assimilate into the grain and thus increase yield. It is proposed that overexpression of genes within corn that are associated with “stay green” or the expression of any gene that delays senescence would be advantageous. For example, a nonyellowing mutant has been identified in Festuca pratensis (Davies et al., 1990). Expression of this gene as well as others may prevent premature breakdown of chlorophyll and thus maintain canopy function.

H. Nutrient Utilization

The ability to utilize available nutrients may be a limiting factor in growth of monocot plants such as maize. It is proposed that it would be possible to alter nutrient uptake, tolerate pH extremes, mobilization through the plant, storage pools, and availability for metabolic activities by the introduction of novel genes. These modifications would allow a plant such as maize to more efficiently utilize available nutrients. It is contemplated that an increase in the activity of, for example, an enzyme that is normally present in the plant and involved in nutrient utilization would increase the availability of a nutrient. An example of such an enzyme would be phytase. It further is contemplated that enhanced nitrogen utilization by a plant is desirable. Expression of a glutamate dehydrogenase gene in corn, e.g., E. coli gdhA genes, may lead to increased fixation of nitrogen in organic compounds. Furthermore, expression of gdhA in corn may lead to enhanced resistance to the herbicide glufosinate by incorporation of excess ammonia into glutamate, thereby detoxifying the ammonia. It also is contemplated that expression of a novel gene may make a nutrient source available that was previously not accessible, e.g., an enzyme that releases a component of nutrient value from a more complex molecule, perhaps a macromolecule.

I. Male Sterility

Male sterility is useful in the production of hybrid seed. It is proposed that male sterility may be produced through expression of novel genes. For example, it has been shown that expression of genes that encode proteins that interfere with development of the male inflorescence and/or gametophyte result in male sterility. Chimeric ribonuclease genes that express in the anthers of transgenic tobacco and oilseed rape have been demonstrated to lead to male sterility (Mariani et al., 1990).

A number of mutations were discovered in maize that confer cytoplasmic male sterility. One mutation in particular, referred to as T cytoplasm, also correlates with sensitivity to Southern corn leaf blight. A DNA sequence, designated TURF-13 (Levings, 1990), was identified that correlates with T cytoplasm. It is proposed that it would be possible through the introduction of TURF-13 via transformation, to separate male sterility from disease sensitivity. As it is necessary to be able to restore male fertility for breeding purposes and for grain production, it is proposed that genes encoding restoration of male fertility also may be introduced.

J. Negative Selectable Markers

Introduction of genes encoding traits that can be selected against may be useful for eliminating undesirable linked genes. It is contemplated that when two or more genes are introduced together by cotransformation that the genes will be linked together on the host chromosome. For example, a gene encoding a Bt gene that confers insect resistance on the plant may be introduced into a plant together with a bar gene that is useful as a selectable marker and confers resistance to the herbicide Liberty® on the plant. However, it may not be desirable to have an insect resistant plant that also is resistant to the herbicide Liberty®. It is proposed that one also could introduce an antisense bar gene that is expressed in those tissues where one does not want expression of the bar gene, e.g., in whole plant parts. Hence, although the bar gene is expressed and is useful as a selectable marker, it is not useful to confer herbicide resistance on the whole plant. The bar antisense gene is a negative selectable marker.

It also is contemplated that negative selection is necessary in order to screen a population of transformants for rare homologous recombinants generated through gene targeting. For example, a homologous recombinant may be identified through the inactivation of a gene that was previously expressed in that cell. The antisense gene to neomycin phosphotransferase II (NPT II) has been investigated as a negative selectable marker in tobacco (Nicotiana tabacum) and Arabidopsis thaliana (Xiang and Guerra, 1993). In this example, both sense and antisense NPT II genes are introduced into a plant through transformation and the resultant plants are sensitive to the antibiotic kanamycin. An introduced gene that integrates into the host cell chromosome at the site of the antisense NPT II gene, and inactivates the antisense gene, will make the plant resistant to kanamycin and other aminoglycoside antibiotics. Therefore, rare, site-specific recombinants may be identified by screening for antibiotic resistance. Similarly, any gene, native to the plant or introduced through transformation, that when inactivated confers resistance to a compound, may be useful as a negative selectable marker.

It is contemplated that negative selectable markers also may be useful in other ways. One application is to constrict transgenic lines in which one could select for transposition to unlinked sites. In the process of tagging it is most common for the transposable element to move to a genetically linked site on the same chromosome. A selectable marker for recovery of rare plants in which transposition has occurred to an unlinked locus would be useful. For example, the enzyme cytosine deaminase may be useful for this purpose (Stouggard, 1993). In the presence of this enzyme the compound 5-fluorocytosine is converted to 5-fluorouracil which is toxic to plant and animal cells. If a transposable element is linked to the gene for the enzyme cytosine deaminase, one may select for transposition to unlinked sites by selecting for transposition events in which the resultant plant is now resistant to 5-fluorocytosine. The parental plants and plants containing transpositions to linked sites will remain sensitive to 5-fluorocytosine. Resistance to 5-fluorocytosine is due to loss of the cytosine deaminase gene through genetic segregation of the transposable element and the cytosine deaminase gene. Other genes that encode proteins that render the plant sensitive to a certain compound also will be useful in this context. For example, T-DNA gene 2 from Agrobacterium tumefaciens encodes a protein that catalyzes the conversion of α-naphthalene acetamide (NAM) to α-naphthalene acetic acid BAA) renders plant cells sensitive to high concentrations of NAM (Depicker et al., 1988).

It also is contemplated that negative selectable markers may be useful in the construction of transposon tagging lines. For example, by marking an autonomous transposable element such as Ac, Master Mu, or En/Spn with a negative selectable marker, one could select for transformants in which the autonomous element is not stably integrated into the genome. It is proposed that this would be desirable, for example, when transient expression of the autonomous element is desired to activate in trans the transposition of a defective transposable element, such as Ds, but stable integration of the autonomous element is not desired. The presence of the autonomous element may not be desired in order to stabilize the defective element, i.e., prevent it from further transposing. However, it is proposed that if stable integration of an autonomous transposable element is desired in a plant the presence of a negative selectable marker may make it possible to eliminate the autonomous element during the breeding process.

K. Non-Protein-Expressing Sequences

DNA may be introduced into plants for the purpose of expressing RNA transcripts that function to affect plant phenotype yet are not translated into protein. Two examples are antisense RNA and RNA with ribozyme activity. Both may serve possible functions in reducing or eliminating expression of native or introduced plant genes. However, as detailed below, DNA need not be expressed to effect the phenotype of a plant.

1. Antisense RNA

Genes may be constructed or isolated, which when transcribed, produce antisense RNA that is complementary to all or part(s) of a targeted messenger RNA(s). The antisense RNA reduces production of the polypeptide product of the messenger RNA. The polypeptide product may be any protein encoded by the plant genome. The aforementioned genes will be referred to as antisense genes. An antisense gene may thus be introduced into a plant by transformation methods to produce a novel transgenic plant with reduced expression of a selected protein of interest. For example, the protein may be an enzyme that catalyzes a reaction in the plant. Reduction of the enzyme activity may reduce or eliminate products of the reaction which include any enzymatically synthesized compound in the plant such as fatty acids, amino acids, carbohydrates, nucleic acids and the like. Alternatively, the protein may be a storage protein, such as a zein, or a structural protein, the decreased expression of which may lead to changes in seed amino acid composition or plant morphological changes respectively. The possibilities cited above are provided only by way of example and do not represent the full range of applications.

2. Ribozymes

Genes also may be constructed or isolated, which when transcribed, produce RNA enzymes (ribozymes) which can act as endoribonucleases and catalyze the cleavage of RNA molecules with selected sequences. The cleavage of selected messenger RNAs can result in the reduced production of their encoded polypeptide products. These genes may be used to prepare novel transgenic plants which possess them. The transgenic plants may possess reduced levels of polypeptides including, but not limited to, the polypeptides cited above.

Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cech et al, 1981). For example, U.S. Pat. No. 5,354,855 reports that certain ribozymes can act as endonucleases with a sequence specificity greater than that of known ribonucleases and approaching that of the DNA restriction enzymes.

Several different ribozyme motifs have been described with RNA cleavage activity (Symons, 1992). Examples include sequences from the Group I self splicing introns including Tobacco Ringspot Virus (Prody et al., 1986), Avocado Sunblotch Viroid (Palukaitis et al., 1979; Symons, 1981), and Lucerne Transient Streak Virus (Forster and Symons, 1987). Sequences from these and related viruses are referred to as hammerhead ribozymes based on a predicted folded secondary structure.

Other suitable ribozymes include sequences from RNase P with RNA cleavage activity (Yuan et al., 1992, Yuan and Altman, 1994, U.S. Pat. Nos. 5,168,053 and 5,624,824), hairpin ribozyme structures (Berzal-Herranz et al., 1992; Chowrira et al., 1994) and Hepatitis Delta virus based ribozymes (U.S. Pat. No. 5,625,047). The general design and optimization of ribozyme directed RNA cleavage activity has been discussed in detail (Haseloff and Gerlach, 1988, Symons, 1992, Thompson et al., 1995).

The other variable on ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozyme, the cleavage site is a dinucleotide sequence on the target RNA is a uracil (U) followed by either an adenine, cytosine or uracil (A, C or U) (Perriman et al., 1992; Thompson et al., 1995). The frequency of this dinucleotide occurring in any given RNA is statistically 3 out of 16. Therefore, for a given target messenger RNA of 1000 bases, 187 dinucleotide cleavage sites are statistically possible.

Designing and testing ribozymes for efficient cleavage of a target RNA is a process well known to those skilled in the art. Examples of scientific methods for designing and testing ribozymes are described by Chowrira et al., (1994) and Lieber and Strauss (1995), each incorporated by reference. The identification of operative and preferred sequences for use in down regulating a given gene is simply a matter of preparing and testing a given sequence, and is a routinely practiced “screening” method known to those of skill in the art.

3. Induction of Gene Silencing

It also is possible that genes may be introduced to produce novel transgenic plants which have reduced expression of a native gene product by the mechanism of co-suppression. It has been demonstrated in tobacco, tomato, and petunia (Goring et al., 1991; Smith et al., 1990; Napoli et al., 1990; van der Krol et al., 1990) that expression of the sense transcript of a native gene will reduce or eliminate expression of the native gene in a manner similar to that observed for antisense genes. The introduced gene may encode all or part of the targeted native protein but its translation may not be required for reduction of levels of that native protein.

4. Non-RNA-Expressing Sequences

DNA elements including those of transposable elements such as Ds, Ac, or Mu, may be inserted into a gene to cause mutations. These DNA elements may be inserted in order to inactivate (or activate) a gene and thereby “tag” a particular trait. In this instance the transposable element does not cause instability of the tagged mutation, because the utility of the element does not depend on its ability to move in the genome. Once a desired trait is tagged, the introduced DNA sequence may be used to clone the corresponding gene, e.g., using the introduced DNA sequence as a PCR primer together with PCR gene cloning techniques (Shapiro, 1983; Dellaporta et al., 1988). Once identified, the entire gene(s) for the particular trait, including control or regulatory regions where desired, may be isolated, cloned and manipulated as desired. The utility of DNA elements introduced into an organism for purposes of gene tagging is independent of the DNA sequence and does not depend on any biological activity of the DNA sequence, i.e., transcription into RNA or translation into protein. The sole function of the DNA element is to disrupt the DNA sequence of a gene.

It is contemplated that unexpressed DNA sequences, including novel synthetic sequences, could be introduced into cells as proprietary “labels” of those cells and plants and seeds thereof. It would not be necessary for a label DNA element to disrupt the function of a gene endogenous to the host organism, as the sole function of this DNA would be to identify the origin of the organism. For example, one could introduce a unique DNA sequence into a plant and this DNA element would identify all cells, plants, and progeny of these cells as having arisen from that labeled source. It is proposed that inclusion of label DNAs would enable one to distinguish proprietary germplasm or germplasm derived from such, from unlabelled germplasm.

Another possible element which may be introduced is a matrix attachment region element (MAR), such as the chicken lysozyme A element (Stief, 1989), which can be positioned around an expressible gene of interest to effect an increase in overall expression of the gene and diminish position dependent effects upon incorporation into the plant genome (Stief et al., 1989; Phi-Van et al., 1990).

III. Assays for Transgene Expression

Assays may be employed with the instant invention for determination of the relative efficiency of transgene expression. For example, assays may be used to determine the efficacy of transgene expression with various promoters and selected genes. For plants, expression assays may comprise a system utilizing embryogenic or non-embryogenic cells, or alternatively, whole plants. An advantage of using cellular assays is that regeneration of large numbers of plants is not required. However, in some cases the systems are limited in that promoter activity in the non-regenerated cells may not always directly correlate with expression in a plant. Additionally, assays for tissue or developmental specific expression are generally not feasible.

The biological sample to be assayed may comprise nucleic acids isolated from the cells of any plant material according to standard methodologies (Sambrook et al., 1989). The nucleic acid may be genomic DNA or fractionated or whole cell RNA. Where RNA is used, it may be desired to convert the RNA to a complementary DNA. In one embodiment of the invention, the RNA is whole cell RNA; in another, it is polyA RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Following detection, one may compare the results seen in a given plant with a statistically significant reference group of non-transformed control plants. Typically, the non-transformed control plants will be of a genetic background similar to the transformed plants. In this way, it is possible to detect differences in the amount or kind of protein detected in various transformed plants. Alternatively, clonal cultures of cells, for example, callus or an immature embryo, may be compared to other cells samples.

As indicated, a variety of different assays are contemplated in the screening of cells or plants of the current invention and associated promoters. These techniques may in cases be used to detect for both the presence and expression of the particular genes as well as rearrangements that may have occurred in the gene construct. The techniques include but are not limited to, fluorescent in situ hybridization (FISH), direct DNA sequencing, pulsed field gel electrophoresis (PFGE) analysis, Southern or Northern blotting, single-stranded conformation analysis (SSCA), RNAse protection assay, allele-specific oligonucleotide (ASO), dot blot analysis, denaturing gradient gel electrophoresis, RFLP and PCR™-SSCP.

IV. Methods for Plant Transformation

A. Agrobacterium-Mediated Transformation

In one embodiment, the present invention deals with Agrobacterium-mediated transfer of genetic material. Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by Fraley et al., (1985), Rogers et al., (1987) and U.S. Pat. No. 5,563,055, specifically incorporated herein by reference in its entirety.

Agrobacterium tumefaciens harboring a tumor-inducing (Ti) plasmid is able to transfer specific DNA elements into higher eukaryotic genomes. The VirA/VirG two-component regulatory system of the Ti plasmid functions to perceive and respond to plant signals, such as acetosyringone, arabinose and low pH, and activate the expression of the Ti-encoded virulence (vir) genes responsible for DNA transfer. Stachel et al., 1986; Rogowsky et al., 1987. Although the basic elements of the phosphorelay between VirA, the transmembrane histidine kinase transmitter, and VirG, the aspartate response regulator, are known, molecular dissection of signal activation is complicated by the absence of a suitable expression system that will overcome higher regulatory controls. For example, virG expression is regulated by environmental factors such as pH and phosphate concentration, factors which also effect the activity of other components of the signal transduction pathway—in particular, VirA activity. Further the native virA promoter is weakly constitutive, and expression of both virA and virG are autocatalytically upregulated in response to plant signals. Stachel et al., 1986; Winans et al., 1988.

Agrobacterium-mediated transformation is most efficient in dicot plants and is the preferable method for transformation of dicots, including Arabidopsis, tobacco, tomato, and potato. Indeed, while Agrobacterium-mediated transformation has been routinely used with dicot plants for a number of years, it has only recently become applicable to monocot plants. Advances in Agrobacterium-mediated transformation techniques have now made the technique applicable to nearly all monocot plants. For example, Agrobacterium-mediated transformation techniques have now been applied to rice (Hiei et al., 1997; Zhang et al., 1997; U.S. Pat. No. 5,591,616, specifically incorporated herein by reference in its entirety), wheat (McCormac et al., 1998), barley (Tingay et al., 1997; McCormac et al., 1998), and maize (Ishidia et al., 1996).

Some Agrobacterium transformation vectors are capable of replication in E. coli as well as Agrobacterium, allowing for convenient manipulations as described. Klee et al., 1985. Moreover, recent technological advances in vectors for Agrobacterium-mediated gene transfer have improved the arrangement of genes and restriction sites in the vectors to facilitate the construction of vectors capable of expressing various polypeptide coding genes. The vectors described have convenient multi-linker regions flanked by a promoter and a polyadenylation site for direct expression of inserted polypeptide coding genes and are suitable for present purposes. Rogers et al., 1987. In addition, Agrobacterium containing both armed and disarmed Ti genes can be used for the transformations. In those plant strains where Agrobacterium-mediated transformation is efficient, it is the method of choice because of the facile and defined nature of the gene transfer.

In a particular embodiment, the present invention provides an expression construct that includes a P_{N25} promoter from coliphage T5, and the genes for virA and virG. The virA gene is a deletion mutant that lacks part of the coding region for the VirA product, namely, an amino-terminal deletion. This construct, when transferred into Agrobacterium, provides for simplified vir gene expression, and can avoid negative regulatory effects of environmental factors such as DIMBOA.

The coliphage T5 promoter (P_{N25}) is one of the strongest transcription initiators in E. coli, and has been shown to be functional in certain gram negative, e.g., Pseudomonas putida and gram positive, e.g., Bacillus subtilis organisms. Gentz et al., 1985. P_{N25} also directs the synthesis of capped or uncapped mRNA in vitro, therefore permitting the expression of genes of eukaryotic origin including wheat germ, reticulocyte and HeLa cells. Bujard et al., 1987. There are relatively few regulatable expression vectors suitable for use in both E. coli and other bacteria, for example, the gram negative plant pathogen Agrobacterium tumefaciens. Jin et al., 1990a, 1990b; Chen et al., 1990; Lohrke et al., 1999; Newman et al., 1999.

The present invention provides a means of expressing the vir proteins in both A. tumefaciens and E. coli such that they can be regulated by signals that do not interact with the VirA/VirG signal transduction machinery. The success of previous attempts to express functional VirA ant VirG in other organisms has been limited. Jin et al, 1990a; 1990b; Chen et al., 1990; Lohrke et al., 1999. The broad-host-range expression system described here successfully drives the expression of a mutant allele of virA, virA (Δ1-284, G665D) and virG in both Agrobacterium and E. coli. Moreover, this greatly simplifies the complex regulation of these virulence proteins in Agrobacterium. This hybrid expression vector is generally useful for the expression of functional proteins in many other organisms.

B. Other Transformation Methods

In other embodiments, alternative method for plant transformation can be used, and are believed to include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., 1985), by electroporation (U.S. Pat. No. 5,384,253, specifically incorporated herein by reference in its entirety), by agitation with silicon carbide fibers (Kaeppler et al., 1990; U.S. Pat. No. 5,302,523, specifically incorporated herein by reference in its entirety; and U.S. Pat. No. 5,464,765, specifically incorporated herein by reference in its entirety), and by acceleration of DNA coated particles U.S. Pat. No. 5,550,318; U.S. Pat. No. 5,538,877; and U.S. Pat. No. 5,538,880; each specifically incorporated herein by reference in its entirety), etc.

Application of these Systems to different plant strains depends upon the ability to regenerate that particular plant strain from protoplasts. Illustrative methods for the regeneration of cereals from protoplasts have been described (Toriyama et al., 1986; Yamada et al., 1986; Abdullah et al., 1986; Omirulleh et al., 1993 and U.S. Pat. No. 5,508,184; each specifically incorporated herein by reference in its entirety). Examples of the use of direct uptake transformation of cereal protoplasts include transformation of rice (Ghosh-Biswas et al., 1994), sorghum (Battraw and Hall, 1991), barley (Lazerri, 1995), oat (Zheng and Edwards, 1990) and maize (Omirulleh et al., 1993).

To transform plant strains that cannot be successfully regenerated from protoplasts, other ways to introduce DNA into intact cells or tissues can be utilized. For example, regeneration of cereals from immature embryos or explants can be effected as described (Vasil, 1989). Also, silicon carbide fiber-mediated transformation may be used with or without protoplasting (Kaeppler, 1990; Kaeppler et al., 1992; U.S. Pat. No. 5,563,055, specifically incorporated herein by reference in its entirety). Transformation with this technique is accomplished by agitating silicon carbide fibers together with cells in a DNA solution. DNA passively enters as the cell are punctured. This technique has been used successfully with, for example, the monocot cereals maize (PCT Application WO 95/06128, specifically incorporated herein by reference in its entirety; Thompson, 1995) and rice (Nagatani, 1997).

V. Method Selecting MDIBOA/DIMBOA Resistant and Phenol Sensitive Mutants

One of the embodiments of the present invention involves selection of Agrobacterium mutants that have improved characteristics for use in plant cell transformation. The present inventors have taken two approaches in achieving this goal. First, the inventors have focused on the inhibitory effects of MDIBOA/DIMBOA on Agrobacterium transformation. Second, the inventors shifted their attention to the inducing effects of phenolics on vir gene expression. Following both these paths, the inventors have developed new strains of Agrobacterium that are able to transform cells more efficiently that parental strains.

A. MDIBOA/DIMBOA Resistant Mutants

A. tumefaciens strain A348/pAC2, which contains the kanamycin resistance gene under the control of the virB promoter. At 40 μg/ml kanamycin, these cells survive with 100 μM AS induction, but die at 30 μM AS, demonstrating the phenolic induction of kanamycin resistance is provided by pAC2 (vector controls died at all AS concentrations). This observation permitted.

DIMBOA resistant mutants are selected by growth in DIMBOA, where variants resistant to the inhibitory effects of DIMBOA (i.e., vir gene expression still takes place) are able to proliferate. In a more specific embodiment, the inhibition of vir genes is assessed by linking a vir promoter to an antibiotic resistance gene. As described in the examples, individual colonies of A348/pAC2 are grown overnight in LB broth plus spectinomycin, and the progeny from each colony kept separate to ensure that each mutation is an individual event, not part of a clonal population. The bacteria are induced overnight in media plus AS. The next day, the cells are plated onto plates containing medium plus AS, DIMBOA plus kanamycin. Colonies that grow on this selection medium are subsequently assayed for growth on plates containing kanamycin but no phenolic. Colonies that grow under these conditions are constitutive for kanamycin resistance and are not characterized further, while colonies that do not survive in this secondary screen are characterized further. An exemplary mutant that is resistant to DIMBOA is designated JZ03 (ATCC #______, Deposited Nov. 5, 1999).

Other suitable antiobiotic selections that may be employed include chloramphenicol, tetracycline, and ampicillin.

B. Phenol Hypersensitive Mutants

In a method similar to that described above, mutants of Agrobacterium are selected that have an increased sensitivity to phenolic induction. The principle behind the screen is by lowering the threshold phenolic level at which vir genes are activated, one will also increase the vir gene response in the wild, helping to overcome natural barriers to vir gene expression.

Phenolic hypersensitive mutants are selected as follows. Individual colonies of A348/pAC2, where the kanamycin gene is placed under the control of the virB promoter, are grown overnight in LB broth plus spectinomycin, and the progeny from each colony kept separate to ensure that each mutation is an individual event, not part of a clonal population. The bacteria are subsequently washed, resuspended and induced overnight in AS. The next day, bacteria are plated onto plates containing AS and kanamycin. Colonies that grow on this selection medium are subsequently assayed for growth on plates containing kanamycin but no phenolic. Colonies that grow under these conditions are constitutive for kanamycin resistance and are not characterized further, while colonies that do not survive in this screen are characterized further.

The kanamycin resistance test of selected colonies in response to different doses of phenol is performed as follows: after one night of induction in ABIM over a range of inducer concentrations, as described above, the bacteria are washed and subcultured. The phenolics range from of concentrations of 0.1 to 1000 μM. At each concentration of inducer three cultures have kanamycin at a concentration of 20 μg/mL, while one culture has no kanamycin. After overnight culture the OD₆₀₀ is determined and kanamycin resistant growth as a percentage of control is determined using the following calculation:

$$\% growth = \frac{( {OD}_{600} overnight culture + kanamycin ) \times 100}{( {OD}_{600} overnight culture w / no kanamnycin )}$$

The mean of three samples is then plotted versus log of concentration of inducer.

VI. Recipient Cells for Transformation

Tissue culture requires media and controlled environments. “Media” refers to the numerous nutrient mixtures that are used to grow cells in vitro, that is, outside of the intact living organism. The medium usually is a suspension of various categories of ingredients (salts, amino acids, growth regulators, sugars, buffers) that are required for growth of most cell types. However, each specific cell type requires a specific range of ingredient proportions for growth, and an even more specific range of formulas for optimum growth. Rate of cell growth also will vary among cultures initiated with the array of media that permit growth of that cell type.

Nutrient media is prepared as a liquid, but this may be solidified by adding the liquid to materials capable of providing a solid support. Agar is most commonly used for this purpose. Bactoagar, Hazelton agar, Gelrite, and Gelgro are specific types of solid support that are suitable for growth of plant cells in tissue culture.

Some cell types will grow and divide either in liquid suspension or on solid media. As disclosed herein, plant cells will grow in suspension or on solid medium, but regeneration of plants from suspension cultures typically requires transfer from liquid to solid media at some point in development. The type and extent of differentiation of cells in culture will be affected not only by the type of media used and by the environment, for example, pH, but also by whether media is solid or liquid.

Recipient cell targets include, but are not limited to, meristem cells, Type I, Type II, and Type III callus, immature embryos and gametic cells such as microspores, pollen, sperm and egg cells. It is contemplated that any cell from which a fertile plant may be regenerated is useful as a recipient cell. Type I, Type II, and Type III callus may be initiated from tissue sources including, but not limited to, immature embryos, seedling apical meristems, microspores and the like. Those cells which are capable of proliferating as callus also are recipient cells for genetic transformation. The present invention provides techniques for transforming immature embryos and subsequent regeneration of fertile transgenic plants. Transformation of immature embryos obviates the need for long term development of recipient cell cultures. Pollen, as well as its precursor cells, microspores, may be capable of functioning as recipient cells for genetic transformation, or as vectors to carry foreign DNA for incorporation during fertilization Direct pollen transformation would obviate the need for cell culture. Meristematic cells (i.e., plant cells capable of continual cell division and characterized by an undifferentiated cytological appearance, normally found at growing points or tissues in plants such as root tips, stem apices, lateral buds, etc.) may represent another type of recipient plant cell. Because of their undifferentiated growth and capacity for organ differentiation and totipotency, a single transformed meristematic cell could be recovered as a whole transformed plant. In fact, it is proposed that embryogenic suspension cultures may be an in vitro meristematic cell system, retaining an ability for continued cell division in an undifferentiated state, controlled by the media environment.

Cultured plant cells that can serve as recipient cells for transforming with desired DNA segments may be any plant cells including corn cells, and more specifically, cells from Zea mays L. Somatic cells are of various types. Embryogenic cells are one example of somatic cells which may be induced to regenerate a plant through embryo formation. Non-embryogenic cells are those which typically will not respond in such a fashion. An example of non-embryogenic cells are certain Black Mexican Sweet (BMS) corn cells.

The development of embryogenic maize calli and suspension cultures useful in the context of the present invention, e.g., as recipient cells for transformation, has been described in U.S. Pat. No. 5,134,074; and U.S. Pat. No. 5,489,520; each of which is incorporated herein by reference in its entirety.

Certain techniques may be used that enrich recipient cells within a cell population. For example, Type II callus development, followed by manual selection and culture of friable, embryogenic tissue, generally results in an enrichment of recipient cells for use in transformation. Suspension culturing, particularly using the media disclosed herein, may improve the ratio of recipient to non-recipient cells in any given population. Manual selection techniques which can be employed to select recipient cells may include, e.g., assessing cell morphology and differentiation, or may use various physical or biological means. Cryopreservation also is a possible method of selecting for recipient cells.

Manual selection of recipient cells, e.g., by selecting embryogenic cells from the surface of a Type II callus, is one means that may be used in an attempt to enrich for recipient cells prior to culturing (whether cultured on solid media or in suspension). The preferred cells may be those located at the surface of a cell cluster, and may further be identifiable by their lack of differentiation, their size and dense cytoplasm. The preferred cells will generally be those cells which are less differentiated, or not yet committed to differentiation. Thus, one may wish to identify and select those cells which are cytoplasmically dense, relatively unvacuolated with a high nucleus to cytoplasm ratio (e.g., determined by cytological observations), small in size (e.g., 10-20 μm), and capable of sustained divisions and somatic proembryo formation.

It is proposed that other means for identifying such cells also may be employed. For example, through the use of dyes, such as Evan's blue, which are excluded by cells with relatively non-permeable membranes, such as embryogenic cells, and taken up by relatively differentiated cells such as root-like cells and snake cells (so-called due to their snake-like appearance).

Other possible means of identifying recipient cells include the use of isozyme markers of embryogenic cells, such as glutamate dehydrogenase, which can be detected by cytochemical stains (Fransz et al., 1989). However, it is cautioned that the use of isozyme markers including glutamate dehydrogenase may lead to some degree of false positives from non-embryogenic cells such as rooty cells which nonetheless have a relatively high metabolic activity.

A. Culturing Cells to be Recipients for Transformation

The ability to prepare and cryopreserve cultures of plant cells is important to certain aspects of the present invention, in that it provides a means for reproducibly and successfully preparing cells for transformation. A variety of different types of media have been previously developed and may be employed in carrying out various aspects of the invention.

B. Media

In certain embodiments of the current invention, recipient cells may be selected following growth in culture. Where employed, cultured cells may be grown either on solid supports or in the form of liquid suspensions. In either instance, nutrients may be provided to the cells in the form of media, and environmental conditions controlled. There are many types of tissue culture media comprised of various amino acids, salts, sugars, growth regulators and vitamins. Most of the media employed in the practice of the invention will have some similar components, but may differ in the composition and proportions of their ingredients depending on the particular application envisioned. For example, various cell types usually grow in more than one type of media, but will exhibit different growth rates and different morphologies, depending on the growth media. In some media, cells survive but do not divide.

Various types of media suitable for culture of plant cells previously have been described. Examples of these media include, but are not limited to, the N6 medium described by Chu et al. is (1975) and MS media (Murashige and Skoog, 1962). It has been discovered that media such as MS which have a high ammonia/nitrate ratio are counterproductive to the generation of recipient cells in that they promote loss of morphogenic capacity. N6 media, on the other hand, has a somewhat lower ammonia/nitrate ratio, and is contemplated to promote the generation of recipient cells by maintaining cells in a proembryonic state capable of sustained divisions.

C. Maintenance

The method of maintenance of cell cultures may contribute to their utility as sources of recipient cells for transformation. Manual selection of cells for transfer to fresh culture medium, frequency of transfer to fresh culture medium, composition of culture medium, and environmental factors including, but not limited to, light quality and quantity and temperature are all important factors in maintaining callus and/or suspension cultures that are useful as sources of recipient cells. It is contemplated that alternating callus between different culture conditions may be beneficial in enriching for recipient cells within a culture. For example, it is proposed that cells may be cultured in suspension culture, but transferred to solid medium at regular intervals. After a period of growth on solid medium cells can be manually selected for return to liquid culture medium. It is proposed that by repeating this sequence of transfers to fresh culture medium it is possible to enrich for recipient cells. It also is contemplated that passing cell cultures through a 1.9 mm sieve is useful in maintaining the friability of a callus or suspension culture and may be beneficial in enriching for transformable cells.

D. Cryopreservation Methods

Cryopreservation is important because it allows one to maintain and preserve a known transformable cell culture for future use, while eliminating the cumulative detrimental effects associated with extended culture periods. Cell suspensions and callus were cryopreserved using modifications of methods previously reported (Finkle, 1985; Withers & King, 1979). The cryopreservation protocol comprised adding a pre-cooled (0° C.) concentrated cryoprotectant mixture stepwise over a period of one to two hours to precooled (0° C.) cells. The mixture was maintained at 0° C. throughout this period. The volume of added cryoprotectant was equal to the initial volume of the cell suspension (1:1 addition), and the final concentration of cryoprotectant additives was 10% dimethyl sulfoxide, 10% polyethylene glycol (6000 MW), 0.23 M proline and 0.23 M glucose. The mixture was allowed to equilibrate at 0° C. for 30 minutes, during which time the cell suspension/cryoprotectant mixture was divided into 1.5 ml aliquot (0.5 ml packed cell volume) in 2 ml polyethylene cryo-vials. The tubes were cooled at 0.5° C./minute to −8° C. and held at this temperature for ice nucleation.

Once extracellular ice formation had been visually confirmed, the tubes were cooled at 0.5° C./minute from −8° C. to −35° C. They were held at this temperature for 45 minutes (to insure uniform freeze-induced dehydration throughout the cell clusters). At this point, the cells had lost the majority of their osmotic volume (i.e., there is little free water left in the cells), and they could be safely plunged into liquid nitrogen for storage. The paucity of free water remaining in the cells in conjunction with the rapid cooling rates from −35° C. to −196° C. prevented large organized ice crystals from forming in the cells. The cells are stored in liquid nitrogen, which effectively immobilizes the cells and slows metabolic processes to the point where long-term storage should not be detrimental.

Thawing of the extracellular solution was accomplished by removing the cryo-tube from liquid nitrogen and swirling it in sterile 42° C. water for approximately 2 minutes. The tube was removed from the heat immediately after the last ice crystals had melted to prevent heating the tissue. The cell suspension (still in the cryoprotectant mixture) was pipetted onto a filter, resting on a layer of BMS cells (the feeder layer which provided a nurse effect during recovery). The cryoprotectant solution is removed by pipetting. Culture medium comprised a callus proliferation medium with increased osmotic strength. Dilution of the cryoprotectant occurred slowly as the solutes diffused away through the filter and nutrients diffused upward to the recovering cells. Once subsequent growth of the thawed cells was noted, the growing tissue was transferred to fresh culture medium. If initiation of a suspension culture was desired, the cell clusters were transferred back into liquid suspension medium as soon as sufficient cell mass had been regained (usually within 1 to 2 weeks). Alternatively, cells were cultured on solid callus proliferation medium. After the culture was reestablished in liquid (within 1 to 2 additional weeks), it was used for transformation experiments. When desired, previously cryopreserved cultures may be frozen again for storage.

VII. Production and Characterization of Stably Transformed Plants

After effecting delivery of exogenous DNA to recipient cells, the next steps generally concern identifying the transformed cells for further culturing and plant regeneration. As mentioned herein, in order to improve the ability to identify transformants, one may desire to employ a selectable or screenable marker gene as, or in addition to, the expressible gene of interest. In this case, one would then generally assay the potentially transformed cell population by exposing the cells to a selective agent or agents, or one would screen the cells for the desired marker gene trait.

A. Selection

It is believed that DNA is introduced into only a small percentage of target cells in any one experiment. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Resistance to the aminoglycoside antibiotics is conferred by aminoglycoside phosphostransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA. Using the techniques disclosed herein, greater than 40% of bombarded embryos may yield transformants.

One herbicide which constitutes a desirable selection agent is the broad spectrum herbicide bialaphos. Bialaphos is a tripeptide antibiotic produced by Streptomyces hygroscopicus and is composed of phosphinothricin (PPT), an analogue of L-glutamic acid, and two L-alanine residues. Upon removal of the L-alanine residues by intracellular peptidases, the PPT is released and is a potent inhibitor of glutamine synthetase (GS), a pivotal enzyme involved in ammonia assimilation and nitrogen metabolism (Ogawa et al., 1973). Synthetic PPT, the active ingredient in the herbicide Liberty™ also is effective as a selection agent. Inhibition of GS in plants by PPT causes the rapid accumulation of ammonia and death of the plant cells.

The organism producing bialaphos and other species of the genus Streptomyces also synthesizes an enzyme phosphinothricin acetyl transferase (PAT) which is encoded by the bar gene in Streptomyces hygroscopicus and the pat gene in Streptomyces viridochromogenes. The use of the herbicide resistance gene encoding phosphinothricin acetyl transferase (PAT) is referred to in DE 3642 829 A, wherein the gene is isolated from Streptomyces viridochromogenes. In the bacterial source organism, this enzyme acetylates the free amino group of PPT preventing auto-toxicity (Thompson et al., 1987). The bar gene has been cloned (Murakami et al., 1986; Thompson et al., 1987) and expressed in transgenic tobacco, tomato, potato (De Block et al., 1987) Brassica (De Block et al., 1989) and maize U.S. Pat. No. 5,550,318). In previous reports, some transgenic plants which expressed the resistance gene were completely resistant to commercial formulations of PPT and bialaphos in greenhouses.

Another example of a herbicide which is useful for selection of transformed cell lines in the practice of the invention is the broad spectrum herbicide glyphosate. Glyphosate inhibits the action of the enzyme EPSPS which is active in the aromatic amino acid biosynthetic pathway. Inhibition of this enzyme leads to starvation for the amino acids phenylalanine, tyrosine, and tryptophan and secondary metabolites derived thereof. U.S. Pat. No. 4,535,060 describes the isolation of EPSPS mutations which confer glyphosate resistance on the Salmonella typhimurium gene for EPSPS, aroA. The EPSPS gene was cloned from Zea mays and mutations similar to those found in a glyphosate resistant aroA gene were introduced in vitro. Mutant genes encoding glyphosate resistant EPSPS enzymes are described in, for example, International Patent WO 97/4103. The best characterized mutant EPSPS gene conferring glyphosate resistance comprises amino acid changes at residues 102 and 106, although it is anticipated that other mutations also will be useful (PCT/WO97/4103).

To use the bar-bialaphos or the EPSPS-glyphosate selective system, bombarded tissue is cultured for 0-28 days on nonselective medium and subsequently transferred to medium containing from 1-3 mg/l bialaphos or 1-3 mM glyphosate as appropriate. While ranges of 1-3 mg/l bialaphos or 1-3 mM glyphosate will typically be preferred, it is proposed that ranges of 0.1-50 mg/l bialaphos or 0.1-50 mM glyphosate will find utility in the practice of the invention. Tissue can be placed on any porous, inert, solid or semi-solid support for bombardment, including but not limited to filters and solid culture medium. Bialaphos and glyphosate are provided as examples of agents suitable for selection of transformants, but the technique of this invention is not limited to them.

It further is contemplated that the herbicide DALAPON, 2,2-dichloropropionic acid, may be useful for identification of transformed cells. The enzyme 2,2-dichloropropionic acid dehalogenase (deh) inactivates the herbicidal activity of 2,2-dichloropropionic acid and therefore confers herbicidal resistance on cells or plants expressing a gene encoding the dehalogenase enzyme (Buchanan-Wollaston et al., 1992; U.S. Pat. No. 5,508,468; and U.S. Pat. No. 5,508,468; each of the disclosures of which is specifically incorporated herein by reference in its entirety).

Alternatively, a gene encoding anthranilate synthase, which confers resistance to certain amino acid analogs, e.g., 5-methyltryptophan or 6-methyl anthranilate, may be useful as a selectable marker gene. The use of an anthranilate synthase gene as a selectable marker was described in U.S. Pat. No. 5,508,468.

An example of a screenable marker trait is the red pigment produced under the control of the R-locus in maize. This pigment may be detected by culturing cells on a solid support containing nutrient media capable of supporting growth at this stage and selecting cells from colonies (visible aggregates of cells) that are pigmented. These cells may be cultured further, either in suspension or on solid media. The R-locus is useful for selection of transformants from bombarded immature embryos. In a similar fashion, the introduction of the C1 and B genes will result in pigmented cells and/or tissues.

The enzyme luciferase may be used as a screenable marker in the context of the present invention. In the presence of the substrate luciferin, cells expressing luciferase emit light which can be detected on photographic or x-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells which are expressing luciferase and manipulate those in real time. Another screenable marker which may be used in a similar fashion is the gene coding for green fluorescent protein.

It further is contemplated that combinations of screenable and selectable markers will be useful for identification of transformed cells. In some cell or tissue types a selection agent, such as bialaphos or glyphosate, may either not provide enough killing activity to clearly recognize transformed cells or may cause substantial nonselective inhibition of transformants and nontransformants alike, thus causing the selection technique to not be effective. It is proposed that selection with a growth inhibiting compound, such as bialaphos or glyphosate at concentrations below those that cause 100% inhibition followed by screening of growing tissue for expression of a screenable marker gene such as luciferase would allow one to recover transformants from cell or tissue types that are not amenable to selection alone. It is proposed that combinations of selection and screening may enable one to identify transformants in a wider variety of cell and tissue types. This may be efficiently achieved using a gene fusion between a selectable marker gene and a screenable marker gene, for example, between an NPTII gene and a GFP gene.

B. Regeneration and Seed Production

Cells that survive the exposure to the selective agent, or cells that have been scored positive in a screening assay, may be cultured in media that supports regeneration of plants. In an exemplary embodiment, MS and N6 media may be modified by including further substances such as growth regulators. A preferred growth regulator for such purposes is dicamba or 2,4-D. However, other growth regulators may be employed, including NAA, NAA+2,4-D or perhaps even picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 wk, then transferred to media conducive to maturation of embryoids. Cultures are transferred every 2 wk on this medium. Shoot development will signal the time to transfer to medium lacking growth regulators.

The transformed cells, identified by selection or screening and cultured in an appropriate medium that supports regeneration, will then be allowed to mature into plants. Developing plantlets are transferred to soiless plant growth mix, and hardened, e.g., in an environmentally controlled chamber at about 85% relative humidity, 600 ppm CO₂, and 25-250 microeinsteins m⁻²s⁻¹ of light. Plants are preferably matured either in a growth chamber or greenhouse. Plants are regenerated from about 6 wk to 10 months after a transformant is identified, depending on the initial tissue. During regeneration, cells are grown on solid media in tissue culture vessels. Illustrative embodiments of such vessels are petri dishes and Plant Cons. Regenerating plants are preferably grown at about 19 to 28° C. After the regenerating plants have reached the stage of shoot and root development, they may be transferred to a greenhouse for further growth and testing.

Note, however, that seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/l agarose. In embryo rescue, large embryos (defined as greater than 3 mm in length) are germinated directly on an appropriate media. Embryos smaller than that may be cultured for 1 wk on media containing the above ingredients along with 10⁻⁵ M abscisic acid and then transferred to growth regulator-free medium for germination.

Progeny may be recovered from transformed plants and tested for expression of the exogenous expressible gene by localized application of an appropriate substrate to plant parts such as leaves. In the case of bar transformed plants, it was found that transformed parental plants (R_{O}) and their progeny of any generation tested exhibited no bialaphos-related necrosis after localized application of the herbicide Basta to leaves, if there was functional PAT activity in the plants as assessed by an in vitro enzymatic assay. All PAT positive progeny tested contained bar, confirming that the presence of the enzyme and the resistance to bialaphos were associated with the transmission through the germline of the marker gene.

C. Characterization

To confirm the presence of the exogenous DNA or “transgene(s)” in the regenerating plants, a variety of assays may be performed. Such assays include, for example, “molecular biological” assays, such as Southern and Northern blotting and PCR™; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

1. DNA Integration, RNA Expression and Inheritance

Genomic DNA may be isolated from callus cell lines or any plant parts to determine the presence of the exogenous gene through the use of techniques well known to those skilled in the art. Note, that intact sequences will not always be present, presumably due to rearrangement or deletion of sequences in the cell.

The presence of DNA elements introduced through the methods of this invention may be determined by polymerase chain reaction (PCR™). Using this technique discreet fragments of DNA are amplified and detected by gel electrophoresis. This type of analysis permits one to determine whether a gene is present in a stable transformant, but does not prove integration of the introduced gene into the host cell genome. It is the experience of the inventor, however, that DNA has been integrated into the genome of all transformants that demonstrate the presence of the gene through PCR™ analysis. In addition, it is not possible using PCR™ techniques to determine whether transformants have exogenous genes introduced into different sites in the genome, i.e., whether transformants are of independent origin. It is contemplated that using PCR™ techniques it would be possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR™, e.g., the presence of a gene, but also demonstrates integration into the genome and characterizes each individual transformant.

It is contemplated that using the techniques of dot or slot blot hybridization which are modifications of Southern hybridization techniques one could obtain the same information that is derived from PCR™, e.g., the presence of a gene.

Both PCR™ and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR™ techniques also may be used for detection and quantitation of RNA produced from introduced genes. In this application of PCR™ it is first necessary to reverse transcribe RNA into DNA, using enzymes such as reverse transcriptase, and then through the use of conventional PCR™ techniques amplify the DNA. In most instances PCR™ techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species also can be determined using dot or slot blot Northern hybridizations. These techniques are modifications of Northern blotting and will only demonstrate the presence or absence of an RNA species.

2. Gene Expression

While Southern blotting and PCR™ may be used to detect the gene(s) in question, it does not provide information as to whether the gene is being expressed. Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and ¹⁴C-acetyl CoA or for anthranilate synthase activity by following loss of fluorescence of anthranilate, to name two.

Very frequently the expression of a gene product is determined by evaluating the phenotypic results of its expression. These assays also may take many forms including but not limited to analyzing changes in the chemical composition, morphology, or physiological properties of the plant. Chemical composition may be altered by expression of genes encoding enzymes or storage proteins that change amino acid composition and may be detected by amino acid analysis, or by enzymes which change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.

VIII. Breeding Plants of the Invention

In addition to direct transformation of a particular genotype with a construct prepared according to the current invention, transgenic plants may be made by crossing a plant having a construct of the invention to a second plant lacking the construct. For example, a selected gene can be introduced into a particular plant variety by crossing, without the need for ever directly transforming a plant of that given variety. Therefore, the current invention not only encompasses a plant directly regenerated from cells that have been transformed in accordance with the current invention, but also the progeny of such plants. As used herein the term “progeny” denotes the offspring of any generation of a parent plant prepared in accordance with the instant invention, wherein the progeny comprises a construct prepared in accordance with the invention. “Crossing” a plant to provide a plant line having one or more added transgenes relative to a starting plant line, as disclosed herein, is defined as the techniques that result in a transgene of the invention being introduced into a plant line by crossing a starting line with a donor plant line that comprises a transgene of the invention. To achieve this one could, for example, perform the following steps:

(a) plant seeds of the first (starting line) and second (donor plant line that comprises a transgene of the invention) parent plants;

(b) grow the seeds of the first and second parent plants into plants that bear flowers;

(c) pollinate a flower (female flower) of the first parent plant with the pollen of the second parent plant; and

(d) harvest seeds produced on the parent plant bearing the female flower.

Backcrossing is herein defined as the process including the steps of:

(a) crossing a plant of a first genotype containing a desired gene, DNA sequence or element to a plant of a second genotype lacking said desired gene, DNA sequence or element;

(b) selecting one or more progeny plant containing the desired gene, DNA sequence or element;

(c) crossing the progeny plant to a plant of the second genotype; and

(d) repeating steps (b) and (c) for the purpose of transferring said desired gene, DNA sequence or element from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking said desired DNA sequence may be referred to as an unconverted genotype, line, inbred, or hybrid.

IX. Definitions

Genetic Transformation: A process of introducing a DNA sequence or construct (e.g., a vector or expression cassette) into a cell or protoplast in which that exogenous DNA is incorporated into a chromosome or is capable of autonomous replication.

Enhancer: A genetic element that is capable of enhancing the expression of a heterologous gene.

Expression: The combination of intracellular processes, including transcription and translation undergone by a coding DNA molecule such as a structural gene to produce a

Expression cassette: A segment of DNA which has been isolated from a vector. An exemplary expression cassette comprises a segment of DNA excised from a plasmid vector, wherein bacterial sequences such as an origin of replication or bacterial selectable marker have been partially or completely deleted from the expression cassette.

Expression vector: A chimeric DNA molecule which is designed for introduction into a host genome by genetic transformation. Preferred expression vectors will comprise all of the genetic elements necessary to direct the expression of one or more selected genes. In particular embodiments of the invention, it may be desirable to introduce an expression vector into a host cell in the form an expression cassette.

Heterologous Gene: A translated nucleic acid sequence which is operably linked to a terminator other than the terminator to which the corresponding nucleic acid sequence is operably linked in naturally occurring, non-recombinant cells from which the nucleic acid was originally isolated or derived.

Progeny: Any subsequent generation, including the seeds and plants therefrom, which is derived from a particular parental plant or set of parental plants.

Promoter: A recognition site on a DNA sequence or group of DNA sequences that provide an expression control element for a structural gene and to which RNA polymerase specifically binds and initiates RNA synthesis (transcription) of that gene.

R₀ Transgenic Plant: A plant which has been directly transformed with a selected DNA or has been regenerated from a cell or cell cluster which has been transformed with a selected DNA.

Regeneration: The process of growing a plant from a plant cell (e.g., plant protoplast or explant).

Selected DNA: A DNA segment which was stably introduced into a plant genome by genetic transformation.

Selected Gene: A sequence which one desires to have expressed in a transgenic plant, plant cell or plant part. A selected gene may be native or foreign to a host genome, but where the selected gene is present in the host genome, will include one or more regulatory or functional elements which alter the expression profile of the selected gene relative to native copies of the gene.

Transformed cell: A cell the DNA complement of which has been altered by the introduction of an exogenous DNA molecule into that cell.

Transgene: A segment of DNA which has been incorporated into a host genome or is capable of autonomous replication in a host cell and is capable of causing the expression of one or more cellular products. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation that was transformed with the transgene.

Transgenic plant: A plant or progeny of any subsequent generation derived therefrom, wherein the DNA of the plant or progeny thereof contains an introduced exogenous DNA molecule not originally present in a non-transgenic plant of the same strain. The transgenic plant may additionally contain sequences that are native to the plant being transformed, but wherein the “exogenous” gene has been altered by gene technological means in order to alter the level or pattern of expression of the gene.

Transit peptide: A polypeptide sequence which is capable of directing a polypeptide to a particular organelle or other location within a cell.

### X. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

Identification of a Maize Resistance Factor

A. Materials and Methods

Proton (¹H) NMR spectra and Carbon (¹³C) NMR spectra were either obtained on the U. of Chicago Bruker Avance/DRX500 and Bruker Avance/DRX400 spectrometer that was equipped with a Nicolet 1280 data acquisition system operated at 500 MHz or 400 MHz. Low resolution mass spectra (MS) were obtained using a UG 70-250 Mass spectrometer. High pressure liquid chromatography (HPLC) system was a Rainin system which included Model HPXL pumps pressure module max; 8700 psi), dynamax Absorbance detector (model UV-D) and a Macintegrator software installed into the MacPlus computer. Ultraviolet (UV) spectra were recorded with a Perkin-Elmer lambda 6 UV/VIS spectrophotometer in quartz curvettes with an optical pathway of 1 cm. Circular dichroism (CD) spectroscopy was measured using a Jasco-600 CD spectrophotometer. Optical rotation was measured with a Perkin-Elmer 141 polarimeter. Ozonolysis is carried out with a Welsbach model T-408 laboratory ozonicr, Welsbach Corp., Philadelphia, Pa.

Seeds, reagents and methods Sterlization was by treatment in a Market Forge Sterilmatic at 15 lb/in² and 121° C. for 25 mins.

Flats Seed Corn (100 ml) was surface sterilized with 30% commercial bleach (20 min), washed with ddH₂O, and planted in an autoclavable box (12″×24″×6″) containing sterile moist vermiculate for 7-11 days. The roots of the seedlings were dipped in either CH₂Cl₂ containing 0.5% HOAc or EtOAc for 3 seconds, and the solvent was concentrated in vacuo, and analyzed by HPLC (Microsorb SiO₂, cyclohexane/ethyl acetate (67:33 v/v)) on a Rainin MPXL dual-pump with detection at 254 nm.

Syntheses. Acetosyringone (AS) was purchased from Aldrich and used directly. HPLC grade solvents were purchased from Fisher Chemical Co. or J.T. Baker Chemical Co. Tetrahydrofuran (THF) and diethyl ether were distilled from sodium/benzophenone and dichloromethane (CH₂Cl₂) was distilled from calcium hydride. H₂0 was double distilled from all glass stilts.

Flash column chromatography employed EM Science silica gel, 230-400 mesh, 40-63 Å in diameter. Thin layer chromatography was performed with Baker-flex silica gel IB2F plastic backed plates with a 0.25 mm silica gel layer. Plates were visualized by 254 nm illumination, and/or treatment with Aldrich phosphomolybdic acid reagent with 2-fold dilution with absolute ethanol.

2-hydroxy4,7-dimethoxybenzoxazin-3-one (1). Following a previous procedure, 10-day-old corn seedlings (200 g) were frozen in N₂ (1) and homogenized in a Waring blender in 400 ml of water, agitated at room temperature for 2 hrs to allow for enzymatic hydrolysis of DIMBOA glucoside and clarified twice at 4,000 rpm for 15 mm in a Sorval RC5S with a GSA rotor or simply filtered through Celite twice. Sahi et al., 1991. The brown aqueous layer was extracted three times with an equal volume of EtOAc, and the organic layer was dried over MgSO₄, concentrated and purified over SiO₂. The product gave a positive FeCI₃ test and ¹H-NMR (400 MHz, C₃D₆O) δ7.25 (d, 1H, J=8.8 Hz, C5-H), 6.7 (dd, 1H, J=2.6, 8.8 Hz, C6-H), 6.6 (d, IH, J=2.6 Hz, C8-H), 5.7 (s, IH, C2-H), 3.8 (s, 3H, OCH₃).

DIMBOA (0.05 mmol) was dissolved in 5 ml of anhydrous diethyl ether and treated with an excess of alcohol free diazomethane ethereal solution (0.33 mmol) at 0° C. Alcohol-free dizomethane prepared in situ in diethyl ether limited nonselective methylation of the phenol ring-chain tautomer. The reaction was monitored by the disappearance of both the yellow CH₂N₂ and the positive FeCl₃ test. The crude mixture was concentrated in vacuo and rapidly chromatographed (SsO₂, hexane/ethyl acetate, 1:1) to afford I (40%). ¹H NMR(500 MHz, NMR(500 MHz, CDCl₃) 157.5, 156.9, 141.9, 119.7, 114.0, 108.5, 104.0, 92.3, 62.8, 55.7. MS (CI) m/Z (relative intensity) 226 (10, M+H), 208(10). 194(20), 180(10), 166(100); HRMS (EI) calculated for C₁₀H₁₁NO₅. IR 1684.0, 1506.1.

Bacterial strains and Media. The strains and plasmids used are listed below. A. tumefaciens strains: A348 (Garfinkel et al., 1981) has the C58 chromosomal background with pTiA6; 358mx (Stachel & Nester, 1986) is an A348 derivative harboring insertion of the transposon Tn3-HoHol in the virE2 gene of pTiA6 creating a virE::lac Z fusion. Strains JZ101-104 are DIMBOA resistant mutants, derived from strain A348 as described below.

The A. tumefaciens strain A348-3 has virA deleted on the Ti plasmid (Lee et al., 1992), and for this study carries both pMutA (McLean et al., 1994), a single copy plasmid carrying virA with constitutive mutation, G665D, within the kinase domain, and pSW209Ω (Chang & Winans, 1992), a. spec^{R}, IncP derivative of pSW209 with a virB::lacZ reporter construct.

Strains were maintained on LB plates or AB minimal medium (Winans et al., 1988) supplemented with antibiotics as appropriate. AB induction medium (AB1M), pH 5.5 and containing 1% glucose or 1% glycerol ±0.1% arabinose, was used for vir gene expression studies.

TABLE 7

| Strains/plasmids | | |
|---|---|---|
| Strains | Relevant genotype | Source/reference |
| A136 | Strain C58 cured of pTi plasmid | Sciaky et al., 1978 |
| A348 | A136 containing pTiA6 | Garfinkel et al., 1981 |
| 358 mx | An A348 derivative harboring | Stachel & Nester 1986 |
| | insertion of the transposon Tn3- | |
| | HoHo1 in the virE2 gene of pTiA6 | |
| | creating a virE::lac Z fusion | |
| A348-3 | VirA deletion derivative of A348, Km^{r} | Lee et al., 1992 |
| JZ101-104 Plasmids | 4.5 kb Kpn I fragment containing | McClean et al., 1994 |
| pMutA G665D | virA (G665D) in pUCD2 | |
| PAB213-22 | 4.5 kb Kpn I fragment containing | This study |
| | wild-type virA in pUCD2 | |
| PSW209 | IncP, virB::lacZ fusion derived from | S.C. Williams, Cornell |
| | pSW243cd, Km^{r} | University |
| PSW209Ω | 2 kb Bam H1 fragment from pHP45 | Wang et al., 1999 |
| | coloned into Bam HI site of pSW209; | |
| | IneP Spec^{r}, Km^{r} virB::lacZ | |
| PSM102 | IncP, occ::lacZ, Carb^{r} | Stachel et al., 1985 |
| PAC2 | IncP, virB::Pst II, Spec^{r} | Campbell et al., 1999 |

Vir Induction Assay. A. tumefaciens strains were grown overnight at 27° C. to an OD of 0.3-0.6 in LB medium supplemented with appropriate antibiotics. Cells were then pelleted by centrifugation and resuspended to an OD of 0.1 per ml in sterilized induction medium (ABIM, pH 5.5) supplemented with either 1% glucose or 1% glycerol. The various inducer and Inhibitor compounds tested were dissolved in a minimal amount of DMSO in water as mM stocks, and diluted with ABIM to the desired concentration (final DMSO concentration was no greater than 0.1%). The bacteria were incubated with at 28° C. for 8-16 hr and subsequently assayed for β-galactosidase activity by the method of Miller (1986). IC₅₀ values for the Inhibitors evaluated were estimated from such dose response plots. Each assay value reported is the mean of three replicates; error bar indicates one standard deviation from the mean. Data are representative of three similar independent studies.

Plant sensitivity assays. A. tumefaciens strains were grown to mid-logarithmic phase in LB media and cultured as described. Strains were then serially diluted in induction broth; 4 ml volumes of these mixtures (about 10⁶ CFU/ml) were added to 100 ml of ABIM top agar (Induction medium containing 0.7% agar) and overlaid on ABIM plates containing AS. The 5-day-old young seedling of maize was placed on the top agar. The plates were incubated right-side-up at room temperature for 3-5 days. Photographs were taken on the third day for A348/psw209 and on the fifth day for A348/pAC2.

TABLE 8

| Strain | ABIM plate | ABIM top agar |
|---|---|---|
| A348/pSW209 | AS 10 μM | Bacterial suspension (ca. |
| | Xgal 80 μg/ml | 10⁶ CFU/ml) |
| | Kan 50 μg/ml | Kan 50 μg/ml |
| A348/pAC2 | AS 100 μM | Bacterial suspension (ca. |
| | Calcofluor 0.2 μg/ml | 10⁶ CFU/ml) |
| | Kan 50 μg/ml | Calcofluor 1 μg/ml |
| | | Kan 50 μg/ml |

Mutant Selection. Preliminary experiments indicated that A. tumefaciens strain A348/pAC2, grown on induction plates plus 40 μg/ml kanamycin survive at 100 μM AS but are killed at 30 μM AS, demonstrating the phenolic induction of kanamycin resistance is provided by pAC2 (vector controls died at all AS concentrations). DIMBOA resistant mutants were selected as follows. Individual colonies of A348/pAC2 were grown overnight in LB broth plus spectinomycin, and the progeny from each colony kept separate to ensure that each mutation was an individual event, not part of a clonal population. The bacteria were subsequently washed in AB medium, resuspended to an OD₆₀₀ of approximately 0.1 and induced overnight in AB media plus 100 μM AS. The next day the cells had grown to an approximate OD₆₀₀ of 1.0 and 10⁵CFU/ml bacteria were plated onto plates containing ABIM medium plus AS at 50 μM, DIMBOA at 100 μM plus kanamycin at 50 μg/ml. Colonies that grew on this selection medium were subsequently assayed for growth on plates containing kanamycin but no phenolic. Colonies that grew under these conditions are constitutive for kanamycin resistance and were not characterized further, while colonies that could not survive in this screen were characterized further.

B. Results

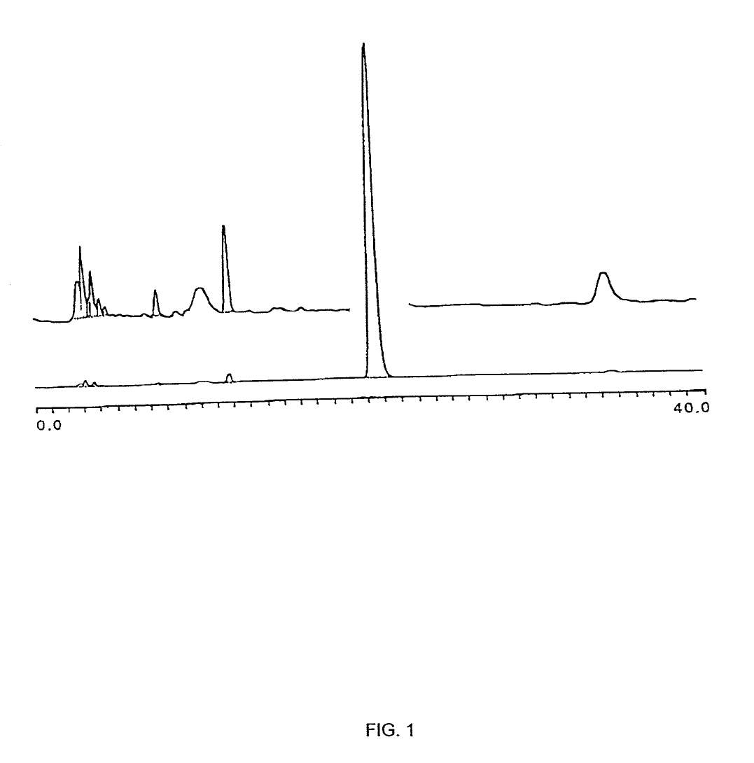

The organic exudate of Zea mays seedlings was evaluated by RP-KPLC in FIG. 1 and found to consist essentially of a single compound, >98% of the organic constituents. ¹H-NMR (acetone-d₆) of this exudate showed two methyl singlets, δ3.78 (3H) and 3.91 (3H), a 1,2,4 substituted aromatic, δ7.16, 1H, d, J=8.8 Hz, δ6.68, 1H, d, J=2.5 Hz, δ6.70, 1H, dd, J=2.5, 8.8 Hz, and a broad singlet at δ5.4 (1H). The IR (CHCl₃) spectrum contained a carbonyl stretch at v 1686 cm⁻¹ and a broad OH stretch at v 2958 cm⁻¹. MS (EI, 70 eV) gave a possible molecular ion at m/z 225 which was confirmed both by the M+H ion, m/z 226, obtained by CI (isobutane) analysis and by acetylation (Ac₂O, pyridine) to gave a monoacetate, CI (isobutane) m/z 268 (M+H). Exact mass measurements on the molecular ion established a molecular formula of C₁₀H₁₁NO₅. Taken together, these data were most consistent with an aromatic ring fused to a six-membered lactam. Placement of the additional methoxy group on the N atom suggested structure l. Final proof was obtained via methylation of 2,7-dihydroxy-4-methoxybenzoxazin-3-one (DIMBOA) isolated from the same maize seedlings.

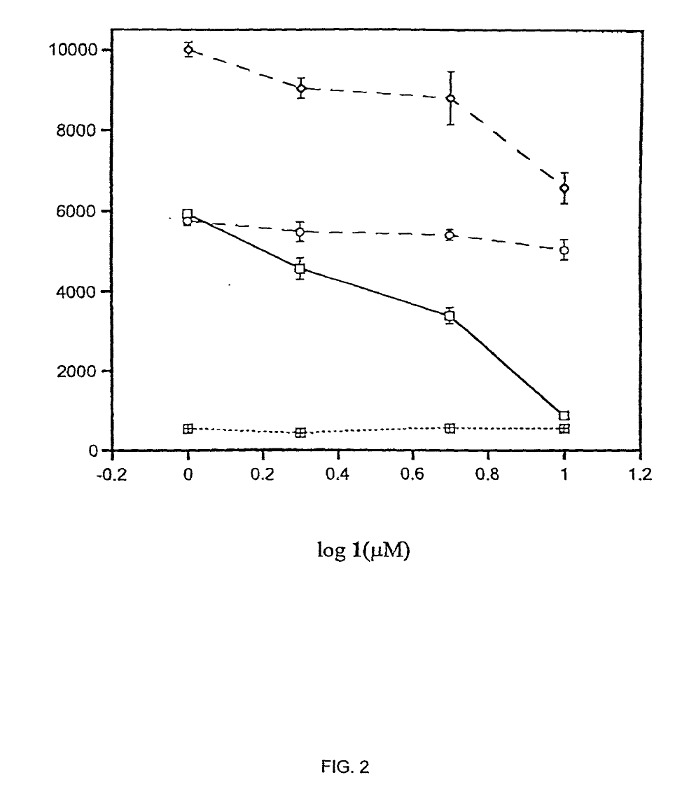

The capacity of 1 to inhibit vir gene expression was tested by monitoring β-galactosidase activity in an A. tumefaciens strain carrying wild-type vir induction genes and a lacZ gene under the control of a vir promoter (Hess et al., 1991). I strongly inhibits expression induced by 100 uM acetosyringone, AS, and the inhibition was shown to be specific to vir activation by experiments demonstrating induction of the occ regulon of the same plasmid was unaffected (data not shown). A more precise test for specificity was available via the utilization of a plasmid containing a virA gene, the putative AS receptor, which contitutively expressed the vir genes in the absence of AS. As shown in FIG. 2, this constitutively activated VirA strain functions independently of the [1]. Therefore, the inhibition by 1 occurs upstrean, of VirA activation.

Related benzoxazinones are known and found in several of the Gramineae, including maize, and DIMBOA itself has been shown to inhibit A. tumefaciens. Hofman et al., 1970; Gambrow et al., 1986; Herdin et al., 1993; Sahi et al., 1990. DIMBOA however exists in the vacuole of intact plant cells as the inactive glucoside, and only vacuole rupture exposes the glucosidase capable of releasing the toxic aglycone. In contrast, I is uncharged at neutral pH and is excreted in large quantities along the root surface, that part of the seedling most directly exposed to the soil-borne bacterium.

Curiously, 1 appears the most hydrolytically labile of this class of structures, with a measured t_{1/2}<4 hr at pH 5.5, and −10 mm at pH 7. Under the assay conditions, 1 decomposes cleanly to the inactive 6-methoxy-2H-benzoxazolin-2(3H)-one, and based on these decomposition rates during the 8 hr assay period, the actual IC₅₀ is in the nanomolar range. I becomes the most potent and specific inhibitor yet discovered for any environmental sensing two-component signal transduction system. Moreover, from estimates of the surface area per seedling, the concentration of 1 around the radicle of one-week-old maize seedlings should be between 1 M and 10 mM, depending on whether a 10 μ or a 100 μ volume element is used to define the surface hydration. Therefore, a steep concentration gradient of 1 can be expected to exist around the root surface (Fate et al., 1996), and vir gene expression of any surface localized A. tumefaciens colonies would completely inhibited within this specific zone. This inhibitory zone readily explains the observed difficulty transforming this organ with the bacterium.

### Example 2

Characterization of Phenolic Induction in Agrobacterium

A. Materials and Methods

Proton (¹H) NMR spectra were either obtained on the U. of Chicago DS1000 spectrometer that was equipped with a Nicolet 1280 data acquisition system operated at 500 MHz, or a General Electric (GE) QE300 spectrometer operating at 300 MHz. Carbon (¹³C) NMR spectra were obtained on a General Electric QE-300 spectrometer operating at 75 MHz.

Two-dimensional (2-D) NMR spectra were obtained on a General Electric (GE) GN-500 spectrometer that was equipped with the Omega Data system. Low-resolution mass spectra (MS) were obtained using a UG 70-250 Mass spectrometer. High pressure liquid chromatography (HPLC) system was a Rainin system which included Model HPXL pumps (pressure module max; 8700 psi), Dynamax Absorbance detector (model UV-D) and a Macintegrator software installed into the MacElus computer. Ultraviolet (UV) spectra were recorded with a Perkin-Elmer lambda 6 UV/VIS spectrophotometer in quartz cuvettes with an optical pathway of 1 cm. Circular dichroism (CD) spectroscopy was measured using a Jasco-600 CD spectrophotometer. Optical rotation was measured with a Perkin-Elmer 141 polarimeter. Ozonolysis is carried out with a Welsbach motel T-408 laboratory ozonier, Welsbach Corp., Philadelphia, Pa.

Syntheses. Acetosyringone (AS), acetovallinone (AV), guaiacol, 4-methyl-2-methoxy-phenol, and 5,6-dimethoxy-1-indanone were either purchased from Aldrich and Lancaster Chemical Companies and were used directly. The syntheses of dehydrodiconiferyl ferulate, 3-(4′-hydroxy-3′-methoxyphenol)-butan-1-ol, and 2-phenoxy-2-(4′-hydroxy-3′-methoxyphenyl)-ethanol were completed as previously reported by Hess et al. Hess et al., 1991. HPLC grade solvents were purchased from Fisher Chemical Co. or J.T. Baker Chemical Co. Tetrahydrofuran (THF) and diethyl ether were distilled from sodium/benzophenone and dichloromethane (CH₂C₁₂) was distilled from calcium hydride. H₂O was double distilled from all glass stills.

Flash column chromatography employed EM Science silica gel, 230-400 mesh, 40-63 Å in diameter. Thin layer chromatography was performed with Baker-flex silica gel IB2-F plastic backed plates with a 0.25 mm silica gel layer. Plates were visualized by 254 nm illumination, and/or treatment with a ceric ammonium sulfate/trichloroacetic acid.

Synthetic methods and structural characterization of 2-methoxy-4-ethyl-phenol, 2-methoxy 4-isopropyl-phenol and 4-(tert-butyl)2-methoxyphenol are described in Supplementary materials.

trans-4-Benzyloxy-3-methoxy cinnamyl alcohol. Trans-4-hydroxy3methoxy-cinnamic acid was esterified (MeOH, SOCl₂) and benzylated to afford the methyl trans-4-benzyloxy-3-methoxy cinnamate. A dried three necked flask equipped with an additional funnel and under N₂ was charged win lithium aluminum hydride (LAH) suspended in THP and cooled in an ice bath for 15 minutes before a solution of methyl cinnamate in THF was added dropwise with stirring.

After 15 mm the reaction mixture wets partitioned between EtOAc and dilute aqueous HC1 and the organic phase was washed with brine, dried with sodium sulfate, concentrated and chromatographed (SiO₂, hexanes: EtOAc, 7:3) to give trans-4-benzyloxy-3-methoxy cinnamy alcohol as a yellow oil.

(+)- and (−)-trans-[3-(4-Hydroxy-3-methoxy-phenyl)-cyclopropyl]methanol (trans-CP). The enantiomers of dioxaborolane were prepared by refluxing the corresponding N, N, N′, N′-tetramethyltartaric acid diamide with 1-butaneboronic acid in anhydrous toluene in a Dean-Stark trap for 15 h. A 100 ml flask charged with 30 ml distilled CH₂Cl₂ was cooled to is −25° C. before 8.9 ml (8.9 mmol) of a 1.0 M solution of diethyl zinc in hexanes, 0.93 ml (8.9 mmol) of ethylene glycol dimethyl ether, and 1.43 ml (17.8 mmol) of diiodomethane were added and stirred for 10 mm. 0.48 g (1.78 mmol) of the protected cinnamyl alcohol and 0.53 g (1.95 mmol) of the chiral dioxaborolane were added in 10 ml CH₂Cl₂, and the reaction mixture was stirred for 3 h, quenched with 50 ml saturated NH₄CI, and the aqueous portion was extracted with diethyl ether. The combined organic layers mere stirred vigorously for 12 h with aqueous KOH (5 M), and the organic layer was then successively washed with 10% aqueous HCI, saturated aqueous NaHC0₃, H₂0, saturated aqueous NaCl, and dried, concentrated in vacuo and chromatographed on SiO₂ (1:4, hexane/EtOAc) to afford 0.35 g (69%) of the desired cyclopropylmethanol. ¹H NMR; δ(CDCl₃) 7.43—7.30 (m, 5H), 6.78 (d,2H), 6.65 (d, 1H), 6.56 (dd, 1H), 5.12 (s, 2H), 3.88 (s, 3H), 3.61 (m, 2H), 1.77 (my 1H), 1.53 (b, 1H), 1.38 (m, 1H), 0.90 (m, 2H); ¹³C NMR, δ (CDCl₃) 149.5, 146.2, 137.3, 135.6, 128.4, 127.7, 127.2, 117.6, 114.3, 110.2, 71.2, 66.5, 55.9, 24.8, 20.9, 13.3. Trans[3(4-benzyloxy-3-methoxyphenyl)-cyclopropyl]methanol underwent hydrogenolysis to afford after chromatography (SiO₂, 1:1, hexane/EtOAc) either (+)- or (−) trans-CP. ¹H NMR; δ (CDCl₃) 6.80 (d,2H:), 6.61 (d, 1H), 6.56 (dd, 1H), 5.57(s, 1H), 3.59 (m, 2hH, 1.77 (m, 1H), 1.65 (b, 1H), 1,38 (m, 1H), 0.88 (m, 2H); ¹³C NMR; δ (CDCl₃) 149.1, 146.3, 134.1, 118.4, 115.2, 109.0, 66.5, 55.8, 24.6, 21.0, 13.1.

5-hydroxy-4-methoxy-1-indanone (E-indanone). A 50 ml three-neck flask fitted with a vacuum line, a nitrogen line and a H₂-filled balloon was charged with 2,3-dimethoxycinnamic acid, 3.0 g (0.014 mmol), 10% palladium/carbon catalyst (3 spatula tips) and 20 ml of absolute ethanol was stirred vigorously. The flask was evacuated and flushed with N₂ three times, filled with H₂ and after 40 mins the Pd/C catalyst was removed through a short column of celite, and the solvent evaporated in vacuo to afford 2,3-dimethoxyphenylpropanoic acid in 97% yield. ¹H NMR (300M Hz, CDCl₃) δ7.01 (d, IH, J=7.9 Hz, aromatic). 6.81-6.84 (m, 2H, aromatic), 3.88 (s, 6H, 2 OCH₃), 2.99 (t, 2H, J=7.7 Hz), 2.69 (t, 2H, J=8.2 Hz). Mass spectrum (EI⁺70 eV)m/z 210 (M⁺, 100%), 164(30%), 151 (44%), 136(46%), 91 (61%), 77 (35%), 65 (24%).

A IL beaker containing 50 g of polyphosphoric acid (PPA) was heated over a steam bath to 90° C. before 2,3-dimethoxyphenylpropanoic acid, 2.0 g (9.52 mmol), was added in one portion and stirred vigorously with a glass rod for 3 min (the temperature should remain around 90° C.). Another 50 g of liquefied PPA was added and the mixture was warmed with vigorous stirring for another 4 mm, cooled to 60° C. with 150 g of crushed ice and stirred until a yellow oil has separated. The resulting mixture was extracted three times with ether, the combined organic fractions washed with 5% NaOH and water, dried over anhydrous sodium sulfate and evaporated in vacuo to afford 4,5-dimethoxy-1-indanone as a yellowish oil in 86% yield. ¹H NMR (300 MHz, CDCl₃) δ7.51 (d, 1H, J=8.5 Hz, aromatic), 7.01 (d, 1H, J=8.6 Hz, aromatic), 6.36 (s, 1H, —OH), 4.02 (s, 3H, —OCH₃), 3.98 (s, 3H, —OCH₃), 3.08 (t, 2H, J=6.1 Hz, —CH₂—), 2.71 (t, 2H, J=5.8 Hz, —CH₂—). ¹³C NMR (75 MHz, CDCl₃) δ198.54, 158.67, 149.63, 145.91, 132.33, 120.87, 116.11, 60.49, 58.99, 36.37, 23.41. Mass Spectrum (EI⁺, 70 eV) m/z 192 (100%), 177 (34%), 149 (9%), 107 (9%), 91 (6%), 77 (7%).

A 50 ml, 2-neck flask equipped with condenser and nitrogen line was charged with 4,5-dimethoxy-1-indanone, 1.5 g (7.80 inmol), and NaCN, 1.91 g (0.038 mol), dissolved in 15 ml of dry DMSO and heated in an oil bath to 160° C. with vigorously stirring for 20 hours. The dark brown reaction mixture was quenched with H₂O, 150 ml, the aqueous layer extracted four times with ether, and the combined organic layer dried with anhydrous Na₂SO₄ and concentrated in vacuo to a dark oil. 5-hydroxy-4-methoxy-1-indanone, E-indanone, was obtained as yellow crystals in 71% yield after column chromatography (SiO₂, 9:1, hexane/EtOAc). ¹H NMR (300 MHz, CDCl₃) δ7.49 (d, 1H, J=8.2 Hz, aromatic), 7.00 (d, 1H, J=8.2 Hz, aromatic), 6.36 (s, IH, —OH), 4.01 (s, 3H, —OCH₃), 3.20 (t, 2H, J=5.7 Hz, —CH₂—), 2.71 (t, 2H, J=6.0 Hz, —CH₂—). ¹³C NMR (500 MHz, CDCl₃) δ204.70, 154.01, 145.28, 143.12, 131.21, 120.70, 115.95, 60.25, 36.20, 23.23. Mass Spectrum (EI⁺, 70 eV) m/z 178 (M⁺, 100%), 163 (50%), 135 (21%), 107 (28%), 91(23%), 77 (16%).

5-hydroxy-6-methoxy-1-indanone(Z-indanone). Demethylation of 5,6-dimethyl-1-indanone followed the saline course as the E-indanone to yield a pure yellowish oil in 67% yield. ¹H NMR (300 MHz, CDCl₃) δ7.21 (s, 1H. aromatic), 6.98 (s, 1H, aromatic), 6.23(s, 1H, —OH), 3.95 (s, 3H, —OCH₃), 3.05 (t, 2H, J=5.27 Hz, —CH₂—). 2.68 (t, 2H, J=5.83 Hz, —CH₂—), ¹³C NMR (500 MHz, CDCl₃) δ205.95, 152.42, 150.92, 146.83, 129.59, 111.10, 104.02, 56.16, 36.45, 25.38. Mass Spectrum (EI⁺, 70 eV) m/z 178 (M⁺, 100%), 163 (62%), 135 (16%), 107 (24%), 91 (52%), 77 (18%), 63 (9%), 55 (7%).

Bacterial Strains and Media. E. coli strain DH5α (Gibco) was used as recipient of plasmids from all cloning manipulations. These plasmids were then isolated and transferred to A. tumefaciens, by electroporation, as necessary. A. tumefaciens strains: A348 (Garfinkel et al., 1981) has the C58 chromosomal background with pTi6; 358mx (Stachel et al., 1986) is an A348 derivative harboring insertion of the transposon Tn3-HoHol in the virE2 gene of pTiA6 creating a virE::lacZ fusion. UIA143 is strain C58 lacking its Ti plasmid and carrying an erythromycin resistance gene in the chromosomal recA gene. Farrand et al., 1989. Strains AB 140-148 are phenolic hypersensitive mutants, derived from srain A348 as described below.

Strains were maintained on LB plates or AB minimal medium supplemented with antibiotics as appropriate. Winans et al., 1988. AB induction medium (ABIM), pH 5.5 and containing 1% glucose or 1% glycerol ±0.1 % arabinose, was used for vir gene expression studies. The plasinids used in this study are listed below.

TABLE 9

| Plasmid | Relevant characteristics | Source or reference |
|---|---|---|
| pAC1 | NPTII structural gene (nptII) from | this study |
| | pCamVNeo as a BamHI fragment | |
| | cloned into BamHI site of pED32, | |
| | Tet^{R} | |
| pAC2 | virB^{P}/nptII from pAC1, as a JomdIII | this study |
| | fragment, cloned into pMON596; | |
| | Spec^{R}, IncP | |
| PCcamVNeo | pUC8 derivative containing the | Fromm et al., 1986 |
| | nptII coding sequences on a 1.0 kb | Beck et al., 1982 |
| | BamHI fragment | |
| pCF218 | traR expressed constitutively from | Fuqua & Winans, |
| | tetR- promoter; cloned into | 1994 |
| | pSW213, Tet^{R}, IncP | |
| pED32A | VirB promoter expression vector; | Ward et al., 1990 |
| | Tet^{R}, Amp^{R}, Spec^{R}, IncP | |
| PMON596 | binary vector; carries the 19S- | Ma et al., 1992 |
| | hygromycin resistance gene; Spec^{R}; | |
| | IncP; derived from pMON574 | |
| pSW209 | IncP, virB::lacZ fusion derived from | S.C. Winans |
| | pSM243cd, Kan^{R}. | |

pAC2 construction. The BamHI fragment from pCamVNeo (Fromm et al., 1986), which contains the coding sequence of nptII, was cloned into the BamHI site of pED32, a plasmid that contains the virB promoter win a multiple cloning site (Ward et al., 1990). This yielded pAC1, a plasmid that had nptII expression driven by the inducible virB promoter (virB^{P}/nptII), with tetracycline resistance as its antibiotic maker. The HindIII fragment carrying the virB^{P}/nptII construct was removed from pAC1 and inserted into the HindIII site of pMON596. This yields a plasmid, pAC2, that is spectinomycin resistant, but sensitive to kanamycin unless vir-gene inducing phenolic compounds are present (see Results).

vir Induction Assay. A. tumefaciens strains were grown overnight at 27° C. to an optical density of about 0.3-0.6 at 600 nm (OD₆₀₀) in LB medium supplemented win appropriate antibiotics. Cells were then pelleted by centrifugation and resuspended to an OD₆₀₀ of 0 1 per ml in sterilized induction medium (ABIM. pH 5.5) supplemented with either 1% glucose or 1% glycerol with or without 0.1% arabinose (w/v). The verious phenolic compounds tested were dissolved in a minimal amount of DMSO in water as mM stocks, and diluted with ABIM to the desired concentration (final DMSO concentration was no greater than 0.1%). The bacteria were incubated with at 28° C. for 8-16 hours and subsequently assayed for β-galactosidase activity by the method of Miller, 1972. ED₅₀ values for the inducers evaluated were estimated from such dose response plots. Each assay value reported is the mean of three replicates; error bar indicates one standard deviation from the mean. Data are representative of three similar independent experiments.

Mutant Selection. Preliminary experiments indicated that A. tumefaciens strain A348/pAC2, grown on induction plates plus 40 μg/ml kanamycin survive at 100 μM AS but are killed at 30 μM AS, demonstrating the phenolic induction of kanamycin resistance is provided by pAC2 (vector controls died at all AS concentrations). Phenolic hypersensitive mutants were selected as follows. Individual colonies of A348/pAC2 were grown overnight in LB broth plus spectinomycin, and the progeny from each colony kept separate to ensure that each mutation was an individual event, not part of a clonal population. The bacteria were subsequently washed in ABIM medium, resuspended to an OD₆₀₀ of approximately 0.1 and induced overnight in ABIM media plus 100 μM AS. The next day the cells had grown to an approximate OD₆₀₀ of 1.0 and 2×10⁵ to 2×10⁶ bacteria were plated onto plates confining ABIM medium containing 10 μM AS and 40 μg/ml kanamycin. Colonies that grew on this selection medium were subsequently assayed for growth on plates containing kanamycin but no phenolic. Colonies that grew under these conditions are constitutive for kanamycin resistance and were not characterized further, while colonies that couldn't survive in this screen were characterized further.

The kanamycin resistance test of selected colonies in response to different doses of phenol was done in the following manner: after one night of induction in ABIM over a range of inducer concentrations, as described above, the bacteria were washed and subcultured into ABIM at a starting OD₆₀₀ of approximately 0.1. The ABIM included phenolics at a range of concentrations between 0.1-1000 μM. At each concentration of inducer three cultures have kanamycin at a concentration of 20 μg/mL, while one culture has no kanamycin. After overnight culture the OD₆₀₀ is determined and kanamycin resistant growth as a percentage of control is determined using the following calculation:

$$\% growth = \frac{( {OD}_{600} overnight culture + kanamycin ) \times 100}{( {OD}_{600} overnight culture w / no kanamnycin )}$$

The mean of three samples is then plotted versus log of concentration of inducer.

Immunoblot. Cultures of different strains were induced overnight in ABIM containing 1% glycerol and 0.1% arabinose and varying phenolic concentrations. Samples from each culture, containing an equivalent OD₆₀₀, were denatured by boiling in sample buffer for 3-10 minutes, and 10 μL from each sample was loaded onto 10% acrylamide gels and separated via SDSpolyacrylamide gel electrophoresis (PAGE). Proteins were transferred from the acrylamide gel to nitrocellulose purchased from Schleicher and Schell (Keene, N. H.) by electrotransfer. VirB 10 expression was examined using immunoblot procedures and antibodies described in Beaupre et al., 1997.

Virulence Assays The capacity of various strains of A. tumefaciens to induce tumors on leaf explants of Nicotiana tabacum cv Havana 425 was tested as described previously. Banta et al., 1994.

Ti plasmid conjugation Conjugal transfer of pTiA6 from either the wild-type or hypersensitive strains to strain UIA143/pSW2O9 was carried our as described by Bohne et al., 1998.

B. Results

Stereochemical Evaluation of Xenognostic Phenols. While several structural regions of the xenognostic phenols are known to be critical for activity (Duban et al., 1993; Melchers et al., 1989); Spencer & Towers, 1988), only one of the known inducers is chiral, the aglycone of the plant cell growth promoting dehydrodiconiferyl glucoside (DCG) (Teutonico et al., 1991; Orr et al., 1992; Lynn et al., 1987; Binns et al., 1987; Tamagnone et al., 1998). In initial attempts to address the chiral dependence at this position, the limits of substitution at the benzylic chiral center were evaluated by testing the capacity of certain analogs to induce expression of a virE::lacZ reporter construct. Little change in vir gene expression was observed when the alkyl substituent was changed from —H to —CH₃. However, both the maximal induction was reduced and half-maximal effective concentration was increased as the benzylic center was clanged from 2°, to 3°, to an inactive 4° carbon. Importantly, these structural changes resulted in no significant alteration in the pKa of the phenol or the required assay conditions, demonstrating that sterically demanding substituents at this center critically compromise the inducing activity of the phenol.

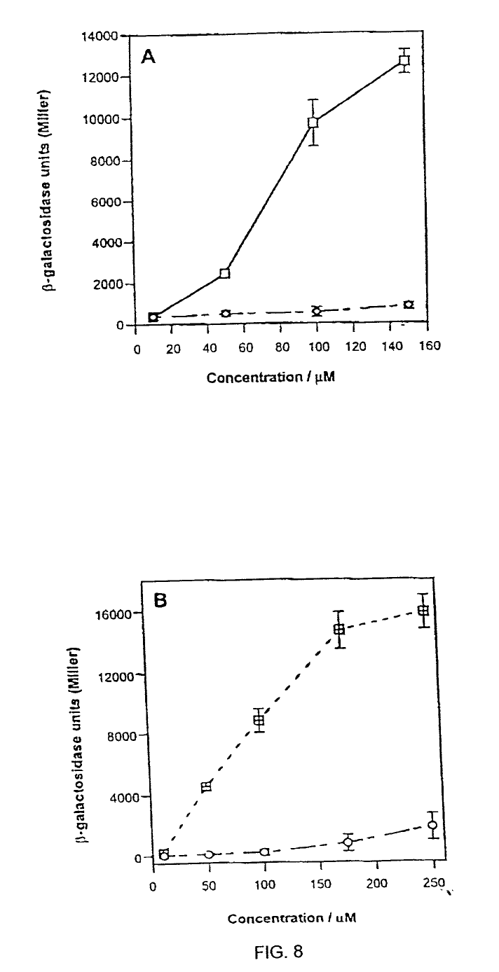

Given these structural requirements on substituents at the para-alkyl center, the stereospecificity of the DCG aglycone, DCA, was examined by analysis of the more readily prepared (±)-trans-dehydroferulate dimethylester (DDF). Oxidative dimerization of methyl ferulate gave the desired carbon skeleton and the two trans-DDF enantiomers were resolved by chiral HPLC. The absolute stereochemistry of each isomer was assigned by conversion to the DCG aglycone (Orr et al., 1992; Dudley, 1991) whose absolute configuration has been assigned (Hirai et al., 1994). Analysis of the expression of a virB::lacZ reporter fusion showed that (2R,3R)-(−)-trans-DDF possessed all the inducing activity of the racemic mixture, at least up to a concentration of 250 μM (FIG. 8A). While this data suggested an absolute stereochemical requirement for vir induction, DCA is susceptible to benzofuran ring-opening to the stilbene derivative, a ring-opening expected to be even more pronounced in trans-DDF. In fact, mild base treatment of the diester gave the stilbene product, a structure which did induce vir gene expression under the assay conditions used. However, attempts to detect the stilbene by HPLC of the bacterial culture during vir induction were unsuccessful with both compounds.

To address whether stereospecific decomposition to the stilbene accounted for the vir inducing activity, DCA was condensed to the simplest structure that (i) retained the critical phenol for vir inducing activity (Duban et al., 1993), (ii) maintained the three dimentional configuration of the two chrial centers of trans-DCA, and (iii) suppressed the ring-opening pathway. With the emergence of stereo selective reagents for the construction of chiral three membered carbocycles, general routes for the preparation of the appropriate trans-cyclopropanes were developed. Aranti, 1985; Noyori, 1990; Salomon & Koch, 1973; Lownthal et al., 1990; 1991; Evans et al., 1991; 1992. Both trans- and cis-isomers were prepared via the appropriately substituted styrene, ethyl diazoacete and the bis[(4S)-oxaline] copper (II) catalyst. Lownthal et al., 1990; 1991. The predicted isomers, (1R,2R)-trans-1-hydroxymethyl-2-(4-hydroxy-3-methoxyphenyl)cyclopropane (cis-CP) were obtained in over 90% enantiomeric excess (ee) as determined by chiralcel OD HPLC.

Confirmation of the absolute stereochemical assignment was obtained both by coupled oscillator analysis of the dibenzoates (Harada et al., 1972; Harada & Nakanishi, 1983) of the trans-CP isomers as well as by an alternate enantioselective synthesis. Charrette workers (Charette & Juteau, 1994; Charette et al., 1995; Theberge et al., 1996) have demonstrated predictable stereoselective cyclopropanation using dioxaboralane enantiomers as directing groups in the Furukawa reaction (Simmons & Smith, 1958; 1959; Furukawa et al., 1966;1968. Cyclopropanation of the protected trans-4-benzyloxy-3-methoxy-cinnamyl alcohol via this stategy indeed resulted in the efficient, enantiosclective (89%-92% ee) preparation of trans-CP isomers, further establishing the absolute stereochemical assignment.

Each cis and trans enanantiomer of CP were tested for their ability to induce vir gene expression. In the case of cis-CP, the (+) and (−) enantiomers showed equivalent activity. The chiralcel OD purified (+)-trans-CP, however, accounted for virtually all the activity of the trans racemate (FIG. 8B). Significantly, the three dimensional arrangement of the substituents on the cyclopropyl centers of the active (+)-trans-CP are identical to both the active DCA and DDF, where the ester replaces the hydroxymethyl substituent, isomers. The cyclopropyl ring is also significantly more stable to ring-opening than the benzofuran of either DDF or DCA. These results demonstrate that chiral specificity at centers distant to the OH can be critical in phenol-mediated induction of vir gene expression, and provide support for the hypothesis that a specific receptor, or receptors, must be involved in signal perception.

Isolation of AS Hypersensitive Mutants. To further evaluate the implicated specific phenol receptor in Agrobacterium that mediates expression of the virulence genes, a novel genetic screen was devised to isolate mutants with altered inducer sensitivity. pAC2, a broad-host-range plasmid containing the neomycin phosphotransfer (nptII) gene under the control of the virB promoter (Ward et al., 1990) was constructed. This plasmid confers on the wild-type A. tumefaciens strain A348 the capacity to grow in medium containing kanamycin only when a sufficient concentration of vir-inducing phenol is present. The selection strategy was to incubate large numbers of A348/pAC2 cells on agar solidified plates containing IM, 40 μg/ml kanamyin and 10 μm AS, a concentration that is ten-fold lower than is required by these cells to survive in the presence of kanamycin. Two classes of surviving colonies were recovered, constitutive and hypersensitive mutants, easily distinguished by patching the colonies onto medium containing kanamycin but lacking AS. Using this strategy, nine hypersensitive mutants were isolated from a total of greater than 2×10⁷ cells exposed to the selection conditions.

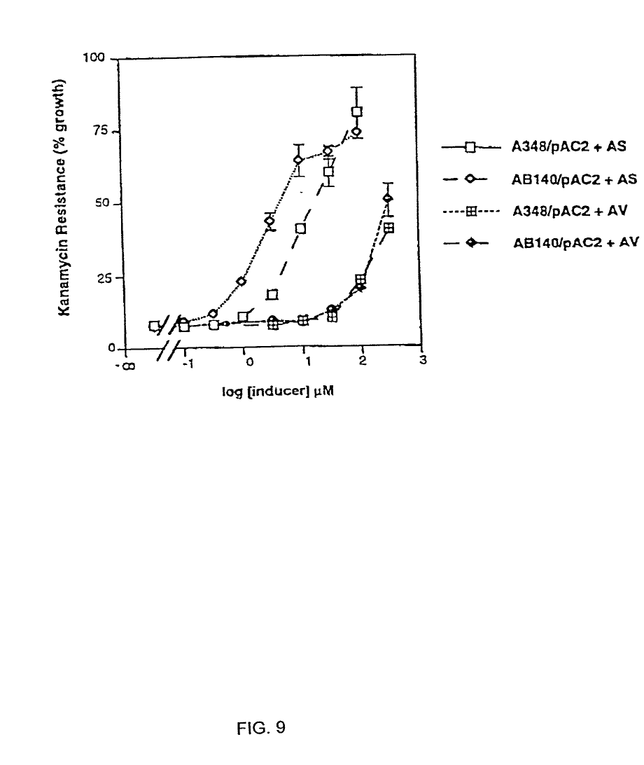

The hypersensitive nature of the mutants was confirmed by evaluating their dose response to AS. For example, mutant AB140 was tested for kanamycin resistance in liquid medium over a range of AS concentrations. As shown in FIG. 9, AB140 grows in the presence of kanamyin at approximately one-half log lower concentration of AS than does wild-type. AB140 shows no basal level induction of vir gene expression, as judged by growth in kanamcyin containing medium in the absence of AS, demonstrating that this is not a constitutive mutant. Intriguingly, this mutant, and all others tested thus far, are not hypersensitive to methoxyphenols, e.g., AV, indicating that the observed change in phenol sensitivity is specific to the dimethoxyphenols.

To ensure that the mutants hypersensitive to AS were a result of a change in the vir gene induction pathway rather than a mutation in the reporter plasmid pAC2, other methods to analyze vir gene induction and the virulence response were investigated. First, all nine mutants were transformed with pSW209 carrying a virB::lacZ reporter construct. The β-galactosidase activities of all strains were hypersensitive in an AS dose-response study. The observed changes are not due to some artifact of the reporter plasmids as the expression of VirB10, one of the proteins of the virB operon on its Ti plasmid, in cells of AB140 was detectable after induction with 0.3 μM AS. Wild-type cells required 1 μM AS to produce detectable VirB10. Thus, the mutants are hypersensitive to AS when assaying either the virB^{P}/nptII reporter, the virB::lacZ reporter, or directly assaying expression of VirB10 from the native viral promoter on the Ti plasmid. These results demonstrate that the mutation affects some aspect of the phenol-mediated induction of vir expression.

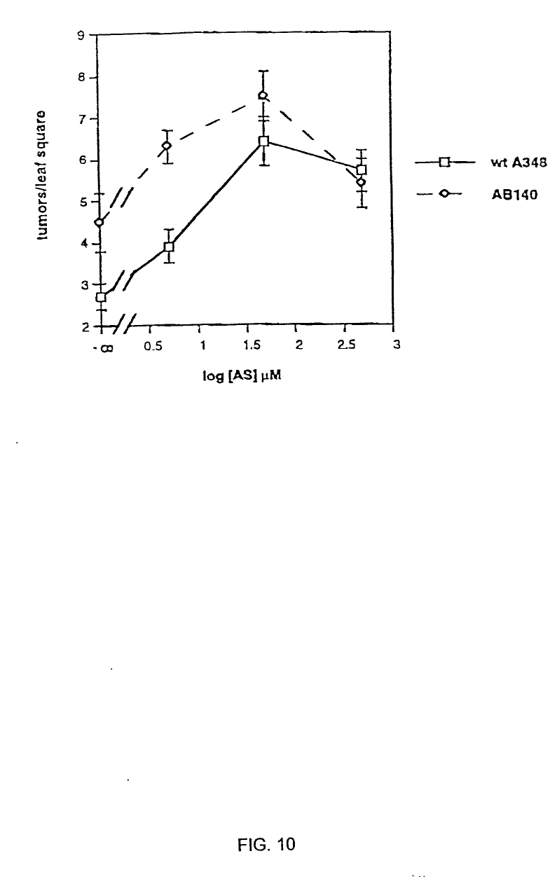

A crucial question is whether the mutants that demonstrate hypersensitivity to the xenognostic phenols are also “hypervirulent.” The virulence of the strains was assayed by monitoring their capacity to induce crown gall tumors in the tobacco leaf assay. In this assay, bacteria and leaf explants are co-cultivated for two days at varying levels of AS and then transferred to antibiotic containing medium that stops bacterial growth but allows plant tumor growth. Banta et al., 1994. In the case of wild-type strain A348, some tumor formation is observed on the leaf explants co-cultivated in the absence of AS in the co-cultivation, as expected because of xenognostic phenols released from the wounded cells (FIG. 10). As the concentration of the phenol in the co-cultivation medium was increased, the number of tumors formed per infected leaf explant significantly increased. The hypersensitive mutant AB140 induced significantly more tumors than wild-type in the absence of AS in the co-cultivation medium, and, most importantly, reached its maximal capacity to induce tumors at a log order lower concentration of AS in the co-cultivation medium (FIG. 10). Importantly, the maximum number of tumors formed by both the wild-type strain and AB140 was approximately the same, consistent with the observation that AB140's hypersensitivity is not a result of simple overexpression of the vir genes. Thus, the hypersensitive response for vir expression demonstrated using the reporter assay is matched by AB140's ability to induce tumor formation at low concentration of xenognosin.

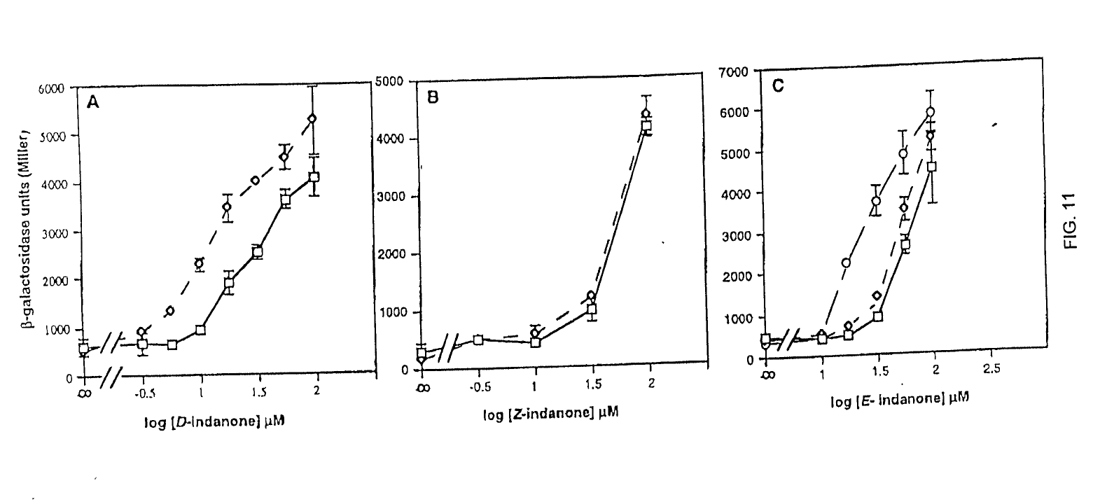

Structural Specificity. The selected mutants exhibited the same stereospecificity as the wild-type strain. The (+)-trans-CP accounted for all the inducing activity of the racemate and the mutants appeared at best slightly hypersensitive to the active enantiomer. The free rotation of the para benzylic substituent in AV, AS, as well as the CP and DDF enantiomers raises the possibility the geometric isomers relating the orientation of that group to the ring methoxy substituent could exist in the receptor-bound xenognosins. To evaluate this possibility, the rotation of the aryl-carbonyl bond was locked via the construction of isomeric indanones. The inducing activity of the 5-hydroxy-4,6-dimethoxyindanone (D-indanone) was little changed from is AS in a wild-type background, and like AS, AB140 was hypersensitive to this compound (FIG. 11A).

The E-indanone, 5-hydroxy4-methoxyindanone, constrains the carbonyl oxygen and the methoxy on opposite sides of the phenol-alkyl axis, whereas the Z-indanone, 5hydroxy-6-methoxyindanone, positions them on the same side of this axis. Wild-type strain A348 responds to the isomeric indanones with equivalent activity, however AB140 is hypersensitive only to the E-indanone (FIG. 11B and FIG. 11C). The E-indanone does not account for all the hypersensitivity of the D-indanone in AB140, but it does so in a second hypersensitive strain, AB144 (FIG. 11C). Similar results were observed with strains AB146 and AB147, all the hypersensitivity of AB146 to the E-indanone was accounted for by the E-indanone while the hypersensitivity of AB147 was only partially accounted for by the E-indanone. Curiously, only the E-indanone was responsible for any of the hypersensitivity in the strains thus far analyzed.

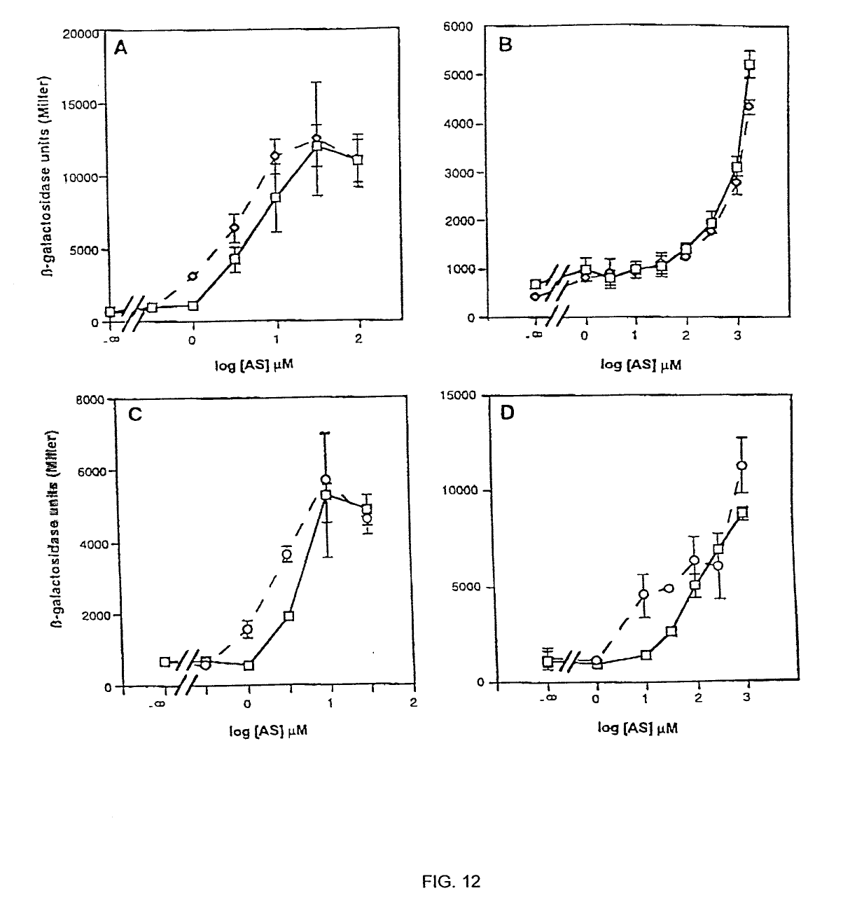

Effect of Sugars on Phenol Hypersensitivity. The results of Peng et al., 1998, demonstrate that both phenol sensitivity and specificity are dramatically affected by the presence of inducing sugars and are attributable to the ChvE-VirA interaction. This result suggests that a possible mechanism for the change in sensitivity and specificity described here exists within the sugar perception and signaling system. Therefore, the phenol hypersensitive strains were tested in the presence or absence of 0.1% arabinose, and by this analysis the mutants fell into two classes. The first, characterized by strain AB140, exhibits a sugar dependent hypersensitive phenotype (FIG. 12A and FIG. 12B). Only when arabinose was present in the induction medium was the sensitivity to AS increased in comparison to wild-type strain A348. The second class of mutants, characterized by strain AB147, exhibits a sugar-independent hypersensitive phenotype (FIG. 12C and FIG. 12D). Even in the absence of arabinose, these strains are hypersensitive to AS, responding to AS at a half-log lower concentration than does the wild-type strain. Typical of this class, AB147 is more sensitive to AS in the presence of arabinose than in its absence (FIG. 12C and FIG. 12D), indicating that they still are responding to the ChvE-sugar signaling system.

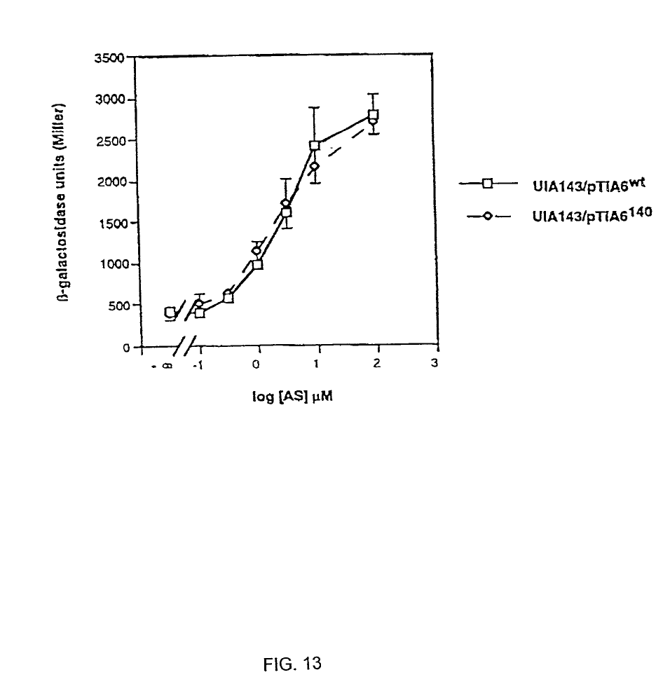

Genetic Characterization. To determine whether the gene responsible for phenol hypersensitivity of the mutants was virA or was located on the Ti plasmid, the Ti from both wild-type A348 and the mutants were moved into strain UIA143 by conjugation. UIA143 has the same chromosome as both A348 and the mutants, but lacks a Ti plasmid. Transconjugants carrying either the wild-type pTiA6 or AB140's Ti responded identically to AS for vir induction (FIG. 13). The Ti plasmids of five additional hypersensitive mutants, both sugar independent and sugar dependent, were conjugated into UIA143, and in all cases, the transconjugants exhibited wild-type sensitivity to the xenognostic phenols. These results indicate that mutations in virA are not responsible for the hypersensitive phenotype, and, additionally, demonstrate that no other gene localized to the Ti plasmid, including virG, is responsible for the phenotype. The only other gene known to be critical in the phenol induction pathway is chvE. However, sequence analysis revealed that chvE genes from both sugar dependent and sugar independent strains are also wild-type. Thus, the gene responsible for the phenol hypersensitive phenotype must be a novel component in the xenognosin response system.

C. Discussion

More than 80 different phenols are able to induce virulence in Agrobacterium tumefaciens, and this broad structural diversity best be explained by a vir induction mechanism that exploits a general physical effect of the phenol rather perception through a specific receptor. However, the findings that only a single enantiomer of the chiral inducers, (−)-trans-DDF and (+)-trans-CP, are active, that the three dimensional arrangements of the substituents on these molecules are identical, and that no stereospecific metabolism or uptake of the different chiral isomers could be detected, argue that indeed an enantiospecific receptor must exist.

Further proof for the existence of this receptor(s) was found through the construction of pAC2 carrying a nptII gene under the control of the virB promoter. This plasmid offered a novel selection strategy in which survival in kanamycin containing medium at low [AS] could only occur in mutants that either had increased sensitivity to AS or were constitutive for vir gene expression. Earlier genetic strategies to search for mutations in the A. tumefaciens xenognosin recognition system utilized approaches that screened potential mutants for either suppression of virulence and/or vir-gene::lacZ fusion expression Doty et al., 1996; Klee et al., 1983; Stachel et al., 1986), or up-regulation of vir-gene::lacAZ expression in the absence of xenognostic phenols (Chang et al., 1996; Pazour et al., 1991; Ankenbauer et al., 1991; McLean et al., 1994). In these cases, mutations were generated either by transposon mutagenesis, which would completely disrupt the gene and cause polar mutations, or targeted mutagenesis of the expected receptor, virA. In contrast, the strategy provides a selection scheme that can be used to search for mutations affecting phenol-mediated vir gene expression throughout the A. tumefaciens genome. The isolation of mutants that demonstrate a xenognostic phenol specific hypersensitivity demonstrates the power of this strategy and will ultimately facilitate cloning the responsible gene(s).

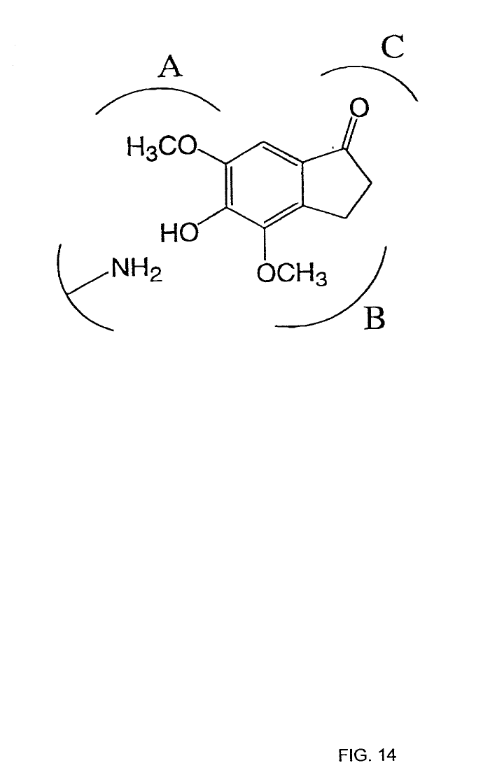

To the extent that the differential responsiveness of the vir gene expression system to the xenognostic phenol can be attributed to differences in receptor affinity, a model for the binding interaction can be developed (FIG. 14). A critical primary amine is placed proximal to the phenolic OH for the proton transfer activation (Lee et al., 1992; Duban et al., 1993; Hess et al., 1991). The ortho memory groups must contribute positively and equally to the binding affinity in the wild-type receptor, but in the mutants, domains A and B cannot be equivalent. In all of the mutants obtained thus far, it is both the interaction between the methoxy group and the domain B that contributes significantly to the hypersensitivity. The observation that the E-indanone, but not AV, is more effective at inducing vir gene expression in the hypersensitive mutants establishes that the alkyl substituent also contributes to the apparent increased binding affinity of the mutants. However, the active (+) trans-CP enantiomer is only slightly more active than wild-type, and the methoxy region of the phenol edge appears to be the most critical.

The phenol hypersensitive mutants are unique in that the novel structural requirements of the phenol for maximal sensitivity are not related to virA or any other gene on the Ti plasmid. To date the only other protein known to be involved in phenol-mediated induction of vir expression is ChvE, a sugar binding protein that interacts with the periplasmic domain of VirA and increases the sensitivity of the system to phenols. Sequence analysis demonstrates that ChvE is wild-type in both sugar dependent ant sugar independent hypersensitive strains. While the hypersensitive phenotype is sugar-independent in many cases, these strains continue to demonstrate the normal response to the inducing sugars.

Previous analyses of VirA have suggested that the linker domain functions as a phenol-mediated activator of the VirA/VirG phosphorylation cascade. Chang & Winans, 1992; Doty et al., 1996; Turk et al., 1994; Chang et al., 1996. This domain is also critical for the transduction of the ChvE-sugar signal that results in increased phenol sensitivity. The results reported here are consistent with a novel model in which the ChvE-sugar interaction with the periplasmic domain of VirA mediates conformational shifts of a hypothetical xenognostic phenol binding protein (Xpb) that is bound to the linker domain of VirA, thereby increasing the apparent affinity of Xpb for the phenols. In the case of the sugar dependent hypersensitive mutants, this conformational shift would be required for mutant Xpbs to exhibit altered affinity. In contrast, the sugar independent mutant Xpbs, while still capable of responding to the ChvE/sugar activation of VirA, can exhibit an increased affinity for the phenols in the absence of the ChvE/sugar activation.

Consistent with this model is the data demonstrating that, as with the non-Ti plasmid-localizet mutations described here, mutations in either the repressing response-regulatory domain or the periplasmic domain of VirA (Chang et al., 1996) can increase sensitivity as well as the structural specificity of the xenognostic phenol. Interestingly, no mutations that affect phenol specificity have been observed in the linker domain of VirA. Taken together, these observations suggest that the linker domain of VirA may serve to integrate signal input, and the activation barrier for signal response can be reduced by mutations in both VirA and the Xpbs. Formally, the Xpb could serve either to activate the phosphotransfer system when phenols are bound or, alternatively, act as a repressor of the phosphotransfer system, in which case repression would be relieved by phenol binding. This model is also consistent with physical evidence for the existence of chromosomally encoded Xbps in Agrobacterium and explains the inability to obtain any physical evidence for xenognoseic phenol-binding to VirA (Lee et al., 1992; Dye & Delmotte, 1997). An alternative model is that the hypersensitive phenotype is the result of increased uptake or altered metabolism of the phenolics in question. While this possibility cannot be rigorously excluded, the current results clearly demonstrate the specificity of the alteration and its importance in both vir gene expression and virulence.

### Example 3

Efficient Expression System for A. tumefaciens

A. Materials and Methods

Bacterial Strains, Plasmas and Oligonucleotides. The bacterial strains & plasmids and oligonucleotides used in this study are listed in the following tables.

TABLE 10

| Strain/plasmid | Relevant characteristics | Reference or source |
|---|---|---|
| E. coli | | |
| BL21 | F⁺ ompT gal [dam] [Iom] hsdSB (r_{B}⁻m_{B}⁻); an E. coli B | Novagen |
| | strain with DE3, a lambda prophage carrying the T7 | |
| | RNA polymerase gene | |
| XL1-Blue | recA1 endA1 gryA96 thi 1 hsdR17 supE44 relA1 lac(F′ | Stratagene |
| | proAB lacl^{r} M15 Tn 10(Tc′)] | |
| A. tumefaciens | | |
| A136 | Strain C58 cured of pTi plasmid, Rif^{r}, Nal^{r} | Watson et al., 1975 |
| A348 | A136 containing pTiA6NC | Garfinkel et at., 1981 |
| A348-3 | virA deletion derivative of A348, Km^{r} | Lee et al., 1992 |
| Plasmids | | |
| Pbluescript II | cloning vector ColE1, Ap^{r} | Stratagene |
| KS (+/−) (pBS) | | |
| PCR2.1 | TA cloning vector, ColE1, Ap^{r} | Invitrogen |
| PET16b | Ap^{r}, T7 promoter, 6xHis tag | Novagen |
| PHP45Ω | source of a 2 Kb Bam HI piece containing the aada* | Prentki et at., 1984 |
| | gene, spec^{r}/Sm^{r} | |
| PJB20 | Broad-host-range plasmid, derivative of pUCD2, IncW, | Beaupre et at., 1997 |
| | pBR322ori, Ap^{r}/Tc^{r} | |
| PMutA | 4.5 kb Kpn I fragment containing P_{virA} -virA(G665D) | McClean et al., 1994 |
| G665D | in pUCD2 | |
| PSM243cd | P_{virB}::lacZ, IncP, Ap^{r} | Stachel et al., 1986 |
| PSW209 | P_{virB}::lacZ fusion derived from pSW243cd, IncP, Km^{r} | S. C. Winans, Cornell |
| | | University |
| PSW209Ω | 2 kb Bam HI fragment from pHP45Ω cloned into | See below |
| | BamHI site of pSW209; P_{virB}::lacZ, IncP, Spec^{r}, Km^{r} | |
| PPC401 | P_{lac}::virG in pTZ18, Ap^{r} | Jin et al., 1990a |
| pQE-30 | P_{N25}-6xHis-MCS-STOP, pBR322ori, Ap^{r}, E. coli | Qiagen |
| | expression vector | |
| pQE-50 | P_{N25}-MCS-STOP, pBR322ori, Ap^{r}, E. coli expression | Qiagen |
| | vector | |
| pUCD2 | Broad-host-range cloning vector; pSa on (IncW), | Close et al., 1984 |
| | pBR322 ori, Tc^{r}, Spec^{r}, Km^{r}, Ap^{r} | |
| pVH1 | AvaI fragment from Kpn I fragment of pVRA8 cloned | See below |
| | into Xho I site of pET16B | |
| pVRA8 | 4.5 kb Kpn I fragment carrying P_{virA} -virA^{wt} from | Lee et al., 1992 |
| | pTiA6 in pUCD2, pBR322ori, IncW, Ap^{r}/Km^{r}/Spec^{r} | |
| pYW10 | pJB20 derivative, with unique Nco I site, Ap^{r} | See below |
| PYW15a | Broad-host-range expression vector, P_{N25}-6xHis-MCS- | See below |
| | STOP, pBR322ori, Inc.W, Ap^{r} | |
| pYW15b | Broad-host-range expression vector, P_{N25}-6xHis-MCS- | See below |
| | STOP, pBR322ori, Inc.W, Ap^{r} | |
| pYW15c | Broad-host-range expression vector, P_{N25}-6xHis-MCS- | See below |
| | STOP, pBR322ori, Inc.W, Ap^{r} | |
| pYW39 | P_{N25}-6xHis-virA (Δ1-284, G665D) in pYW15a, Ap^{r} | See below |
| pYW40 | P_{N25}-virA (Δ1-284, G665D) in pYW15c, Ap^{r} | See below |
| pYW46 | 750 bp Sac I - Kpn I fragment containing virG (second | See below |
| | codon to stop codon of orf) in pBS | |
| pYW47 | P_{N25}-6xHis-virG in pYW15b, Ap^{r} | See below |
| pYW48 | 4.5 Kb Kpn I fragment containing P_{virA}-virA^{wt} cloned | See below |
| | into pYW47, Ap^{r} | |

TABLE 11

| Oligonucleotides | Sequence | |
|---|---|---|
| H3 23mer | 5′-GCC CAT GGC TCG AGA AAT CAT AA -3′ | (Nco I) |
| H4 26mer | 5′-GCC CAT GGA AGC TTG GCC GCG GGT CG-3′ | (Nco I) |
| H5 26mer | 5′-CAG GCC CAT GGC TCG AGA AAT CAT AA-3′ | (Nco I) |
| H6 26mer | 5′-CAG GCC CAT GGT CAG CTA ATT AAG CT-3′ | (Nco I) |
| LKR1 26mer | 5′-GCG GTA CCG A11 GGT TAG CGC GGC GT-3′ | (Kpn I) |
| LKR2 26mer | 5′-GCC CGC GGG CAA C-TC TAC GTC TTG AT-3′ | (Sac II) |
| GS2 26mer | 5′-GC GAG CTC AAA CAC GTT CTT CTT ATC-3′ | (Sac I) |
| GA1 26mer | 5′-GG AAG CTT ATT GGC TCA GGC TGC CAT-3′ | (Hind III) |
| Oligo1 20mer | 5′-TCG AGG AGG CCA TGG TGC GC-3′ | (Nco I) |
| Oligo2 20mer | 5′-AAT TGC GCA CCA TGG CCT CC-3′ | (Nco I) |

Molecular Biological Techniques. PCR products were cloned into the PCR2.1 vector (Invitrogen), and plasmid transformation into E. coli strains employed a heat-shock protocol with competent cells (Stratagene). Plasmid transformation into Agrobacterium was achieved with a Gene Pulser and a 0.2 cm electroporation cuvette (Bio-Rad) at 2.5 KV, 400 ohms, and 25 μF. Electrocompetent cells of Agrobacterium strains were prepared by the method of Cangelosi et al. (1991). Plasmid DNA was isolated by QIAprep spin columns (Qiagen) and plasmid constructs were confirmed by restriction digestion analysis. Restriction enzymes and T4 DNA ligase were obtained from Promega or New England Biolabs.

Analysis of Protein Expression. Luria Broth (LB: Miller, 1972) and Induction Media [IM, per liter 3.g MES hydrate, 50 ml of 20× AB Salts (Cangelosi et al., 1991), 2 ml of 20× AB Buffer (Cangelosi et al., 1991) with 1% glycerol or 1% glucose as carbon source] were used to culture the bacteria. For expression in E. coli, 15 ml pre-warmed LB medium containing appropriate antibiotics was inoculated with an overnight culture of E. coli XL1-Blue (1:50 vol/vol) carrying the various constructs, and grown at 37° C. to an OD₆₀₀ (optical density at 600 nm) ˜0.7-0.9, IPTG was then added to a final concentration of 1 mM, and the induction continued for 5 hours at 37° C. For expression in A. tumefaciens, a 30 ml overnight culture in LB or IM containing appropriate antibiotics was used. Cell lysis of E. coli was achieved by boiling in 2× SDS sample buffer (Novex) and of Agrobacterium by brief sonication on ice. Lysates were clarified by microcentrifugation. For soluble and membrane fractions, 1 liter LB or IM cultures were grown overnight, harvested and lysed as per the native lysis protocol (Qiagen), and fractionated as described in Lee et al. (1992). Lysates and fractions were analyzed by SDS/PAGE conducted in 14% or 8-16% polyacrylamide gels (Novex).

Immunoblot Analysis. Anti-VirA Ab was raised in rabbits using the following strategy. The Mini fragment of pVRA8 carrying virA was digested with Ava I to yield a 453 bp fragment encoding the kinase region of VirA (includes aa 484 to 636 of VirA). This fragment was gel purified using the Gene Clean II kit (Bio 101) and blunt ended using the Klenow fragment of DNA polymerase. pET16b (Novagen) was digested with Xho I and the ends blunted with Klenow. The purified Ava I fragment was ligated to the linearized pET16b with T4 DNA ligase, yielding pVH1. This cloning strategy placed the virA fragment behind both a 6×His tag and the powerful 17 promoter. pVH1 was electroporated into E. coli expression strain, BL21.

BL21/pVH1 was grown and induced according to Novagen instructions. The cells were lysed and the fusion protein purified on a Ni-agarose column according to the QIAexpress (Qiagen) instructions. The purified protein was dialyzed against PBS at 0.8 mg/ml, mixed with Freund's incomplete adjuvant, and injected into rabbits (Cocalico Co., Lancaster, Pa.). The rabbits were boosted at 14, 21 and 35 days, the blood collected by cardiac puncture, and the serum was recovered and stored at −20° C.

For immunoblot analysis, SDS/PAGE was conducted in 14% or 8-16% polyacrylamide gels (Novex), and the proteins were electroblotted (Electro-blotter, Bio-Rad) onto nitrocellulose membranes (Bio-Rad). Blots were probed for VirA and VirA(Δ61-284, G665D) with the polyclonal and VirA Ab whereas the 6×His-tagged VirG protein was probed win anti-RGSHis mAb (Qiagen). Visualization was achieved by using either alkaline phosphatase-conjugated secondary Ab, goat-and-rabbit Ab (Bio-Rad) for anti-VirA and rabbit-anti-mouse Ab (Pierce) for anti-RGSHis, both at 1:1000 dilution followed by 1-Step NBT/BCIP (Pierce) developing, or horseradish peroxidase conjugated anti-mouse secondary Ab (Amersham) with the enhanced chemilluminescence (ECL) Western blot detection system (Amersham).

vir Gene Induction Assay. pSW209 and pSW209Ω were the reporter plasmids used to assay vir gene expression in Agrobacterium, pSW209, which contains lacZ behind thevirB promoter, was linearized with Barn HI and treated with shrimp alkaline phosphatase, pHP45Ω was cut win Barn HI to yield a 2 kb fragment containing aadA, the spectinomycin resistance cartridge. This fragment was ligated to the linearized pSW209 to yield pSW209Ω. For vir expression assays, strains were grown in 30 ml of LB medium supplemented with appropriate antibiotics to OD₆₀₀ 0.4-0.6. The bacteria were harvested by centrifugation at 6000 rpm (GSA rotor) and 4° C. for 15 minutes. After washing with PBS buffer twice, the pellets were stored at −80° C. As needed, cells were thawed diluted to OD₆₀₀, of ˜0.1 into 1 ml of IM with either 1.0% glycerol or 1% glucose and cultured at 20° C. or 28° C. with shaking at 225 rpm. β-galactosidase activity was determined as described by Miller (1972) after a 20 hr induction or at the indicated time intervals.

B Results

Construction of Broad-Host-Range Vectors pYW15a, pYW15b, and pYW15c. The pYW15 series of vectors was constructed so as to create a functional expression system that could be used in bow E. coli and Agrobacterium. First, a unique Nco I restriction site was introduced into pJB20 using two 20-bp oligomers, oligos 1 and 2. Both oligomers contain a Nco I site and a 5′-end complementary to either an Eco RI or Sal I restriction site. The two oligomers were first self-annealed and then ligated into the Eco RI and Sal I sites of pJB20 to give the plasmid pYW10. Therefore, pYW10 contains the newly introduced unique Nco I site with the original Sal I and Eco RI sites destroyed. Second, primers 113 and 114 were used to amplify the coliphage expression elements, less the stop codons following the multiple cloning site, from pQE30 (Qiagen). This was cloned into the Nco I site of pYW10, yielding pYW15a. In addition, primers H5 and H6 were used to amplify pQE30 (Qiagen) and pQE50 (Qiagen) and the expected 190 bp PCR products were cloned into the Nco I site of pYW10 to give pYW15b and pYW15c.

Both the pSa replication origin for plasmid maintenance in Agrobacterium and E. coli strains and the pBR322 replication origin for higher copy number in E. coli (Bolivar et al., 1977) are present. The transcriptional and translational control elements include the coliphage T5 promoter PN25 and two lac operator sequences for controlled protein expression in strains containing the lacl repressor gene, the synthetic ribosome binding site, RBSII, for optimal mRNA recognition and binding, and a multiple cloning site (MCS). In addition, pYW15a and pYW15b express the 6×His affinity tag which facilitates purification of the expressed proteins via the Ni-NTA affinity column (Qiagen). p7W15b and pYW15c also contain translation stop codons downstream of the MCS in all reading frames so as to ensure translational termination of all inserted sequences.

Construction P_{N25}-6×His-virA(Δ-284, G665D) and P_{N25}-virA(Δ1-284, G665D). VirA carrying a G665D mutation gives low constitutive vir gene expression that is further enhanced by AS induction (McLean et al. 1994). The DNA sequence encoding amino acid residues 285-829 of VirA(G665D) was amplified by PCR from the encoding plasmid, pMutA(G665D) with primers LKR1 and LKR2. This ˜1.6 kb PCR product, which carries a deletion of the periplasmic and both transmembrane domains from the wild-type protein, was designated virA(ΔJ284, G665D) and was cloned into PCR2.1 (Invitrogen). The Kpn I fragment containing this entire open reading frame was then released and cloned into the Kpn I site of both pYW15a and pYW15c to give plasmid pYW39 (with) and pYW4O (without an N-terminal 6×His tag), respectively.

Construction of P_{N25}-6×HIS-virG. The DNA sequence encoding VirG (second codon to stop) was PCR amplified front plasmid pPC401 using primers GS2 and GA1. The amplified ˜750 bp PCR product was digested with Sac I and Hind III and cloned into pBS to give pYW46. A Sac I and Kpn I DNA fragment containing virG was released froth pYW46 and cloned into pYW15b to yield pYW47. Finally, the 4.5 Kb Kpn I fragment containing P_{virA}-virA from pVRA8 was cloned into the KpnI site of pYW47 to give pYW48. Therefore, pYW48 contains both a copy of full-size virA^{wt} driven by P_{virA} and a copy of 6×His-virG driven by P_{N25}.

Expression in E. coli and A. tumefaciens. Since the E. coli XL1-Blue strain contains the lac repressor gene lad expression from pYW15 constructs should be regulated in this background. Indeed, major proteins at ˜60 and ˜32 kD appeared with IPTG induction of swains containing pYW39 and pYW48 respectively, each corresponding to the predicted mass of VirA(Δ1-284, G6651) and VirG. However, the majority of these over-expressed proteins in both strains were found to be insoluble (data not shown). The formation of inclusion bodies has complicated previous expression of VirA and VirG in E. coli (Jin et al, 1990a, 1990b), and the inability to solubilize either protein (0.25% Tween-20 and 0.1 mM EGTA) suggested that this limitation had not been solved with over-expression from these plasmids.

In A. tumefaciens, the plasmids were introduced into either A. tumefaciens A348-3/pSW209Ω whose wild-type virA has been deleted by homologous recombination (Lee et al. 1992), or A136/pSW209, a strain lacking the Ti plasmid. Both Be VirA(Δ1-284, G665D) and VirG constructs were constitutively expressed at high levels as confirmed by anti-VirA and anti-ROSHis Westerns. The VirA(Δ1-284, G665D) expressed from either pYW39 or pYW40 localized primarily wide the membrane fraction (data not shown), consistent with observations of truncated VirA constructs driven by the native virA promoter P_{virA}, (Chang et al., 1992). In contrast, the expressed VirG was found in the soluble fraction (data not shown).

Because the swains of Agrobacterium tested here do not produce the lac repressor protein, expression from the P_{N25} promoter is constitutive. This expression in Agrobacterium was not as high as seen in the IPTG induced E. coli background. However, it was much higher than that of the full-sized VirA(G665D) which, because of the G665D mutation is constitutively driven by P_{virA}. While the expression by P_{N25}, in A. tumefaciens has not been compared here directly with that of AS-induced P_{virA}, the expression of VirA by native P_{virA} is induced ˜8-fold with AS induction (Rogowsky et al, 1987; Winans et al., 1988; Turk et al. 1993). Therefore, the coliphage T5 promoter P_{N25} is expected to produce higher amounts of protein in Agrobacterium than the native P_{virA} with AS induction. More significantly, the expression of VirG is no longer dependent on low pH and phosphate, arguing that signal transduction can now be studied independent of the environmental factors that regulate virG expression.

vir Gene Induction: P_{N25}-6×His-VirA(Δ1-284, G665); P_{N25}-VirA(Δ-284, G665D). The VirA(Δ1-284, G665D) protein is constitutively active in Agrobacterium (McLean et al., 1994) but can be further stimulated by AS. Because it is missing the periplasmic domain and both transmembrane regions, VirA(Δ1-284, G665D) cannot respond to the monosaccharides such as glucose, that are known to synergistically enhance vir gene induction by AS (Cangelosi et al., 1990; Shimoda et al., 1990). Therefore, the β-galactosidase assays were conducted in IM with 1% glycerol as a carbon source. The reported temperature-sensitivity of the cytoplasmic domain of VirA (Chang et al., 1991) also necessitated an induction assay temperature of 20° C. rather than 30° C.

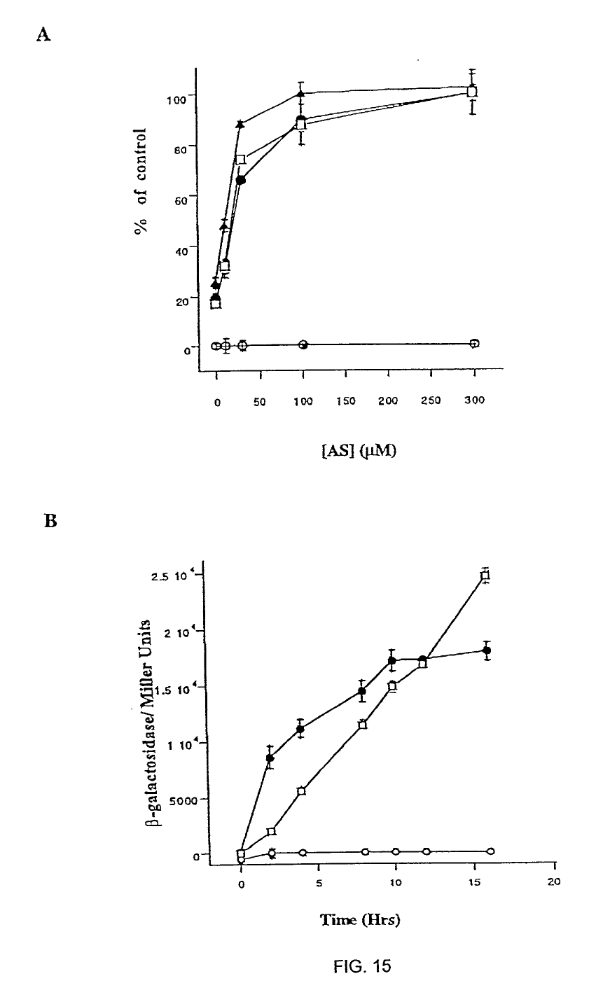

Under these conditions, vir expression by both the P_{N25}-6×His VirA(Δ1-284, G665D) and P_{N25}, virA (Δ1-284, G665D) strains shoved high basal expression, and most importantly, showed a similar sensitivity to AS induction as the full size VirA (G665D) expressed by the native P_{virA} (FIG. 15A). This result is consistent with the expression characteristics seen for the full length VirA(G665D) (McLean et al., 1994), and further demonstrates that a 6×His-tag at the N-terminus of the expressed cytoplasmic domain does not significantly influence functional signal transmission.

vir Gene induction: P_{N25}-6×His-VirG. To evaluate ability of P_{N25} to express functional VirG, pYW48, carrying P_{N25}-6×His-VirG and a wild-type allele of virA, was transferred to the vir reporter strain A136/pSW209. The time dependence of vir gene induction by 100 μM AS is compared in FIG. 15B with that of A348/pSW209, a strain that carries P_{N25}-virG on pTiA6. Very little expression is driven by the native P_{virG} without induction (Winans 1990; Mantis et al., 1992), and consequently a clear lag phase exists at early time points, β-galactosidase levels however rise linearly over at least the first 16 hours due to the autocatalytically upregulated response to AS. In contrast, the strain containing P_{N25}-6×His-VirG accumulates β-galactosidase much faster at early times and reaches a plateau at 10 hours. As with the affinity tagged VirA constructs, the N-terminal 6×His appears to have little influence on the functioning of ViIG in vivo, but this structural modification has not yet been evaluated directly with the wild-type protein.

### Example 4

Backcrossing can be used to improve a starting plant. Backcrossing transfers a specific desirable trait from one source to an inbred or other plant that lacks that trait. This can be accomplished, for example, by first crossing a superior inbred (A) (recurrent parent) to a donor inbred (nonrecurrent parent), which carries the appropriate gene(s) for the trait in question, for example, a construct prepared in accordance with the current invention. The progeny of this cross first are selected in the resultant progeny for the desired trait to be transferred from the non-recurrent parent, then the selected progeny are mated back to the superior recurrent parent (A). After five or more backcross generations with selection for the desired trait, the progeny are hemizygous for loci controlling the characteristic being transferred, but are like the superior parent for most or almost all other genes. The last backcross generation would be selfed to give progeny which are pure breeding for the gene(s) being transferred, i.e. one or more transformation events.

Therefore, through a series a breeding manipulations, a selected transgene may be moved from one line into an entirely different line without the need for further recombinant manipulation. Transgenes are valuable in that they typically behave genetically as any other gene and can be manipulated by breeding techniques in a manner identical to any other corn gene. Therefore, one may produce inbred plants which are true breeding for one or more transgenes. By crossing different inbred plants, one may produce a large number of different hybrids with different combinations of transgenes. In this way, plants may be produced which have the desirable agronomic properties frequently associated with hybrids (“hybrid vigor”), as well as the desirable characteristics imparted by one or more transgene(s).

### Example 5

Marker Assisted Selection

Genetic markers may be used to assist in the introgression of one or more transgenes of the invention from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized.

In the process of marker assisted breeding, DNA sequences are used to follow desirable agronomic traits (Tanksley et al., 1989) in the process of plant breeding. Marker assisted breeding may be undertaken as follows. Seed of plants with the desired trait are planted in soil in the greenhouse or in the field. Leaf tissue is harvested from the plant for preparation of DNA at any point in growth at which approximately one gram of leaf tissue can be removed from the plant without compromising the viability of the plant. Genomic DNA is isolated using a procedure modified from Shure et al. (1983). Approximately one gram of leaf tissue from a seedling is lypholyzed overnight in 15 ml polypropylene tubes. Freeze-dried tissue is ground to a powder in the tube using a glass rod. Powdered tissue is mixed thoroughly with 3 ml extraction buffer (7.0 urea, 0.35 M NaCI, 0.05 M Tris-HCI pH 8.0, 0.01 M EDTA, 1% sarcosine). Tissue/buffer homogenate is extracted with 3 ml phenol/chloroform. The aqueous phase is separated by centrifugation, and precipitated twice using 1/10 volume of 4.4 M ammonium acetate pH 5.2, and an equal volume of isopropanol. The precipitate is washed with 75% ethanol and resuspended in 100-500 μl TE (0.01 M Tris-HCI, 0.001 M EDTA, pH 8.0).

Genomic DNA is then digested with a 3-fold excess of restriction enyzmes, electrophoresed through 0.8% agarose (FMC), and transferred (Southern, 1975) to Nytran (Schleicher and Schuell) using 10× SCP (20× SCP: 2M NaCI, 0.6 M disodium phosphate, 0.02 M disodium EDTA). The filters are prehybridized in 6× SCP, 10% dextran sulfate, 2% sarcosine, and 500 μg/ml denatured salinon sperm DNA and ³²P-labeled probe generated by random priming (Feinberg & Vogelstein, 1983). Hybridized filters are washed in 2× SCP, 1% SDS at 65° for 30 minutes and visualized by autoradiography using Kodak XAR5 film. Genetic polymorphisms which are genetically linked to traits of interest are thereby used to predict the presence or absence of the traits of interest.

Those of skill in the art will recognize that there are many different ways to isolate DNA from plant tissues and that there are many different protocols for Southern hybridization that will produce identical results. Those of skill in the art will recognize that a Southern blot can be stripped of radioactive probe following autoradiography and re-probed with a different probe. In this manner one may identify each of the various transgenes that are present in the plant. Further, one of skill in the art will recognize that any type of genetic marker which is polymorphic at the region(s) of interest may be used for the purpose of identifying the relative presence or absence of a trait, and that such information may be used for marker assisted breeding.

Each lane of a Southern blot represents DNA isolated from one plant. Through the use of multiplicity of gene integration events as probes on the same genomic DNA blot, the integration event composition of each plant may be determined. Correlations may be established between the contributions of particular integration events to the phenotype of the plant. Only those plants that contain a desired combination of integration events may be advanced to maturity and used for pollination. DNA probes corresponding to particular transgene integration events are useful markers during the course of plant breeding to identify and combine particular integration events without having to grow the plants and assay the plants for agronomic performance.

It is expected that one or more restriction enzymes will be used to digest genomic DNA, either singly or in combinations. One of skill in the art will recognize that many different restriction enzymes will be useful and the choice of restriction enzyme will depend on the DNA sequence of the transgene integration event that is used as a probe and the DNA sequences in the genome surrounding the transgene. For a probe, one will want to use DNA or RNA sequences which will hybridize to the DNA used for transformation. One will select a restriction enzyme that produces a DNA fragment following hybridization that is identifiable as the transgene integration event. Thus, particularly useful restriction enzymes will be those which reveal polymorphisms that are genetically linked to specific transgenes or traits of interest.

### Example 6

General Methods for Assays

DNA analysis of transformed plants is performed as follows. Genomic DNA is isolated using a procedure modified from Shure, et al., 1983. Approximately 1 gm callus or leaf tissue is ground to a fine powder in liquid nitrogen using a mortar and pestle. Powdered tissue is mixed thoroughly with 4 ml extraction buffer (7.0 M urea, 0.35 M NaCl, 0.05 M Tris-HCl pH 8.0, 0.01 M EDTA, 1% sarcosine). Tissue/buffer homogenate is extracted with 4 ml phenol/chloroform. The aqueous phase is separated by centrifugation, passed through Miracloth, and precipitated twice using 1/10 volume of 4.4 M ammonium acetate, pH 5.2 and an equal volume of isopropanol. The precipitate is washed with 70% ethanol and resuspended in 200-500 μI TE (0.01 M Tris-HCl, 0.001 M EDTA, pH 8.0).

The presence of a DNA sequence in a transformed cell may be detected through the use of polymerase chain reaction (PCR). Using this technique specific fragments of DNA can be amplified and detected following agarose gel electrophoresis. For example, two hundred to 1000 ng genomic DNA is added to a reaction mix containing 10 mM Tris-HCl pH 8.3, 1.5 mM MgCl₂, 50 mM KCl, 0.1 mg/ml gelatin, 200 μM each dATP, dCTP, dGTP, dTTP, 0.5 μM each forward and reverse DNA primers, 20% glycerol, and 2.5 units Taq DNA polymerase. The reaction is run in a thermal cycling machine as follows: 3 minutes at 94° C., 39 repeats of the cycle 1 minute at 94° C., 1 minute at 50° C., 30 seconds at 72° C., followed by 5 minutes at 72° C. Twenty μl of each reaction mix is run on a 3.5% NuSieve gel in TBE buffer (90 mM Tris-borate, 2 mM EDTA) at 50V for two to four hours.

For Southern blot analysis genomic DNA is digested with a 3-fold excess of restriction enzymes, electrophoresed through 0.8% agarose (FMC), and transferred (Southern, 1975) to Nytran (Schleicher and Schuell) using 10× SCP (20× SCP: 2 M NaCl, 0.6 M disodium phosphate, 0.02 M disodium EDTA). Probes are labeled with ³²P using the random priming method (Boehringer Mannheim) and purified using Quik-Sep® spin columns (Isolab Inc., Akron, Ohio). Filters are prehybridized at 65° C. in 6× SCP, 10% dextran sulfate, 2% sarcosine, and 500 μg/ml heparin (Chomet et al., 1987) for 15 mm. Filters then are hybridized overnight at 65° C. in 6× SCP containing 100 μg/ml denatured salmon sperm DNA and ³²P-labeled probe. Filters are washed in 2× SCP, 1% SDS at 65° C. for 30 mm. and visualized by autoradiography using Kodak XAR5 film. For rehybridization, the filters are boiled for 10 mm. in distilled H₂O to remove the first probe and then prehybridized as described above.

### Example 7

Utilization of Transgenic Crops

The ultimate goal in plant transformation is to produce plants which are useful to man. In this respect, transgenic plants created in accordance with the current invention may be used for virtually any purpose deemed of value to the grower or to the consumer. For example, one may wish to harvest seed from transgenic plants. This seed may in turn be used for a wide variety of purposes. The seed may be sold to farmers for planting in the field or may be directly used as food, either for animals or humans. Alternatively, products may be made from the seed itself. Examples of products which may be made from the seed include, oil, starch, animal or human food, pharmaceuticals, and various industrial products. The food uses of maize, in addition to human consumption of maize kernels, include both products of dry- and wet-milling industries. The principal products of maize dry milling are grits, meal and flour. The maize wet-milling industry can provide maize starch, maize syrups, and dextrose for food use. Maize oil is recovered from maize germ, which is a by-product of both dry- and wet-milling industries.

Maize, including both grain and non-grain portions of the plant, also is used extensively as livestock feed, primarily for beef cattle, dairy cattle, hogs, and poultry. Industrial uses of maize include production of ethanol, maize starch in the wet-milling industry and maize flour in the dry-milling industry. The industrial applications of maize starch and flour are based on functional properties, such as viscosity, film formation, adhesive properties, and ability to suspend particles. The maize starch and flour have application in the paper and textile industries. Other industrial uses include applications in adhesives, building materials, foundry binders, laundry starches, explosives, oil-well muds, and other mining applications. Plant parts other than the grain of maize also are used in industry, for example, stalks and husks are made into paper and wallboard and cobs are used for fuel and to make charcoal. Other means for utilizing plants, such as those that may be made with the current invention, have been well known since the dawn of agriculture and will be known to those of skill in the art in light of the instant disclosure. Specific methods for crop utilization may be found in, for example, Sprague and Dudley (1988), and Watson and Ramstad (1987).

References

The references listed below are incorporated herein by reference to the extent that they supplement, explain, provide a background for, or teach methodology, techniques, and/or compositions employed herein.

Abdullah et al., Biotechnology, 4:1087, 1986.

Abel et al., Science, 232:738-743, 1986.

Bansal, Viret, Haley, Khan, Schantz, Bogorad, “Transient expression from cab-ml and rbcS-m3 promoter sequences is different in mesophyll and bundle sheath cells in maize leaves,” Proc. Nat'l Acad. Sci. USA, 89:3654-3658, 1992.

Barkai-Golan et al., Arch. Microbiol., 116:119-124, 1978.

Battraw and Hall, “Stable transformation of sorghum-bicolor protoplasts with chimeric neomycin phosphotransferase II and beta glucuronidase genes,” Theor. App. Genet., 82(2):161-168, 1991.

Belanger and Kriz, “Molecular basis for allelic polymorphism of the maize globulin-1 gene,” Genet., 129:863-872, 1991.

Bellus, J. Macromol. Sci. Pure Appl. Chem., A31(1):1355-1376, 1994.

Bernal-Lugo and Leopold, Plant Physiol., 98:1207-1210, 1992.

Berzal-Herranz et al., Genes and Devel., 6:129-134, 1992.

Bevan et al., “Structure and transcription of the nopaline synthase gene region of T-DNA,” Nucleic Acids Research, 11(2):369-385, 1983.

Blackman et al., Plant Physiol., 100:225-230, 1992.

Branson and Guss, Proceedings North Central Branch Entomological Society of America, 27:91-95, 1972.

Broakaert et al., Science, 245:1100-1102, 1989.

Buchanan-Wollaston et al., Plant Cell Reports 11:627-631. 1992 .

Campbell (ed.), In: Avermectin and Abamectin, 1989.

Cashmore et al., Gen. Eng. of Plants, Plenum Press, New York, 29-38, 1983.

Cech et al., “In vitro splicing of the ribosomal RNA precursor of Tetrahymena: involvement of a guanosine nucleotide in the excision of the intervening sequence,” Cell, 27:487-496, 1981.

Chandler et al., “Two Regulatory Genes of the Maize Anthocyanin Pathway Are Homologous: Isolation of B Utilizing R Genomic Sequences,” The Plant Cell, 1:1175-1183, 1989.

Chau et al., Science, 244:174-181, 1989.

Chomet et al., EMBO J., 6:295-302, 1987.

Chowrira et al., “In vitro and in vivo comparison of hammerhead, hairpin, and hepatitis delta virus self-processing ribozyme cassettes,” J Biol. Chem., 269:25856-25864, 1994.

Chu et al., Scientia Sinica, 18:659-668, 1975.

Coe et al., In: Corn and Corn Improvement, 81-258, 1988.

Conkling et al., Plant Physiol., 93:1203-1211, 1990.

Cordero, Raventos, San Segundo, “Expression of a maize proteinase inhibitor gene is induced in response to wounding and fungal infection: systemic wound-response of a monocot gene,” Plant J., 6(2)141-150, 1994.

Coxson et al., Biotropica, 24:121-133, 1992.

Cutler et al., J. Plant Physiol., 135:351-354, 1989.

Czapla and Lang, J. Econ. Entomol., 83:2480-2485, 1990.

Davies et al., Plant Physiol., 93:588-595, 1990.

De Block, Botterman, Vandewiele, Dockx, Thoen, Gosselé, Movva, Thompson, Van Mantagu, Leemans, “Engineering herbicide resistance in plants by expression of a detoxifying enzyme,” The EMBO Journal, 6(9):2513-2518, 1987.

De Block, De Brouwer, Tenning, “Transformation of Brassica napus and Brassica oleracea Using Agrobacterium tumefaciens and the Expression of the bar and neo Genes in the Transgenic Plants,” Plant Physiol., 91:694-701, 1989.

Dellaporta et al, In: Chromosome Structure and Function: Impact of New Concepts, 18th Stadler Genetics Symposium, 11:263-282, 1988.

Dennis, Gerlach, Pryor, Bennetzen, Inglis, L1ewellyn, Sachs, Ferl, Peacock, “Molecular analysis of the alcohol dehydrogenase (Adh1) gene of maize,” Nucl. Acids Res., 12(9):3983-4000, 1984.

Depicker et al., Plant Cell Reports, 7:63-66, 1988.

Dure et al., Plant Molecular Biology, 12:475-486, 1989.

Ebert et al., 84:5745-5749, Proc. Nat'l Acad. Sci. USA, 1987

Erdmann et al., Mol. Jour. Gen. Micro., 138:363-368, 1992.

Feinberg and Vogelstein, Anal. Biochem., 132:6-13, 1983.

Finkle et al., Plant Sci., 42:133-140, 1985.

Fitzpatrick, Gen. Engineering News, 22:7, 1993.

Forster and Symons, “Self-cleavage of plus and minus RNAs of a virusoid and a structural model for the active sites,” Cell, 49:211-220, 1987.

Fraley et al., Bio/Technology, 3:629-635, 1985.

Franken, Niesbach-Klosgen, Weydemann, Marechal-Drouard, Saedler, Wienand, “The duplicated chalcone synthase genes C2 and Whp (white pollen) of Zea mays are independently regulated; evidence for translational control of Whp expression by the anthocyanin gene” EMBO J., 10(9):2605-2612, 1991.

Fransz, de Ruijter, Schel, “Isoenzymes as Biochemical and Cytochemical Markers in Embryogenic Callus of Maize (Zea mays L.),” Plant Cell Reports, 8:67-70, 1989.

Fromm et al., Nature, 312:791-793, 1986.

Fromm et al., The Plant Cell, 1:977-984, 1989.

Gatehouse et al., J. Sci. Food. Agric., 35:373-380, 1984.

Gelvin et al., In: Plant Molecular Biology Manual, 1990.

Gerlach et al., “Construction of a plant disease resistance gene from the satellite RNA of tobacco ringspot virus,” Nature (London), 328:802-805, 1987.

Ghosh-Biswas, Iglesias, Datta, Potrykus, “Transgenic Indica rice (Oryza sativa L.) plants obtained by direct gene transfer to protoplasts,” J. Biotechnol., 32(1):1-10, 1994.

Goring et al., Proc. Nat'l Acad. Sci. USA, 88:1770-1774, 1991.

Guerrero et al., Plant Molecular Biology, 15:11-26, 1990.

Gupta et al., Proc. Nat'l Acad. Sci. USA, 90:1629-1633, 1993.

Hamilton et al., Proc. Nat'l Acad. Sci. USA, 93(18):9975-9979, 1996.

Hammock et al., Nature, 344:458-461, 1990.

Haseloff and Gerlach, “Simple RNA enzymes with new and highly specific endoribonuclease activities,” Nature, 334:585-591, 1988.

Haseloff et al., “Removal of a cryptic intron and subcellular localization of green fluorescent protein are required to mark transgenic Arabidopsis plants brightly,” Proc Natl Acad Sci USA, 18;94(6):2122-2127, 1997.

Hemenway et al., The EMBO J., 7:1273-1280, 1988.

Hiei et al., “Transformation of rice mediated by Agrobacterium tumefaciens,” Plant. Mol. Biol., 35(1-2):205-218, 1997.

Hilder et al., Nature, 330:160-163, 1987.

Hinchee et al., Bio/technol., 6:915-922, 1988.

Hudspeth and Grula, Plant Mol. Biol., 12:579-589, 1989.

Ikeda et al., J. Bacteriol., 169:5615-5621, 1987.

Ikuta et al., Bio/technol., 8:241-242, 1990.

Johnson et al., Proc. Nat'l Acad. Sci. USA, 86:9871-9875, 1989.

Joshi, Nucleic Acids Res., 15:6643-6653, 1987.

Joyce, “RNA evolution and the origins of life,” Nature, 338:217-244, 1989.

Kaasen et al., J. Bacteriology, 174:889-898, 1992.

Kaeppler et al., Plant Cell Reports 9: 415-418, 1990.

Kaeppler, Somers, Rines, Cockburn, “Silicon carbide fiber-mediated stable transformation of plant cells,” Theor. Appl. Genet. 84(5-6):560-566, 1992.

Karsten et al., Botanica Marina, 35:11-19, 1992.

Katz et al., J Gen. Microbiol., 129:2703-2714, 1983.

Keller et al., EMBO J, 8(5):1309-1314, 1989.

Kim and Cech, “Three dimensional model of the active site of the self-splicing rRNA precursor of Tetrahymena,” Proc. Nat'l Acad. Sci. USA, 84:8788-8792, 1987.

Klee, Yanofsky, Nester, “Vectors for transformation of higher plants,” Bio-Technology, 3(7):637-642, 1985.

Kohler, Liaud, Mendel, Cerff, Hehl, “The maize GapC4 promoter confers anaerobic reporter gene expression and shows homology to the maize anthocyanin regulatory locus C1,” Plant Mol. Biol., 29(6):1293-1298, 1995.

Koster and Leopold, Plant Physiol., 88:829-832, 1988.

Kriz, Boston, Larkins, “Structural and transcriptional analysis of DNA sequences flanking genes that encode 19 kilodalton zeins,” Mol. Gen. Genet., 207(1):90-98, 1987.

Kunkel, T. A. et al., “Rapid and efficient site-specific mutagenesis without phenotypic selection,” Methods Enzymol, 154:367-382, 1987.

Langridge and Feix, “A zein gene of maize is transcribed from two widely separated promoter regions,” Cell, 34:1015-1022, 1983.

Langridge et al., Proc. Nat'l Acad. Sci. USA, 86:3219-3223, 1989.

Laufs et al., Proc. Nat'l Acad. Sci., 7752-7756, 1990.

Lawton et al., Plant Mol. Biol. 9:315-324, 1987.

Lee; Suh; Lee, “Gene transfer into intact cells of tobacco by electroporation,” Korean J Genet, 11(2):65-72, 1989.

Levings, Science, 250:942-947, 1990.

Lieber and Strauss, “Selection of efficient cleavage sites in target RNAs by using a ribozyme expression library.” Mol. Cell. Biol., 15: 540-551, 1995.

Lindstrom et al., Developmental Genetics, 11:160, 1990.

Loomis et al., J. Expt. Zoology, 252:9-15, 1989.

Ma et al., Nature, 334:631-633, 1988.

Mariani et al., Nature, 347:737-741, 1990.

Martinez, Martin, Cerff, “Structure, evolution and anaerobic regulation of a nuclear gene encoding cytosolic glyceraldehyde-3-phosphate dehydrogenase from maize,” J. Mol. Biol., 208(4):551-565, 1989.

McCormac et al., “The use of visual marker genes as cell-specific reporters of Agrobacterium—mediated T-DNA delivery to wheat (Triticum aestivum L.) and barley (Hordeum vulgare L.),” Euphytica, v.99 (1):17-25, 1998.

Michel and Westhof, “Modeling of the three-dimensional architecture of group I catalytic introns based on comparative sequence analysis,” J. Mol. Biol., 216:585-610, 1990.

Mundy and Chua, The EMBO J., 7:2279-2286, 1988.

Murakami, Anzai, Imai, Satoh, Nagaoka, Thompson, “The bialaphos biosynthetic genes of Streptomyces hygroscopicus: Molecular cloning and characterization of the gene cluster,” Mol. Gen. Genet., 205:42-50, 1986.

Murashige and Skoog, Physiol. Plant., 15:473-497, 1962.

Murdock et al., Phytochemistry, 29:85-89, 1990.

Nagatani, Honda, Shimada, Kobayashi, “DNA delivery into rice cells and transformation using silicon carbide whiskers,” Biotech. Tech., 11(7):471-473, 1997.

Napoli, Lemieux, Jorgensen, “Introduction of a chimeric chalcone synthase gene into petunia results in reversible co-suppression of homologous genes in trans,” Plant Cell, 2:279-289, 1990.

Odell et al., “Identification of DNA sequences required for activity of the cauliflower mosaic virus 35S promoter,” Nature, 313:810-812, 1985.

Ogawa et al., Sci. Rep., 13:42-48, 1973.

Omirulleh, Abraham, Golovkin, Stefanov, Karabaev, Mustardy, Morocz, Dudits, “Activity of a chimeric promoter with the doubled CaMV 35S enhancer element in protoplast-derived cells and transgenic plants in maize,” Plant Mol. Biol., 21 (3):4 15-428, 1993.

Ow et al., Science, 234:856-859, 1986.

Palukaitis et al., “Characterization of a viroid associated with avacado sunblotch disease,” Virology, 99:145-151, 1979.

Perlak et al., Proc. Nat'l Acad. Sci. USA, 88:3324-3328, 1991.

Perriman et al., “Extended target-site specificity for a hammerhead ribozyme,” Gene, 113:157-163, 1992.

Phi-Van et al., Mol. Cell. Biol., 10:2302-2307, 1990.

Piatkowski et al., Plant Physiol., 94:1682-1688, 1990.

Poszkowski et al., EMBO J., 3:2719, 1989.

Potrykus et al., Mol. Gen. Genet., 199:183-188, 1985.

Poulsen et al., “Characterization of an RBC-S gene from Nicotiana plumbaginfolia and expression of an RBC-S-CAT chimeric gene in homologous and heterologous nuclear background,” Mol. Gen. Genet., 205(2):193-200, 1986.

Prasher et al., Biochem. Biophys. Res. Commun., 126(3):1259-1268, 1985.

Prody et al., “Autolytic processing of dimeric plant virus satellite RNA.” Science, 231:1577-1580, 1986.

Quigley, Brinkman, Martin, Cerff, “Strong functional GC pressure in a light-regulated maize gene encoding subunit GAPA of chloroplast glyceraldehyde-3-phosphate dehydrogenase: implications for the evolution of GAPA pseudogenes,” J. Mol. Evol., 29(5):412-421, 1989.

Ralston, English, Dooner, “Sequence of three bronze alleles of maize and correlation with the genetic fine structure,” Genet., 119(l):185-197, 1988.

Reed et al., J. Gen. Microbiology, 130:1-4, 1984.

Reichel et al., Proc. Nat'l Acad. Sci. USA, 93(12) p.5888-5893. 1996.

Reina, Ponte, Guillen, Boronat, Palau, “Sequence analysis of a genomic clone encoding a Zc2 protein from Zea mays W64 A,” Nucl. Acids Res., 18(21):6426, 1990.

Reinhold-Hurek and Shub, “Self-splicing introns in tRNA genes of widely divergent bacteria,” Nature, 357:173-176, 1992.

Rensburg et al., J. Plant Physiol., 141:188-194, 1993.

Rochester, Winer, Shah, “The structure and expression of maize genes encoding the major heat shock protein, hsp70,” EMBO J., 5:451-458, 1986.

Rogers et al., Methods Enzymol., 153:253-277, 1987.

Sambrook, Fritsch, and Maniatis, In Molecular Cloning: A Laboratory Manual, Second edition, Cold Spring Harbor Laboratory, Cold Spring Harbor, N.Y., 1989.

Schwob et al., “Molecular analysis of three maize 22 kDa auxin-binding protein genes-transient promoter expression and regulatory regions,” Plant J 4(3):423-432, 1993.

Shagan and Bar-Zvi, Plant Physiol., 101:1397-1398, 1993.

Shapiro, In: Mobile Genetic Elements, 1983.

Sheen et al., Plant Journal, 8(5):777-784, 1995.

Shure et al., Cell, 35:225-233, 1983.

Simpson, Science, 233:34, 1986.

Singsit, Adang, Lynch, Anderson, Wang, Cardineau, Ozias-Akins, “Expression of a Bacillus thuringiensis cryIA(c) gene in transgenic peanut plants and its efficacy against lesser cornstalk borer,” Transgenic Res., 6(2):169-176, 1997.

Skriver and Mundy, Plant Cell, 2:503-512, 1990.

Smith, Watson, Bird, Ray, Schuch, Grierson, “Expression of a truncated tomato polygalacturonase gene inhibits expression of the endogenous gene in transgenic plants,” Mol. Gen. Genet., 224:447-481, 1990.

Southern, “Detection of specific sequences among DNA fragments separated by gel electrophoresis,” J Mol. Biol., 98:503-517, 1975.

Spencer et al., “Segregation of transgenes in maize,” Plant Molecular Biology, 18:201-210, 1992.

Sprague and Dudley, eds., Corn and Improvement, 3rd ed., 1988.

Stalker et al., Science, 242:419-422, 1988.

Stief et al, Nature 341:343 1989.

Stougaard, J, The Plant Journal, 3:755-761, 1993.

Sullivan, Christensen, Quail, “Isolation and characterization of a maize chlorophyll a/b binding protein gene that produces high levels of mRNA in the dark,” Mol. Gen. Genet., 215(3):431-440, 1989.

Sutcliffe, “Nucleotide sequence of the ampicillin resistance gene of Escherichia coli plasmid pBR322,” Proc. Nat'l Acad. Sci. USA, 75:3737-3741, 1978.

Symons, “Avacado sunblotch viroid: primary sequence and proposed secondary structure.” Nucl. Acids Res., 9:6527-6537, 1981.

Symons, “Small catalytic RNAs.” Annu. Rev. Biochem., 61:641-671, 1992.

Tanksley et al., Bio/Technology, 7:257-264, 1989.

Tarczynski et al., “Expression of a bacterial mtlD gene in transgenic tobacco leads to production and accumulation of mannitol,” Proc. Nat'l Acad. Sci. USA, 89:1-5, 1992.

Tarczynski et al., “Stress Protection of Transgenic Tobacco by Production of the Osmolyte Mannitol,” Science, 259:508-510, 1993.

Thompson et al., “Characterization of the herbicide-resistance gene bar from Streptomyces hygroscopicus,” The EMBO Journal, 6(9):2519-2523, 1987.

Thompson, Drayton, Frame, Wang, Dunwell, “Maize transformation utilizing silicon carbide whiskers: A review” Euphytica, 85(1-3):75-80, 1995.

Tian, Sequin, Charest, “Expression of the green fluorescent protein gene in conifer tissues,” Plant Cell Rep., 16:267-271, 1997.

Tingay et al., The Plant Journal v.11(6) p.1369-1376. 1997.

Tomic et al., Nucl. Acids Res., 12:1656, 1990.

Toriyama et al., Theor Appl. Genet., 73:16, 1986.

Twell et al., Plant Physiol 91:1270-1274, 1989.

Ugaki et al., Nucl. Acid Res., 19:371-377, 1991.

Upender, Raj, Weir, “Megaprimer method for in vitro mutagensis using parallel templates,” Biotechniques, 18(1):29-30, 1995.

Van der Krol, Mur, Beld, Mol, Stuitje, “Flavonoid genes in petunia: addition of a limiting number of copies may lead to a suppression of gene expression,” Plant Cell, 2:291-99, 1990.

Van Tunen et al., EMBO J., 7:1257, 1988.

Vasil et al., Plant Physiol., 91:1575-1579, 1989.

Vernon and Bohnert, The EMBO J, 11:2077-2085, 1992.

Vodkin et al., Cell, 34:1023, 1983.

Vogel, Dawe, Freeling, “Regulation of the cell type-specific expression of maize Adhl and Shl electroporation-directed gene transfer into protoplasts of several maize tissues,” J. Cell. Biochem., (Suppl. 0) 13:Part D, 1989.

Walker et al., Proc. Nat'l Acad. Sci. USA, 84:6624-6628, 1987.

Wandelt and Feix, “Sequence of a 21 kd zein gene from maize containing an in-frame stop codon,” Nucl. Acids Res., 17(6):2354, 1989.

Wang et al., “Characterization of cis-Acting Elements Regulating Transcription from the Promoter of a Constitutively Active Rice Actin Gene,” Molecular and Cellular Biology, 12(8):3399-3406, 1992.

Watrud et al., In: Engineered Organisms and the Environment, 1985.

Watson and Ramstad, eds., Corn: Chemistry and Technology, 1987.

Wenzler et al., Plant Mol. Biol., 12:41-50, 1989.

Withers and King, Plant Physiol., 64:675-678, 1979.

Wolter et al., The EMBO J., 4685-4692, 1992.

Xiang and Guerra, Plant Physiol., 102:287-293, 1993.

Xu et al., Plant Physiol., 110:249-257, 1996.

Yamada et al., Plant Cell Rep., 4:85, 1986.

Yamaguchi-Shinozaki et al., Plant Cell Physiol., 33:217-224, 1992.

Yang and Russell, Proc. Nat'l Acad. Sci. USA, 87:4144-4148, 1990.

Yuan and Altman, “Selection of guide sequences that direct efficient cleavage of mRNA by human ribonuclease P,” Science, 263:1269-1273, 1994.

Yuan et al., “Targeted cleavage of mRNA by human RNase P,” Proc. Nat'l Acad. Sci. USA, 89:8006-8010, 1992.

Zhang et al., “Agrobacterium-mediated transformation of elite indica and japonica rice cultivars,” Mol Biotechnol, 8(3):223-231, 1997.

Zheng and Edwards, “Expression of resistance to barley stripe mosaic virus in barley and oat protoplasts, J. Gen. Virol., 71:1865-1868, 1990.

Zukowsky et al., Proc. Nat'l Acad. Sci. USA, 80:1101-1105, 1983.

Ausubel, et al., (ed.), 1991, In: Current protocols in molecular biology; Greene Publishing Associates and Wiley-Interscience. New York.

Bagdasan et al., 1983. Gene. 26, 273-282.

Bujard, et al., 1987, Methods in Enzymology, 155, 416-33.5

Beaupre, et al., J Bacteriol, 179:78-89, 1997.

Bolivar F, et al., Gene. 2, 95-113, 1977.

Cangelosi, et al., Proc. Nat'l. Aced. Sci. USA 81, 708-6712, 1990.

Cangelosi, et al., 1991. Methods in Enzymology Vol. 204, 384-397.

Chang and Winans, 1992, J. Bacterial. 174, 7033-7039.

Chen and Winans, 1990., J. Bacterial. 173, 1139-1144.

Close, et al. 1984, Plasmid 12, 111-118.

Garfinkel, et al., 1981. Cell 27, 143-53.

Gentz, and Bujard, 1985, J. Bacterial. 164, 70-77.

Hess, et al., 1991. Proc. IVatl. Acad. Sci. USA 88, 7854-7858.

Jin, et al., 1990a, J. Bacteriol. 172, 4945-4950.

Jin, et al., 1990b. J. Bacteriol 172, 525-530.

Jin. et al., 1987. J Bacteriol, 169, 4417-4425.

Lee, et al., 1992., Proc. Natl. Acad. Sci. USA 89, 8666-8670.

Liu and Gelvin, 1992, Plant Mol Biol, 20, 1071-1087.

Lohrke et al., 1999., J. Bacteriol., 181, 4533-4539.

Mantis and Winans, 1992., J Bacteriol, 174, 1189-1196.

McLean, et al., 1994. Journal of Biological Chemistry 269. 2645-2651

Miller, I. H., 1992. Experiments in molecular genetics, Cold Springs Harbor Laboratory, Cold Springs Harbor, N.Y.

Newman and Fuqua, 1999. Gene 227, 197-203.

Prentki and Mitch. 1984. Gene 29, 303-313.

Rogowsky, et al., 1987. J. Bacterial. 169, 5101-5112.

Shimoda. et al., 1990. Proc, Natl. Acad. Sci. USA 87, 6684-6688.

Stachel, et al., 1985. LIMBO J. 4, 891-898

Stachel and Zarnbrsrski, 1986. Cell 46, 325-333.

Stuart, et al., 1987. Gene 55, 95-103.

Turk, et al., 1993. Mol. Microbial. 7, 719-724.

Vernade, et al., 1988. J. Bacteriol, 170, 5822-5829.

Watson et al., 1975. J. Bacteriol, 123, 255-64.

Winans, et al., 1988. J. Bacteriol. 110, 4047-4054.

Winans, S. C. 1990. J. Bactenol 172, 2433-2438.

Binns, Thomahow, Annu. Rev. Microbiol., 1998, 42:575. (b) Winans, Microbiol. Rev. 1992, 56, 12.

Zambryski, P. C. Annu. Rev. Plant Physiol. Plant Mol. Biol. 1992, 43,465.

Duban, M. E.; Lee, K.; Lynn, D. G. Mol. Microbiol. 1993, 7, 637.

Heath J. D.; Charleds, T. C.; Nester E. W. In Two-Component Signal Transduction; Hoch, J. A. Shihavy, T. J. Ed.; American Society for Microbiology, Washington, D.C., 1995.

Chilton, M. D. Pro. Nat'l Acad. Sci. USA 1993, 90, 3119.

Raineri, D. M.; Boulton, M. I.; Davies, J. W.; Nester, E. W. Proc. Nat'l Acad. Sci. USA 1993, 90, 3549.

Hansen, G.; Das, A.; Chilton, M. D. Proc. Nat'l Acad. Sci. USA 1994, 91, 7603.

Heth, J. D.; Boulton, M. I.; Raineri, D. M.; Doty, S. L.; Mushegian, A. R.; Charles, T. C.; Davies, J. W.; Nester, E. W. MPMI 1997, 10, 221.

Shen, W. H.; Escudero, J.; Schläppi, M.; Ramos, C.; Horn, B.; Koukoliková-Nicola, Z. (1993) Proc. Nat'l Acad., Sci. USA 1993, 90, 1488.

Sahi, S. V.; Chilton, M. -D.; Chilton, W. S. Proc. Nat'l Acad. Sci. USA 1990, 87, 3879.

Hess, K M.; Dudley. M. W.; Lynn, D. G.; Joerger, R. D.; Binns, A. N. Proc. Nat'l Acad. Sci. USA 1991, 88, 7854.

Lee, K.; Dudley, M. W.; Hess; K. M.; Lynn, D. G.; Joerger, R. D.; Binns, A. N. Proc. Nat'l Acad. Sci. USA 1992, 89, 8666.

McLean, B. G.; Greene, E. A.; Zambryski, P. C. Biochem. 1994, 269, 2645.

Chang, C. -H.; Winans, S. C. J. Bacteriol. 1992,174, 7033.

Hofman, J.; Hofmanova, O.; Hanus, V. (1970) Tet. Lett. 1970, 3213-3214.

Gambrow, H. J.; Luckge, J.; Klausener, A.; Muller, E. Z. Natuforsch, 1986, 41C, 684.

Herdin, P. A.; Davis, F. M.; Williams, W. P. J. Chem. Ecol., 1993, 19, 531.

Chang. M.; Netzly, D. H.; Butler, L. G.; Lynn, D. G. J. Am. Chem. Soc. 1986,108, 7858.

Fate, G. D.; Lynn, D. G. J. Am. Chem. Soc. 1996, 118, 11369.

Fate, G.; Chang, M.; Lynn, D. G. Plant Physiol. 1990, 93, 201.

Zupan and Zambryski, Plant Physical, 107:1041-1041, 1995.

Hooykaas and Beijersbergen, Phytopathol, 32:157-179, 1994.

Zupan and Zambryski, Plant Physical, 107:1041-1041, 1995.

Hooykaas and Beijersbergen, Phytopathol, 32:157-179, 1994.

Christie, J. Bateriol., 179:3085-3094, 1997.

Birch, Annu. Rev. Plant Phys., 48:297-326, 1997.

Winans, Microbial. Rev., 56:12-31, 1992.

Pirrung,. Chem & Biol., 6:R167-R175, 1999.

McEvoy and Dahlquist, Curr. Opin. Struc. Biol., 7:193-797, 1997.

Heath et al., In: J. A. Hoch and T. J. Silhavy (eds.), Two-Component Signal Transduction, ASM Press, Washington, D.C., pp 367-385, 1995.

Swanson et al., Trends Biochem. Sci., 19:485-490, 1994.

Parkinson and Kofoid, Ann. Rev. Genet, 26:71-112, 1992.

Parkinson, Cell, 73:857-871, 1999.

Melchers et al., EMBO J. 8:1919-1925, 1989.

Cangelosi et al., Proc. Natl. Acad. Sci. USA, 87:6708-6712, 1990.

Shimoda et al., Proc. Natl. Acad. Sci. USA, 87:6684-6688, 1990.

Banta et al., J. Bacteriol. 176:3242-3249, 1994.

Turk et al., J. Bacteriol., 175:570 5709, 1993.

Machida et, Nester and Verma (eds.), In: Advances in molecular genetics of plant-microbe Interactions, Kluwer Academic Publishers, The Netherlands, pp 85-96, 1993.

Chang, J. Bacteriol., 174:70337039, 1992.

Doty et al., J. Bacteriol., 178:961-970, 1996.

Turk et al., Plant Molecular Biology. 25:899-907, 1994.

Chang et al., J. Bacteriol., 8:4710 4716, 1996.

Lee et al., Proc. Natl. Acad. Sci. USA, 92:12245-12249, 1995.

Peng et al., J. Bacteriol., 180:5632-5638, 1998.

Lee et al., Proc. Natl. Acad. Sci. USA, 89:8666-8670, 1992.

Dye & Delmotte (1997). Rfochem. J.321, 319-334 Part 2.

Duban et al., (1993). Mol. Microbial. 7, 637-645.

Melcher et al., (1989). Mol. Microbiol. 3, 969-977.

Spencer & Towers (1988). Phytochemistr. 27, 2781-2785.

Teutonico (1991) Plant Physiol, 97, 288-297.

On et al., (1992). Plant Physiol. 98, 343-352.

Lynn et al., (1987). Proc NatL Acad. Sci USA. 84, 615-619.

Binns et al., (1987). Proc. Natl. Acad Sci. USA 84. 980-984.

Tamagnone et al., (1998). Plant Cell 10, 1801-1816.

Dudley, M. (1991). Ph.D. Diss. Synthetic probes of signal transduction pathways. The University of Chicago.

Hirai et al., (1994). BioSci. Biotcch Biochem. 58, 1679-1684.

Aratani, T. (1985). Pure and Appl. Chem. 57, 1839-1844.

Noyori, R. (1990). Science 248, 1194-1199.

Salomon & Kochi. (1973). J. Am. Chem. Soc. 95, 3300-3310.

Lownthal & Masatnune (1990). Tet. Lett, 31, 6005-6008.

Lownthal & Masunune (1991). Tet. Lett. 32, 7373-7376.

Evans, et al., (1991) J. Am. Chem. Soc. 113,726-728.

Evams et al., (1992) Angew. Chem. Int. Ed Eng. 31, 430-432.

Harada, & Nakanishi, K. (1972). Accts. of Chem. Research 8, 257-263.

Harada, & Nakanishi, (1983). Circular dichroic spectroscopy-excitor coupling in organic stereochemistry. University Sciencc Books, Mill Valley.

Charette, & Jutean, H. (1994). J. Am. Chem. Soc. 116, 2651-2652.

Charette et al., (1995). J. Org. Chem 60, 1081-1083.

Theberge et al., (1996) J. Org. Chem. 61, 8792-8798.

Simmons, H. E & Smith, R. D. (1958). J. Am. Chem. Soc. 80. 5323-5324.

Simmons, H. E. & Smith, R. D. (1959). J. Am. Chem. Soc. 81, 4256-4264.

Furukawa et al., (1961). Tetrahedron Lett. 3353-3354.

Kurukawa et al., (1968). Tetrahedron, 24, 53-58.

Ward et al., (1990). J. Bacteriol. 172, 5187-5199.

Klee, et al., (1983). J. Bacteriol 153, 878-883.

Stachel, S. E. & Nester, El. W. (1986). EMBO J. 5, 1445-1454.

Pazour, et al., (1991). Proc. Natl. Acad. Sci. USA 88, 6941-6945.

Ankenbauer, et al., (1991). Molecular Plant-Microbe Interactions 4, 400-406.

McLean et al., (1994). J. Biol. Chem. 269, 2645-2651.

Hess, et al., (1991). Proc. Natl. Acad. Sci. USA 88, 785˜58.

Gong, et al., (1998). Proc. Natl Acad Sci. USA95. 15177-15182.

Shimoda, et al., (1993). 3. Biol. Chem. 268, 26552-26558

Wanner, B. L. (1993). J. Cell. Biochem. 51, 47-53.

Shi et al., (1999). Biochemistry 38, 10119-10125.

Garfinkel et al., (1981). Cell 27. 143-153.

Farrand etal., (1989). J. Bacteriol. 171, 5314-5321.

Winans, et al., (1988). J. Bacterial. 170, 4047-4054.

Fromm, et al., (1986). Nature, 319, 791-793.

Beck, et al., (1982). Gene 19, 327-336.

Fuqua & Winans. (1994). J. Bacterial. 176, 2796-2806.

Ma et al., (1992). Gene 117, 161-167.

Miller, J. H. (1972). Experiments in molecular genetics, Cold Spring Harbor Laboratory, Cold Spring Harber, N.Y.

Beaupre, et al., (1997). J. Bacteriol. 179, 18-89

Bohne, et al., (1998). Proc. Natl Acad Sci. USA. 95, 7057-7062.

Effenberger & Gutmann. (1982). Chem. Ber. 115, 1089-1102.

What is claimed is:

1. A method for selecting a MDIBOA/DIMBOA resistant Agrobacterium strain comprising:
(a) providing an Agrobacterium cell;
(b) culturing said Agrobacterium cell with a phenolic inducer of the vir pathway and under other conditions supporting Agrobacterium replication, but including MDIBOA/DIMBOA at concentrations sufficient to inhibit Agrobacterium replication; and
(c) isolating an Agrobacterium cell that has replicated in the culture of step (b),
thereby selecting MDIBOA/DIMBOA a resistant Agrobacterium strain.

2. The method of claim 1, wherein the Agrobacterium cell of step (a) further comprises an antibiotic resistance gene under the control of a promoter that is upregulated by a vir gene product, and the culture conditions of step (b) include the antibiotic, resistance to which is provided by the antibiotic resistance gene, at concentrations sufficient to inhibit Agrobacterium replication.

3. The method of claim 2, wherein said antibiotic resistance gene is kan^{r}, and said antibiotic is kanamycin.

4. The method of claim 3, wherein kanamycin is present at about 40-50 μM.

5. The method of claim 2, wherein DIMBOA is present at about 50-100 μM.

6. The method of claim 2, wherein phenolic induction uses acetosyringone.

7. The method of claim 6, wherein acetosyringone is present at about 30-100 μM.

8. The method of claim 2, further comprising culturing in the presence of a sugar.

9. The method of claim 8, wherein said sugar is glucose, and the concentration is 0.1%-1%.

10. The method of claim 2, further comprising:
(d) making a replicate culture of the selected Agrobacterium cell; and
(e) culturing the selected Agrobacterium cell in the presence of MDIBOA/DIMBOA and antibiotic, but without said phenolic inducer,
wherein a cell that replicates in step (e) is identified as having a constitutively activated vir pathway, and a cell that does not replicate in step (e) is identified as having a vir-mediated MDIBOA/DIMBO resistant mutation.

11. The method of claim 2, wherein the Agrobacterium of step (a) is A348/pAC2.

12. The method of claim 1, further comprising treating a replicate culture of the Agrobacterium cell of step (a) under conditions supporting Agrobacterium replication, but excluding said phenolic inducer and including MDIBOA/DIMBOA at concentrations sufficient to inhibit Agrobacterium replication.

13. A method for selecting a phenol hypersensitive Agrobacterium strain comprising:
(a) providing an Agrobacterium cell that comprises an antibiotic resistance gene under the control of a promoter that is upregulated by a vir gene product;
(b) culturing said Agrobacterium cell with varying levels of a phenolic inducer and under other conditions supporting Agrobacterium replication, but including antibiotic resistance to which is provided by the antibiotic resistance gene, at concentrations sufficient to inhibit Agrobacterium replication; and
(c) isolating an Agrobacterium cell receiving the lowest level of phenolic induction that has replicated in the culture of step (b),
thereby selecting a first phenol hypersensitive Agrobacterium strain.

14. The method of claim 13, further comprising repeating the steps (a)-(c) with said first phenol hypersensitive Agrobacterium cell, thereby obtaining a second phenol hypersensitive Agrobacterium cell.

15. The method of claim 13, wherein said antibiotic resistance gene is kan^{r}, and said antibiotic is kanamycin.

16. The method of claim 15, wherein kanamycin is present at about 40-50 μM.

17. The method of claim 13, wherein phenolic induction uses acetosyringone.

18. The method of claim 17, wherein acetosyringone is present at about 10 μM.

19. The method of claim 13, further comprising culturing in the presence of a sugar.

20. The method of claim 19, wherein said sugar is arabinose, and the concentration is 0.1-1%.

21. A MDIBOA/DIMBOA resistant Agrobacterium strain selected according to the method comprising:
(d) providing an Agrobacterium cell;
(e) culturing said Agrobacterium cell with a phenolic inducer and under other conditions supporting Agrobacterium replication, but including
MDIBOA/DIMBOA at concentrations sufficient to inhibit Agrobacterium replication; and
(f) isolating an Agrobacterium cell that has replicated in the culture of step (b),
thereby selecting MDIBOA/DIMBOA a resistant Agrobacterium cell.

22. A phenol hypersensitive Agrobacterium strain comprising selected according to the method:
(d) providing an Agrobacterium cell that comprises an antibiotic resistance gene under the control of a promoter that is upregulated by a vir gene product;
(e) culturing said Agrobacterium cell with varying levels of a phenolic inducer and under other conditions supporting Agrobacterium replication, but including antibiotic resistance to which is provided by the antibiotic resistance gene, at concentrations sufficient to inhibit Agrobacterium replication; and
(f) isolating an Agrobacterium cell receiving the lowest level of phenolic induction that has replicated in the culture of step (b),
thereby selecting a first phenol hypersensitive Agrobacterium cell.

23. A method for producing a MDIBOA/DIMBOA resistant Agrobacterium comprising:
(a) providing an Agrobacterium cell;
(b) culturing said Agrobacterium cell with a phenolic inducer and under other conditions supporting Agrobacterium replication, but including
MDIBOA/DIMBOA at concentrations sufficient to inhibit Agrobacterium replication;
(c) isolating an Agrobacterium cell that has replicated in the culture of step (b);
(d) producing a culture of the isolated Agrobacterium.

24. A method for producing a phenol hypersensitive Agrobacterium strain comprising:
(a) providing an Agrobacterium cell that comprises an antibiotic resistance gene under the control of a promoter that is upregulated by a vir gene product;
(b) culturing said Agrobacterium cell with varying levels of a phenolic inducer and under other conditions supporting Agrobacterium replication, but including
antibiotic resistance to which is provided by the antibiotic resistance gene, at concentrations sufficient to inhibit Agrobacterium replication;
(c) isolating an Agrobacterium cell receiving the lowest level of phenolic induction that has replicated in the culture of step (b); and
(d) producing a culture of the isolated Agrobacterium.

25. A DNA library prepared from an Agrobacterium cell of claim 23.

26. A DNA library prepared from an Agrobacterium cell of claim 24.

27. A method for transducing a plant using a MDIBOA/DIMBOA resistant Agrobacterium strain comprising:
(a) providing a plant cell;
(b) contacting said plant cell with said MDIBOA/DIMBOA resistant Agrobacterium; and
(c) culturing said plant cell under conditions suitable for Agrobacterium-mediated transformation.

28. The method of claim 27, wherein said plant cell is a monocot.

29. The method of claim 27, wherein said plant cell is a dicot.

30. The method of claim 27, wherein said Agrobacterium harbors a heterologous gene.

31. The method of claim 30, wherein said heterologous gene affects one or more performance traits in a plant of said plant cell.

32. The method of claim 27, further comprising obtaining seed of said plant.

33. The method of claim 27, further comprising obtaining progeny of said plant.

34. A method for transducing a plant using a phenol hypersensitive Agrobacterium strain
(a) providing a plant cell;
(b) contacting said plant cell with said phenol hypersensitive Agrobacterium; and
(c) culturing said plant cell under conditions suitable for Agrobacterium-mediated transformation.

35. The method of claim 34, wherein said plant cell is a monocot.

36. The method of claim 34, wherein said plant cell is a dicot.

37. The method of claim 34, wherein said Agrobacterium harbors a heterologous gene.

38. The method of claim 37, wherein said heterologous gene affects one or more performance traits in a plant of said plant cell.

39. The method of claim 34, further comprising obtaining seed of said plant.

40. The method of claim 34, further comprising obtaining progeny of said plant.

41. A plasmid comprising the P_{N25} promoter of coliphage T5 and the virA and virG genes of transcriptional control of said promoter.

42. The plasmid of claim 41, wherein said plasmid backbone is derived from a broad host range vector.

43. The plasmid of claim 42, wherein said broad host range vector is pJB20.

44. The plasmid of claim 41, wherein said virA gene encodes a functional deletion mutant of virA.

45. The plasmid of claim 44, wherein said functional deletion mutant lacks the first 284 amino acid residues of VirA.

46. A host cell comprising the plasmid of claim 41.
